(19) United States
(12) Patent Application Publication
(10) Pub. No.: US 2012/0245107 A1
(43) Pub. Date: Sep. 27, 2012

(54) METHODS OF REDUCING THE PROLIFERATION AND VIABLILITY OF MICROBIAL AGENTS

(75) Inventors: William Henry, Haddenham (GB); Henk-Andre Kroon, Westport, CT (US); Linda Summerton, Harpenden (GB)

(73) Assignee: Targeted Delivery Technologies Limited

(21) Appl. No.: 13/491,474

(22) Filed: Jun. 7, 2012

Related U.S. Application Data
(63) Continuation of application No. 12/508,494, filed on Jul. 23, 2009, now abandoned.
(60) Provisional application No. 61/150,288, filed on Feb. 5, 2009.

(51) Int. Cl. A01N 25/00; A01N 43/653; A61K 31/137; A61K 31/4196; A61K 31/506; A01N 31/04; A01N 43/40; A01N 43/90; A01N 43/60; A01N 33/08; A01N 43/16; A01N 43/22; A01N 43/72; A01N 37/18; A01N 43/36; A01N 37/20; A61K 47/24; A01P 3/00; A61P 31/10; A01P 1/00; A61P 31/04; A61P 31/06; C12Q 1/18; A01N 33/04

(52) U.S. Cl. 514/24; 514/655; 514/383; 514/256; 514/772; 514/730; 514/355; 514/254.11; 514/255.06; 514/669; 514/37; 514/29; 514/152; 514/230.2; 514/628; 435/32

(57) ABSTRACT

The invention relates to formulations of an antimicrobial agent, a lipid, and optionally a surfactant, and uses thereof for reducing the proliferation and viability of microbial agents.

### 1. PRIORITY

This application is a continuation of U.S. application Ser. No. 12/508,494 filed Jul. 23, 2009, which claims the benefit of U.S. Provisional Application No. 61/150,288, filed Feb. 5, 2009, which are incorporated herein by reference in their entireties and for all purposes.

### 2. FIELD OF INVENTION

The invention relates to formulations of an antimicrobial agent, a lipid, and optionally a surfactant, and uses thereof for reducing the proliferation and viability of microbial agents.

### 3. BACKGROUND OF THE INVENTION

The treatment of various diseases in humans, animals and plants is often hampered by the presence of barriers that have low permeability to therapeutic agents. The skin, for example, is fairly impenetrable and as such, many common therapeutic agents must be applied parenterally, i.e., via intravenous, intramuscular, or intradermal administration. Fingernails and toenails also serve as barriers in the treatment of onychomycosis, a fungal infection of the fingernails and toenails that results in thickening, discoloration, splitting of the nails and lifting of the nails from the nail bed. In the case of bacterial infections, gram-negative bacteria, mycobacteria and mycoplasma are unusually successful in surviving in the presence of toxic compounds because they produce effective permeability barriers, comprising the outer membrane and the mycolate-containing cell wall, on the cell surface. In additiona, the transport of different agents into plant tissues is subject to even more severe constraints due to the high permeability barrier of the cuticular wax layers. Thus, noninvasive delivery of therapeutic agents across biological barriers would be advantageous in treating several diseases.

### 4. SUMMARY OF THE DISCLOSURE

Applicant has surprisingly determined that the efficacy of action of an antimicrobial agent can be significantly enhanced by formulation with appropriate lipids and optionally surfactants. In one example, applicant has determined that the action of an antifungal agent can be accelerated (e.g., there is a faster killing time) and that an antifungal agent can even have a different mechanism of action when present in such formulations. Applicant has also determined that such antifungal formulations result in a more even distribution of an antifungal agent throughout a mycotic agent and thus leads to more comprehensive killing of the fungus. Applicant has further determined that such antifungal formulations can lead to a decrease in sporulation of mycotic agents. These findings allow other antimicrobial agents to be formulated with appropriate lipids and optionally surfactants to enhance their activity and thereby allow use of otherwise poorly active agents for new treatment regimes. In an embodiment, the efficacy of action of an antimicrobial agent can be enhanced by formulation in a lipid based particulate.

Provided herein are antimicrobial formulations which may be used to reduce the proliferation or viability of a microbial agent, including fungi, bacteria, and mycoplasma. For example, the formulations are used to inhibit sporulation of a microbial agent. The formulations are also used for screening compounds for antimicrobial activity. The formulations provided herein comprise one or more antimicrobial agents, one or more lipids, and optionally one or more surfactants in a pharmaceutically acceptable carrier.

Provided herein are examples of antimicrobial agents that may be efficaciously formulated to treat a human, an animal, or a plant that has been infected with a microbial agent, including fungi, bacteria, and mycoplasma.

Specific examples of antifungals include, but are not limited to, 5-fluorocytosine, Abafungin, Acrisorcin, Amorolfinc, Albaconazole, Albendazole, Amoroltine, Amphotericin B. Anidulafungin, Arasertaconazole, Azithromycin, Becliconazole, Benzodithiazole, Bifonazole, Butenafine, Butoconazole, Calbistrin, Caspofungin, Chloroxine, Chlorphenesin, Ciclopiroxolamine, Ciclopirox, Cioteronel, Clotrimazole, Croconazole, Cytoporins, Deoxymulundocandin, Eberconazole, Econazole, Efungumab, Fenticonazole, Flavanoid glycosides, Fluconazole, Flutrimazole, Flucytosine, Fosfluconazole, Genaconazole, Gentian violet, Griseofulvin, Griseofulvin-PEG, Haloprogin, Hydroxy itraconazole, Isoconazole, Itraconazole, Ketoconazole, Lanoconazole, Letrazuril, Liranaftate, Luliconazole, Micafungin, Miconazole, Mycophenolic acid, Naftifine, N-chlorotaurine, Natamycin, Nitazoxanide, Nitro-ethylene based antifungals, Nystatin, Omoconazole, Oxiconazole, Polyene macrolide, Posaconazole, Pramiconazole, Quinolone analogs, Rapamycin, Ravuconazole, Rilopirox, Samidazole, Sertaconazole, Sitamaquine, Sordaricin, Squalestatin, Squalene, a Squaline Expoxidase Inhibitor, Sulconazole, Sultriecin, Tafenoquine, Terbinafine, Terconazole, Tioconazole, Tolnaftate, and Voriconazole, or a compound of Formula I:

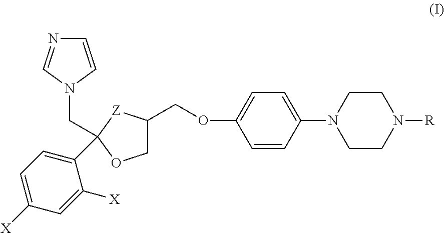

or a single enantiomer, a mixture of enantiomers, or a mixture of diastereomers thereof; or a pharmaceutically acceptable solvate, hydrate, or salt thereof; where R is C₁₋₁₂ alkyl, C₁₋₁₂ acyl, or heteroaryl-C₆₋₁₄ aryl; X is halo; Y is N or CH; and is CH₂ or O, or combinations of any of the above. In certain embodiments, the antifungal formulations provided herein comprise one of itraconazole, ketoconazole, posaconazole, saperconazole, SCH-50002, terconazole, butenafine, and griseofulvin; and hydrates, solvates, and salts thereof; one or more phospholipids, and optionally one or more nonionic surfactants. In an embodiment of the invention, two or more antifungal agents may be formulated together. The disclosure relates to formulations, such as solutions, suspensions, gels, fluid gels, emulsions, emulsion gels, lotions, ointments, film farming solutions, creams, sprays, and lacquers. In one embodiment, the antifungal formulations provided herein comprise an antifungal agent that is from a class of antifungal agents that include, but are not limited to antimetabolites, macrolides, echinocadins, imidazoles, triazoles, benzylamines, echinocadins, griseofulvins, allylamines, polyenes, thiocarbamates, and halogenated phenol ethers.

The antifungal formulations provided herein facilitate the uptake of the antifungal by the phospholipid membranes of the hypha of a mycotic agent. In certain embodiments, the antifungal formulations facilitate the uptake of the antifungal by the Spitzenkorper or Polarisome regions of the hypha of a mycotic agent. Embodiments provided herein are useful in preparations for the application, administration and/or transport of the antifungal, especially for medicinal or biological purposes, into and through barriers and constrictions, such as phospholipid membranes of the Spitzenkorper or Polarisome regions of the hypha of a mycotic agent.

In particular, the disclosure encompasses methods for reducing the proliferation or viability of a mycotic agent comprising contacting said mycotic agent with an effective amount of an antifungal agent, wherein said antifungal agent is formulated with a lipid and a surfactant, and wherein said antifungal agent is adsorbed by phospholipid membranes of the Spitzenkorper or Polarisome regions of the hypha of said mycotic agent. The disclosure also encompasses methods of inhibiting the sporulation of a mycotic agent, comprising contacting said mycotic agent with an effective amount of one or more antifungal agents, wherein said antifungal agent is formulated with a lipid and a surfactant, and wherein said antifungal agent is adsorbed up by phospholipid membranes of the Spitzenkorper or Polarisome regions of the hypha of said mycotic agent. The disclosure further encompasses methods of screening compounds for antifungal activity comprising contacting a mycotic agent with an effective amount of a compound, wherein said compound is formulated with a lipid and a surfactant, and detecting a reduction in the proliferation or viability of said mycotic agent, wherein said compound is adsorbed by the phospholipid membranes of the Spitzenkorper or Polarsiome regions of the hypha of said mycotic agent.

Specific examples of mycotic agents include, but are not limited to, Aspergillus Aspergillus fumigatus, Dermatophytes, Trichophyton rubrum, Trichophyton mentagrophytes, and Epidermophyton floccusum, Candida albicans, Malassezia furfur, Microsporum canis Trichophyton tonsurans, Microsporum audouini, Microsporum gypseum, Trichophyton rubrum, Trichophyton tonsurans, Trichophyton mentagrophytes, Trichophyton interdigitalis, Trichophyton verrucosum, Trichophyton sulphureum, Trichophyton schoenleini, Trichophyton megnini, Trichophyton gallinae, Trichophyton crateriform, Trichomonas and Haemophilus vaginalis, Trypanosoma brucei, and Trypanosoma cruzi. Further examples of mycotic agents can be found in Section 4.1.1.

Also provided herein are antibacterial formulations which may be used to reduce the proliferation or viability of bacterial agents. The formulations can, for example, comprise one or more antibacterial agents, one or more lipids, and optionally one or more surfactants in a pharmaceutically acceptable carrier, wherein the antibacterial is benzyl alcohol, methyl paraben ethanol, isopropanol, glutaraldehyde, formaldehyde, a chlorine compound, and iodine compound, hydrogen peroxide, peracetic acid, ethylene oxide, triclocarban, chlorhexidine, alexidine, triclosan, hexachlorophene, polymeric biguanides, formaldehyde, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof. In one embodiment, the antibacterial agent is an antibiotic. Specific examples of antibiotics include, but are not limited to aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof.

The antibacterial formulations provided herein facilitate the uptake of the antibacterial by the phospholipid membranes of a bacterium. In an embodiment, the antibacterial formulations are used to inhibit sporulation of a bacterium. Embodiments provided herein are useful in preparations for the application, administration and/or transport of the antibacterial, especially for medicinal or biological purposes, into and through barriers and constrictions, such as phospholipid membranes of a bacterium.

In particular, the disclosure encompasses methods for reducing the proliferation or viability of a bacterium, comprising contacting said bacterium with an effective amount of one or more antibacterial agents, wherein said antibacterial agent is formulated with a lipid and optionally a surfactant, and wherein said antibacterial agent is adsorbed by the phospholipid membranes of the bacterium. The disclosure also encompasses methods of inhibiting the sporulation of a bacterium, comprising contacting said bacterium with an effective amount of an antibacterial agent, wherein said antibacterial agent is formulated with a lipid and a surfactant, and wherein said antibacterial agent is adsorbed by the phospholipid membranes of the bacterium. Specific examples of bacteria include, but are not limited to E. coli, Klebsiella, Staphylococcus, Streptococcus, Haemophilus influenzae, Neisseria gonorrhoeae, Pseudomonas, Clostridium, Enterococcus, Bacillus, Acinetobacter baumannii, M. tuberculosis, Chlamydia, N. gonorrhea, Shigella, Salmonella, Proteus, Garcinerella, Nocardia, Nocardia asteroides, Planococcus, Corynebacteria, Rhodococcus, Vibrio, Cholera, Treponema pallidua, Pseudomonas, Bordetella pertussis, Brucella, Franciscella tulorensis, Helicobacter pylori, Leptospria interrogates, Legionella pneumophila, Yersinia, Pneumococcus, Meningococcus, Hemophilus influenza, Toxoplasma gondic, Complylobacteriosis, Moraxella catarrhalis, Donovanosis, and Actinomycosis. Further examples of bacteria can be found herein, in Section 4.1.2.

In one embodiment, the bacterium is a mycobacterium. In a specific embodiment, the mycobacterium is Mycobacterium tuberculosis. Examples of antibacterials that can be used to inhibit the proliferation or viability of Mycobacterium tuberculosis include, but are not limited to Isoniazid, Rifampin, Pyrazinamide, Ethambutol, and Streptomycin.

In another embodiment, the bacterium is a mycoplasma. Examples of mycoplasma include, but are not limited to, Mycoplasma (M.) buccale, M. fancium, M. fermenians, M. Genitalium, M. hominis, M. lipophilum, M. oral, M. penetrans, M. pneumoniae, M. salivaritum, or M. spermatophilum. Examples of agents, in particular antibiotics, that can be used to inhibit the proliferation or viability of a mycoplasma include, but are not limited to, erythromycin, azithromycin, clarithromycin, tetracycline, doxycycline, minocycline, clindamycin, ofloxacin, and chloramphenicol.

Many assays well-known in the art can be used to assess the proliferation and viability of microbial agents following exposure to the formulations provided herein. For example, proliferation of microbial agents can be assayed by measuring Bromodeoxyuridine (BrdU) incorporation, (3H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription.

Cell viability can be assessed by using trypan-blue staining or other cell death or viability markers known in the art. In a specific embodiment, the level of cellular ATP is measured to determined cell viability. In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, formation of vacuoles, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic-40%), Ps (partially toxic-slight-20%), or 0 (no toxicity-0%), conforming to the degree of cytotoxicity seen. A 50% cell inhibitory (cytotoxic) concentration (IC₅₀) is determined by regression analysis of these data.

Any assay well known in the art can be used to determine the spore count of microbial agents following exposure to the formulations provided herein. For example, the viable microbial spore count can be measured by colony counting, then the total microbial spore count can be measured by direct microscopic counting, the procedures for which are described in more detail in Section 4.9. The ratio of viable to total microbial spore count yields the fraction of spores that remain viable within a given sample.

In one embodiment, the formulations provided herein are administered to a human in order to reduce the proliferation or viability of a microbial agent that has infected said human. In another embodiment, the formulations provided herein are administered to an animal in order to reduce the proliferation or viability of a microbial agent that has infected said animal. In yet another embodiment, the formulations provided herein are delivered to a plant in order to reduce the proliferation or viability of a microbial agent that has infected said plant.

In one embodiment, the formulations provided herein are administered to a human in order to reduce the sporulation of a microbial agent that has infected said human. In another embodiment, the formulations provided herein are administered to an animal in order to reduce the sporulation of a microbial agent that has infected said animal. In yet another embodiment, the formulations provided herein is delivered to a plant in order to reduce the sporulation of a microbial agent that has infected said plant.

The formulations may be administered to a human or animal topically, including mucosal delivery. Mucosal delivery includes pulmonary, oropharyngeal, genitourinary, ocular, and nasal delivery. Pulmonary administration can be employed, e.g., by use of an inhaler or nebulizer, and formulation with an aerosolizing agent, or via perfusion in a fluorocarbon or synthetic pulmonary surfactant. In certain embodiments, the formulations provided herein can be formulated as a suppository, with traditional binders and carriers such as triglycerides.

In an embodiment, the formulations provided therein are lyophilized to allow for pulmonary delivery. The formulations provided herein can be lyophilized by mixing the formulation with a diluent to form a liquid composition and then lyophilizing the liquid composition to form a lyophilate. The formulations may be lyophilized by any method known in the art for lyophilizing a liquid.

In one embodiment, the formulations provided herein are to be administered or delivered for a period of ten to twelve weeks. In another embodiment, the formulations are administered or delivered for a prolonged period of time, up to forty eight weeks. The formulation is to be administered or delivered for a period of time to result in a microbial cure rate, preferably greater than about 70%, 75%, 80%, 85%, 90%, 95%, 96%, 97%, 98%, or 99% in a subject.

Examples of lipid based formulations that can be used in the methods described herein include, but are not limited to emulsions, nanoemulsions, vesicles, liposomes, micelles, microspheres, nanospheres, emulsions, lipid discs, and non-specific lipid conglomerates.

The formulations provided herein may have a range of lipid to surfactant ratios. The ratios may be expressed in terms of molar terms (mol lipid/mol surfactant). The molar ratio of lipid to surfactant in the formulations provided herein may be from about 1:2 to about 10:1. In certain embodiments, the ratio is from about 1:1 to about 2:1, from about 2:1 to about 3:1, from about 3:1 to about 4:1, from about 4:1 to about 5:1, or from about 5:1 to about 10:1. In specific embodiments, the lipid to surfactant ratio is about 1.0, about 1.25, about 1.5, about 1.75, about 2.0, about 2.5, about 3.0, or about 4.0.

The formulations provided herein may have varying ratios of the antimicrobial to lipid. The ratios may be expressed in terms of molar ratios (mol antimicrobial/mol lipid). The molar ratio of the antimicrobial to lipid in the formulations provided herein may be from about 1:50 to about 50:1, from about 1:25 to about 25:1, from about 1:10 to about 10:1, from about 1:5 to about 5:1, from about 1:50 to about 50:1, or from about 0.2:1 to about 2:1. In certain embodiments, the ratio is from about 0.2:1 to about 0.7:1, from about 0.7:1 to about 1.2:1, from about 1.2:1 to about 1.7:1, or from about 1.7:1 to about 2:1.

In some embodiments, the lipid in the formulations provided herein is a phospholipid. In one embodiment, the ratio of phospholipid to surfactant is 1/1 to 5/1 w/w. In another embodiment, the formulation contains 2.0-10.0% by weight phospholipid. In a more specific embodiment, the formulation contains 1.0-5.0% by weight surfactant. In a particular embodiment, the phospholipid is phosphatidylcholine.

In one embodiment, the surfactant is a nonionic surfactant selected from the group consisting of: polyoxyethylene sorbitans, polyhydroxyethylene stearates or polyhydroxyethylene laurylethers. In a more specific embodiment, the surfactant is polysorbate 80 (Tween 80).

In some embodiments, the formulations provided herein comprise from about 1 to about 20 mg of the antimicrobial. For instance, the formulations can comprise about 2, about 3, about 4, about 5, about 6, about 7, about 8, about 9, about 10, about 11, about 12, about 13, about 14, about 15, about 16, about 17, about 18, about 19, or about 20 mg of the antimicrobial.

In some embodiments, the formulations provided herein comprise from about 1 to about 500 μg of the antimicrobial. For instance, the formulations can comprise about 1, about 25, about 50, about 75, about 100, about 125, about 150, about 175, about 200, about 225, about 250, about 275, about 300, about 325, about 350, about 375, about 400, about 425, about 450, about 475, or about 500 μg of the antimicrobial.

In certain embodiments, the formulations provided herein form vesicles or other extended surface aggregates (ESAs), wherein the vesicular preparations have improved permeation capability through the semi-permeable barriers. While not to be limited to any mechanism of action, the formulations provided herein are able to form vesicles characterized by their deformability and/or adaptability. The vesicles' deformability and/or adaptability allow the vesicles to penetrate the pores of the skin and/or nails and deliver the antimicrobial to the site of infection in an amount sufficient to treat the infection. The vesicles' deformability and/or adaptability also allow an antifungal to be adsorbed the phospholipid membranes of the Spitzenkorper or Polarisome regions of the hypha of a mycotic agent. The vesicles' deformability and/or adaptability also allow an antibacterial to be adsorbed by the phospholipid membranes of a bacterium. The adaptability or deformability of the vesicles may be determined by the ability of the vesicles to penetrate a barrier with pores having an average pore diameter at least 50% smaller than the average vesicle diameter before the penetration.

The disclosure further encompasses a method for treating inhalation anthrax in a human subject that has been exposed to Bacillus anthracis spores, said method comprising administering to said human subject a composition comprising an antibacterial agent that is formulated with a lipid and a surfactant, and wherein said antibacterial agent is adsorbed by the phospholipid membrane of said Bacillus anthracis.

The disclosure also encompasses a method for method of preventing the development of inhalation anthrax in a human subject that has been exposed to Bacillus anthracis spores, said method comprising administering to said human subject a composition comprising an antibacterial agent that is formulated with a lipid and a surfactant, and wherein said antibacterial agent is adsorbed the phospholipid membrane of said Bacillus anthracis.

The disclosure also encompasses a method of treating tuberculosis in a human subject that has been infected with Mycobacterium tuberculosis, said method comprising administering to said human subject a composition comprising an antibacterial agent that is formulated with a lipid and a surfactant, and wherein said antibacterial agent is adsorbed by the phospholipid membrane of said Mycobacterium tuberculosis.

The disclosure also encompasses a method of treating pneumonia in a human subject that has been infected with Mycoplasma pneumoniae, said method comprising administering to said human subject a composition comprising an antibacterial agent that is formulated with a lipid and a surfactant, and wherein said antibacterial agent is adsorbed up by the phospholipid membrane of said Mycoplasma pneumoniae.

The disclosure also encompasses a method of reducing the proliferation or viability of a mycotic agent comprising contacting said mycotic agent with an effective amount of one or more antifungal agents, wherein said antifungal agent is formulated with a phospholipid and a surfactant. The disclosure also encompasses a method of reducing the proliferation or viability of a mycotic agent comprising contacting said mycotic agent with an effective amount of a combination of antifungal agents, wherein one or more of the antifungal agents is/are formulated with a phospholipid and a surfactant. The effect of contacting a mycotic agent with a combination of one or more antifungal agents, wherein one or more of the antifungal agents is/are formulated with a phospholipid and a surfactant may result in a synergistic effect, i.e., the combined effect of one or more antifungal agents on reducing the proliferation or viability of a mycotic agent may be greater than the effect of a single antifungal agent on reducing the proliferation or viability of a mycotic agent. In one particular embodiment, the method of reducing the proliferation or viability of a mycotic agent comprises contacting said mycotic agent with an effective amount of a combination of terbinafine and voriconazole with either antifungal or both being formulated with a phospholipid and surfactant. In another embodiment, the method of reducing the proliferation or viability of a mycotic agent comprises contacting said mycotic agent with an effective amount of a combination of terbinafine formulation and voriconazole formulated with a phospholipid and a surfactant. In a specific embodiment, the method of reducing the proliferation or viability of a mycotic agent comprises contacting an Aspergillus, such as A. fumigatus or A. flavus, with an effective amount of a combination of terbinafine formulation and voriconazole formulated in Transfersome®.

In certain embodiments of the methods, the methods comprise administering to a subject the topical antifungal formulations as described herein in combination with a second antifungal formulation (either topically administered or otherwise). In certain embodiments, the methods comprise contacting a mycotic agent with a combination of more than one antifungal, each independently formulated, e.g., in a Transfersome® or otherwise.

### 5. DETAILED DESCRIPTION OF THE DISCLOSURE

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “hypha” refers to a long, branching filamentous cell of a fungus.

The term “Polarisome” refers to a protein complex found at the tip of a growing fungal hypha and that has a role in determining cell polarity of a fungus.

The term “Spitzenkorper” refers to is an intracellular organelle associated with tip growth of a fungal hypha. It is composed of an aggregation of membrane-bound vesicles that is part of the endomembrane of fungi.

The term “subject” refers to an animal, including, but not limited to, a primate (e.g., human), cow, sheep, goat, pig, horse, dog, cat, rabbit, rat, or mouse. The terms “subject” and “patient” are used interchangeably herein in reference, for example, to a mammalian subject, such as a human subject.

The term “treat,” “treating,” or “treatment of” means that the severity of a subject's condition is reduced or at least partially improved or ameliorated and/or that some alleviation, mitigation or decrease in at least one clinical symptom is achieved and/or there is an inhibition or delay in the progression of the condition and/or delay in the progression of the onset of disease or illness. The term “treat,” “treating,” or “treatment of” also means managing the disease state, e.g., onychomycosis.

The term “pharmaceutically acceptable” when used in reference to the formulations provided herein denotes that a formulation does not result in an unacceptable level of irritation in the subject to whom the formulation is administered. Preferably such level will be sufficiently low to provide a formulation suitable for approval by regulatory authorities.

The term “sufficient amount,” “amount effective to,” or an “amount sufficient to” achieve a particular result refers to an amount of an antimicrobial or a salt thereof that is effective to produce a desired effect, which is optionally a therapeutic effect (i.e., by administration of a therapeutically effective amount). Alternatively stated, a “therapeutically effective” amount is an amount that provides some alleviation, mitigation, and/or decrease in at least one clinical symptom. Clinical symptoms associated with the disorder that can be treated by the methods provided herein are well-known to those skilled in the art. Further, those skilled in the art will appreciate that the therapeutic effects need not be complete or curative, as long as some benefit is provided to the subject. For example, a “sufficient amount” or “an amount sufficient to” can be an amount that is effective to treat onychomycosis, may be defined as a mycological cure.

As used herein with respect to numerical values, the term “about” means a range surrounding a particular numeral value which includes that which would be expected to result from normal experimental error in making a measurement. For example, in certain embodiments, the term “about” when used in connection with a particular numerical value means±1%, ±2%, ±3%, ±4%, ±5%, ±10%, ±15%, or ±20% of the numerical value.

The term “alkyl” refers to a linear or branched saturated monovalent hydrocarbon radical, wherein the alkyl may optionally be substituted with one or more substituents Q as described herein. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 12 (C₁₋₁₂), 1 to 10 (C₁₋₁₀), or 1 to 6 (C₁₋₆) carbon atoms, or a branched saturated monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may also be optionally substituted with one or more substituents Q as described herein.

The term “heteroaryl” refers to a monocyclic aromatic group and/or multicyclic aromatic group that contain at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. The heteroaryl may be attached to the main structure at any heteroatom or carbon atom which results in the creation of a stable compound. In certain embodiments, the heteroaryl has from 5 to 20, from 5 to 15, or from 5 to 10 ring atoms. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. In certain embodiments, heteroaryl may also be optionally substituted with one or more substituents Q as described herein.

The term “alkenoyl” as used herein refers to —C(O)-alkenyl. The term “alkenyl” refers to a linear or branched monovalent hydrocarbon radical, which contains one or more, in one embodiment, one to five, carbon-carbon double bonds. The alkenyl may be optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 30 (C₂₋₃₀), 2 to 24 (C₂₋₂₄), 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 30 (C₃₋₃₀), 3 to 24 (C₃₋₂₄), 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl. In certain embodiments, the alkenoyl is mono-alkenoyl, which contains one carbon-carbon double bond. In certain embodiments, the alkenoyl is di-alkenoyl, which contains two carbon-carbon double bonds. In certain embodiments, the alkenoyl is poly-alkenoyl, which contains more than two carbon-carbon double bonds.

The term “heterocyclyl” or “heterocyclic” refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, acridinyl, azepinyl, benzimidazolyl, benzindolyl, benzoisoxazolyl, benzisoxazinyl, benzodioxanyl, benzodioxolyl, benzofuranonyl, benzofuranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4-dithianyl, furanonyl, furanyl, imidazolidinyl, imidazolyl, imidazopyridinyl, imidazothiazolyl, indazolyl, indolinyl, indolizinyl, indolyl, isobenzotetrahydrofuranyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthyridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4-piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydrofuranyl, tetrahydroisoquinolinyl, tetrahydropyranyl, tetrahydrothienyl, tetrazolyl, thiadiazolopyrimidinyl, thiadiazolyl, thiamorpholinyl, thiazolyl, thienyl, triazinyl, triazolyl, and 1,3,5-trithianyl. In certain embodiments, heterocyclic may also be optionally substituted with one or more substituents Q as described herein.

The term “halogen”, “halide” or “halo” refers to fluorine, chlorine, bromine, and/or iodine.

The term “optionally substituted” is intended to mean that a group, including alkyl, alkenyl, alkynyl, cycloalkyl, aryl, aralkyl, heteroaryl, and heterocyclyl, may be substituted with one or more substituents Q, in one embodiment, one, two, three or four substituents Q, where each Q is independently selected from the group consisting of cyano, halo, oxo, nitro, C₁₋₆ alkyl, halo-C₁₋₆ alkyl, C₂₋₆ alkenyl, C₂₋₆ alkynyl, C₃₋₇ cycloalkyl, C₆₋₁₄ aryl, C₇₋₁₄ aralkyl, heteroaryl, heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, R^{g}, and R^{h} is independently hydrogen, C₁₋₆ alkyl, C₂₋₆, alkenyl, C₂₋₆ alkynyl, C₃₋₇ cycloalkyl, C₆₋₁₄ aryl, C₇₋₁₄ aralkyl, heteroaryl, or heterocyclyl; or R^{f} and R^{g} together with the N atom to which they are attached form heterocyclyl.

The terms “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “solvate” refers to a compound provided herein or a salt thereof, which further includes a stoichiometric or non-stoichiometric amount of solvent bound by non-covalent intermolecular forces. Where the solvent is water, the solvate is a hydrate.

The formulations provided herein comprise an antifungal or an antibacterial, a lipid, preferably a phospholipid, a surfactant, preferably a nonionic surfactant, and an aqueous solution, having a pH ranging from 3.5 to 9.0, preferably from 4 to 7.5. The antifungal formulations provided herein may contain an antifungal, or a pharmaceutically acceptable solvate, hydrate, or salt of the antimicrobial. The formulations may optionally contain buffers, antioxidants, preservatives, microbicides, antimicrobials, and/or thickeners. In certain embodiments, a certain portion of the antimicrobial in the pharmaceutical composition is in salt form.

While not to be limited by any mechanism of action, the formulations provided herein form vesicles or other extended surface aggregates (ESAs), wherein the vesicular preparations have improved permeation capability through the semi-permeable barriers, such as skin and/or nails. The vesicles or extended surface aggregates provided herein comprise of an antifungal or an antibacterial, a lipid, and one or more membrane destabilizing agents, such as surfactants.

4.1. Microbial Agents

4.1.1. Mycotic Agents

Specific examples of mycotic agents that can infect humans and animals include, but are not limited to, Trichophyton rubrum, Trichophyton mentagrophytes, and Epidermophyton floccusum, Candida (e.g., Candida (C.) albicans, C. glabrata C. krusei, C. tropicalis), Cryptococcus (e.g. Cryptococcus neoformans), Dermatophytes, Malassezia furfur, Microsporum canis, Trichophyton tonsurans, Microsporum audouini, Microsporum gypseum, Trichophyton rubrum, Trichophyton tonsurans, Trichophyton mentagrophytes, Trichophyton interdigitalis, Trichophyton verrucosum, Trichophyton sulphureum, Trichophyton schoenleini, Trichophyton megnini, Trichophyton gallinae, Trichophyton crateriform, Trichomonas and Haemophilus vaginalis, Blastomyces dermatitis, Coccidioides immitis, Histoplasma capsulatum, and Sporothrix schenckii, Trypanosoma (e.g., Trypanosoma (T.) ambystoma, T. avium, T. boissoni, T. brucei, T. carassii, T. cruzi, T. congolense, T. equinum, T. equiperdum, T. evansi, T. everetti, T. hosei, T. levisi, T. melophagium, T. paddai, T. parroti, T. percae, T. rangeli, T. rotatorium, T. rugosae, T. sergenti, T. simiae, T. sinipercae, T. suis, T. theileri, T. teleosts, T. nagana), Aspergillus fumigatus, Aspergillus flavus, and Aspergillus clavatus.

Specific examples of mycotic agents that can infect plants include, but are not limited to, Basidiomycetes (e.g., Puccinia spp., Cronartium ribicola, and Gymnosporangium juniperi-virginianae), the smut fungi, (e.g., Ustilago spp.), Gaeumannomyces graminis var tritici, Physoderma alfalfae, Glomerella cingulata, Gymnosporangium juniperi-virginianae, Venturia inaequalis, Fusarium oxysporum f. cubense, Ustilago nuda Rostr., Septoria apiicola, Fusarium oxysporum f. apii Claviceps purpurea, Puccinia spp., P. graminis, Phytopthera infestans, and Armillaria mellae.

4.1.2. Bacterial Agents

Specific examples of bacterial agents that can infect humans and animals include, but are not limited to, E. coli, Klebsiella (e.g., Klebsiella pneumoniae and Klebsiella oxytoca). Staphylococcus (e.g. Staphylococcus aureus), Streptococcus (e.g., Streptococcus pneumoniae), Haemophilus influenzae, Neisseria gonorrhoeae, Pseudomonas Pseudomonas aeruginosa), Clostridium (e.g., Clostridium (C.) tetani, C. botulinum, C. perfringens), Enterococcus, Bacillus (e.g. Bacillus (B.) anthracis, B. cereus, B. circulans, B. subtilis, B. megaterium), Acinetobacter baumannii, M. tuberculosis, Chlamydia, N. gonorrhea, Shigella, Salmonella, Proteus, Gardnerella, Nocardia, Nocardia asteroides, Planococcus, Corynebacteria, Rhodococcus, Vibrio (e.g., Vibrio Cholera, Treponema pallidua, Pseudomonas, Bordetella pertussis, Brucella, Franciscella tulorensis, Helicobacter pylori, Leptospria interrogaus, Legionella pneumophila, Yersinia (e.g. Yersinia (Y.) pestis Y enterocolitical, pseudotuberculosis, Streptococcus (types A and B), Pneumococcus, Meningococcus, Hemophilia influenza (type b), Toxoplasma gondic, Complylobacteriosis, Moraxella catarrhalis, Donovanosis, and Actinomycosis.

In one embodiment, the bacterium is a mycobacterium. In a specific embodiment, the mycobacterium is Mycobacterium tuberculosis.

In another embodiment, the bacterium is a mycoplasma. Examples of mycoplasma include, but are not limited to, Mycoplasma (M.) buccale, M. faucium, M. fermentans, M. Genitalium, M. hominis, M. lipophilum, M. oral, M. penetrans, M. pneumoniae, M. salivarium, or M. spermatophilum.

In an embodiment, the bacterium is a methicillin-resistant stapholococcus aureus (MRSA). In an embodiment, the bacteria used in the methods of the invention are antibiotic resistant.

Specific examples of bacteria that can infect plants include, but are not limited to, Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasm.

4.2. Antifungals

4.2.1. Allyamines

Allyamines that are suitable for use in the topical antifungal formulations provided herein include, but are limited to, amorolfine, butenafine, and naftifine.

In one embodiment, the allyamine in the topical antifungal formulations provided herein is amorolfine having the structure of:

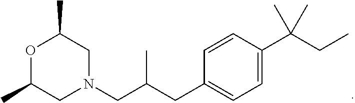

In another embodiment, the allyamine in the topical antifungal formulations provided herein is butenafine having the structure of:

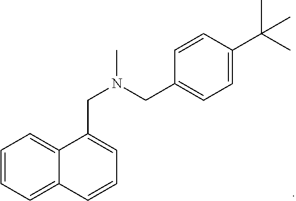

In yet another embodiment, the allyamine in the topical antifungal formulations provided herein is naftifine having the structure of:

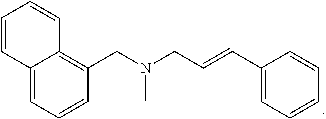

The allyamine may be used in the formulations provided herein in its free base, or its pharmaceutically acceptable solvate, hydrate, or salt form. In a specific embodiment, the allyamine is used as a hydrochloride (HCl) salt. The term “allyamine” as used herein includes the free base form of the compound as well as pharmaceutically acceptable solvate, hydrate, or salt form. Suitable salt forms include, but not are limited to chloride, bromide, iodide, acetate, and fumarate.

The pharmaceutical formulations provided herein allow for the topical administration of the allyamine, and comprise a therapeutically effective amount of the allyamine and at least one lipid and at least one surfactant, wherein the formulation comprises 0.25-25.0% of the allyamine in terms of dry “total lipid” weight being defined as the sum total of dry weights of all included lipids, surfactants, lipophilic excipients, and the allyamine. The formulations provided herein may also comprise 0.25 to 30% by weight of the allyamine. In specific embodiments, the topical formulations may comprise from about 0.25% to about 0.5%, from about 0.5% to about 1%, from about 1% to about 1.5%, from about 1.5% to about 2%, from about 2% to about 2.5%, from about 2.5% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 12%, from about 12%, to about 14%, from about 14% to about 16%, from about 16% to about 18%, from about 18% to about 20%, from about 22% to about 24%, from about 26% to about 28%, or from about 28% to about 30% by weight of the allyamine.

The pharmaceutical formulations provided herein contain the allyamine in an amount ranging from about 0.25 mg/g to about 200 mg/g. In certain embodiments, the amount of the allyamine in the pharmaceutical formulations may range from about 0.25 mg/g to about 200 mg/g, from about 0.5 mg/g to about 175 mg/g, from about 0.5 mg/g to about 150 mg/g, from about 0.5 mg/g to about 100 mg/g, from about 0.5 mg/g to about 75 mg/g, from about 0.5 mg/g to about 50 mg/g, from about 0.5 mg/g to about 25 mg/g, from about 0.5 mg/g to about 20 mg/g, from about 0.5 mg/g to about 10 mg/g, from about 0.5 mg/g to about 5 mg/g, from about 0.5 mg/g to about 4 mg/g, from about 0.5 mg/g to about 3 mg/g, from about 0.5 mg/g to about 2 mg/g, or from about 0.5 mg/g to about 1.5 mg/g.

In certain embodiments, the topical formulations provided herein also comprise a polar liquid medium. In certain embodiments, the topical formulations provided herein are administered in an aqueous medium. The topical formulations provided herein may be in the form of a solution, suspension, gel, fluid gel, emulsion, emulsion gel, cream, lotion, ointment, spray, film forming solution, lacquer or a patch soaked with the formulation.

4.2.2. Triazoles and Imidazoles

Triazole and imidazole antifungals that are suitable for use in the topical antifungal formulations provided herein have the structure of Formula I:

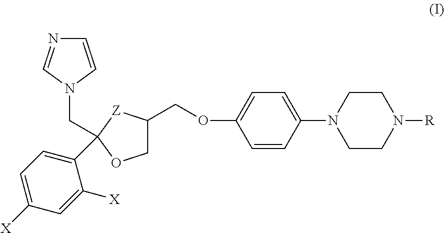

or a single enantiomer, a mixture of enantiomers, or a mixture of diastereomers thereof; or a pharmaceutically acceptable solvate, hydrate, or salt thereof; wherein:

R is C₁₋₁₂ alkyl, C₁₋₁₂ acyl, or heteroaryl-C₆₋₁₄ aryl;

X is halo;

Y is N or CH; and

Z is CH₂ or O.

The groups, R, X, Y, and Z in Formula I are further defined herein. All combinations of the embodiments provided herein for such groups are within the scope of this disclosure.

In certain embodiments, R is C₁₋₁₂ alkyl. In certain embodiments, R is isopropyl. In certain embodiments, R is C₁₋₁₂ acyl. In certain embodiments, R is acetyl. In certain embodiments, R is heteroaryl-C₆₋₁₄ aryl. In certain embodiments, R is 1-see-butyl-1H-1,2,4-triazol-5(4H)-one-4-yl, 1-(2-hydroxypentan-3-yl)-1H-1,2,4-triazol-5(4H)-one-4-yl, or 1-((2S,3R)-2-hydroxypentan-3-yl)-1H-1,2,4-triazol-5(4H)-one-4-yl.

In certain embodiments, each X is independently fluoro or chloro. In certain embodiments, X is fluoro. In certain embodiments, X is chloro.

In certain embodiments, Y is N. In certain embodiments, Y is CH.

In certain embodiments, Z is CH₂. In certain embodiments, Z is O.

In one embodiment, provided herein is a compound of Formula I, wherein R is isopropyl, acetyl, 1-sec-butyl-1H-1,2,4-triazol-5(4H)-one-4-yl, 1-(2-hydroxypentan-3-yl)-1H-1,2,4-triazol-5(4H)-one-4-yl, or 1-((2S,3R)-2-hydroxypentan-3-yl)-1H-1,2,4-triazol-5(4H)-one-4-yl; each X is independently fluoro or chloro; Y is N or CH; and Z is CH₂ or O.

In one embodiment, the compound of Formula I is itraconazole having the structure of:

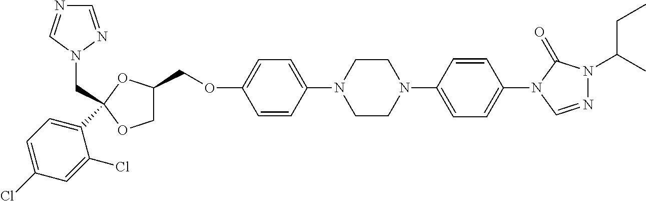

or a single enantiomer or a mixture of diastereomers thereof; or a pharmaceutically acceptable solvate, hydrate, or salt thereof.

In another embodiment, the compound of Formula I is ketoconazole having the structure:

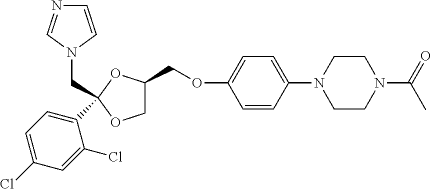

or a pharmaceutically acceptable solvate, hydrate, or salt thereof.

In yet another embodiment, the compound of Formula I is posaconazole having the structure of:

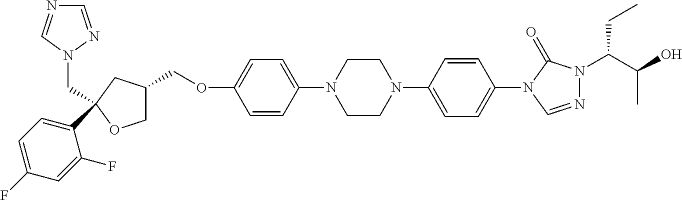

or a pharmaceutically acceptable solvate, hydrate, or salt thereof.

In yet another embodiment, the compound of Formula I is terconazole having the structure of:

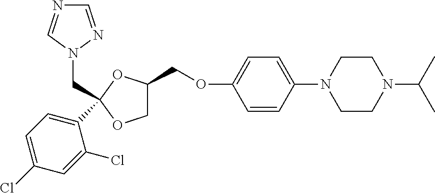

or a pharmaceutically acceptable solvate, hydrate, or salt thereof.

In yet another embodiment, the compound of Formula I is SCH-50002 having the structure of:

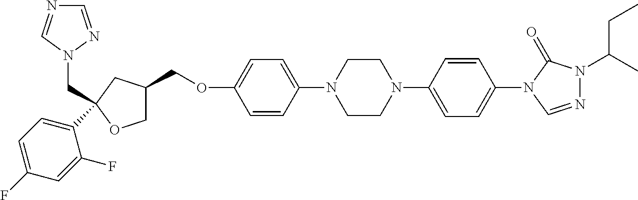

or a single enantiomer or a mixture of diastereomers thereof; or a pharmaceutically acceptable solvate, hydrate, or salt thereof.

In still another embodiment, the compound of Formula I is saperconazole having the structure of:

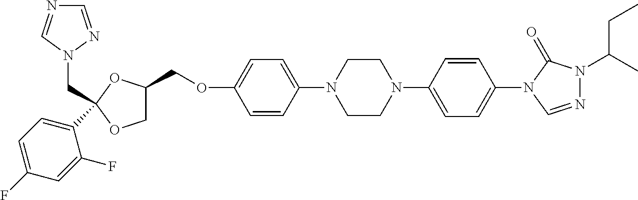

or a single enantiomer or a mixture of diastereomers thereof; or a pharmaceutically acceptable solvate, hydrate, or salt thereof.

Triazole and imidazole antifungals as provided herein may be used in the formulations provided herein as a single enantiomer, a mixture of enantiomers, or a mixture of diastereomers thereof; or a pharmaceutically acceptable solvate, hydrate, or salt thereof. In a specific embodiment, triazole and imidazole antifungals are used in their free base forms. The term “a triazole and imidazole antifungal” as used herein includes the free base form of the compound, including single enantiomers, mixtures of enantiomers, and mixtures of diastereomers of the compound; as well as pharmaceutically acceptable solvates, hydrates, and salts of the compound, including its single enantiomers, mixtures of enantiomers, and mixtures of diastereomers.

The pharmaceutical formulations provided herein allow for the topical administration of triazole and imidazole antifungals, particularly, itraconazole, ketoconazole, posaconazole, saperconazole, SCH-50002, and terconazole, and comprise a therapeutically effective amount of a triazole or imidazole antifungal provided herein, and at least one lipid and at least one surfactant, wherein the formulation comprises 0.25-25% of the antifungal in terms of dry “total lipid” weight being defined as the sum total of dry weights of all included lipids, surfactants, lipophilic excipients, and the antifungal. The formulations provided herein may also comprise 0.25 to 30% by weight of the antifungal. In specific embodiments, the topical anti fungal formulations may comprise from about 0.25% to about 0.5%, from about 0.5% to about 1%, from about 1% to about 1.5%, from about 1.5% to about 2%, from about 2% to about 2.5%, from about 2.5% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 12%, from about 12% to about 14%, from about 14% to about 16%, from about 16% to about 18%, from about 18% to about 20%, from about 22% to about 24%, from about 26% to about 28%, or from about 28% to about 30% by weight of the triazole or imidazole antifungal.

The pharmaceutical formulations provided herein contain the triazole or imidazole antifungal in an amount ranging from about 0.25 mg/g to about 200 mg/g. In certain embodiments, the amount of the triazole or imidazole antifungal in the pharmaceutical formulations may range from about 0.25 mg/g to about 200 mg/g, from about 0.5 mg/g to about 175 mg/g, from about 0.5 mg/g to about 150 mg/g, from about 0.5 mg/g to about 100 mg/g, from about 0.5 mg/g to about 75 mg/g, from about 0.5 mg/g to about 50 mg/g, from about 0.5 mg/g to about 25 mg/g, from about 0.5 mg/g to about 20 mg/g, from about 0.5 mg/g to about 10 mg/g, from about 0.5 mg/g to about 5 mg/g, from about 0.5 mg/g to about 4 mg/g, from about 0.5 mg/g to about 3 mg/g, from about 0.5 mg/g to about 2 mg/g, or from about 0.5 mg/g to about 1.5 mg/g.

In certain embodiments, the antifungal formulations provided herein also comprise a polar liquid medium. In certain embodiments, the antifungal formulations provided herein are administered in an aqueous medium. The antifungal formulations provided herein may be in the form of a solution, suspension, gel, fluid gel, emulsion, emulsion gel, cream, lotion, ointment, spray, film forming solution, lacquer or a patch soaked with the formulation.

The antifungals provided herein are intended to encompass all possible stereoisomers, including enantiomers and diastereomers and mixtures thereof, unless a particular stereochemistry is specified. Where an antifungals provided herein contains an alkenyl or alkenylene group, the antifungal may exist as a cis (Z) or trans (E) isomer or as a mixture of geometric cis/trans (or Z/E) isomers. Where structural isomers are interconvertible via a low energy barrier, the antifungal may exist as a single tautomer or a mixture of tautomers. This can take the form of proton tautomerism in the antifungal that contains, for example, an imino, keto, or oxime group; or so-called valence tautomerism in the antifungal that contain an aromatic moiety. It is understood that a single antifungal may exhibit more than one type of isomerism.

The antifungals provided herein may be enantiomerically pure, such as a single enantiomer or a single diastereomer, or may be stereoisomeric mixtures, such as a mixture of enantiomers, a racemic mixture, or a diastereomeric mixture. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the antifungals provided herein contain an acidic or basic moiety, they may also be provided as pharmaceutically acceptable salts (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 111-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

4.2.3. Liranaftate and Tolnaftate

Liranaftate is an antifungal having the structure of:

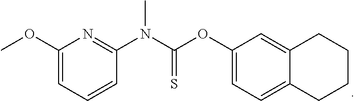

Tolnaftate is an antifungal having the structure of:

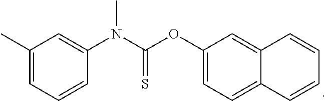

Liranaftate or tolnaftate may be used in the formulations provided herein in its free form, or its pharmaceutically acceptable solvate, hydrate, or salt form. In a specific embodiment, liranaftate or tolnaftate is used in its free form. The term “liranaftate” as used herein includes the free form of the compound as well as pharmaceutically acceptable solvate, hydrate, or salt form. The term “tolnaftate” as used herein includes the free form of the compound as well as pharmaceutically acceptable solvate, hydrate, or salt form.

The pharmaceutical formulations provided herein allow for the topical administration of liranaftate or tolnaftate, and comprise a therapeutically effective amount of liranaftate or tolnaftate and at least one lipid and at least one surfactant, wherein the formulation comprises 0.25-25% liranaftate or tolnaftate in terms of dry “total lipid” weight being defined as the sum total of dry weights of all included lipids, surfactants, lipophilic excipients, and liranaftate or tolnaftate. The formulations provided herein may also comprise 0.25 to 30% by weight of liranaftate or tolnaftate. In specific embodiments, the topical formulations may comprise from about 0.25% to about 0.5%, from about 0.5% to about 1%, from about 1% to about 1.5%, from about 1.5% to about 2%, from about 2% to about 2.5%, from about 2.5% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 12%, from about 12% to about 14%, from about 14% to about 16%, from about 16% to about 18%, from about 18% to about 20%, from about 22% to about 24%, from about 26% to about 28%, or from about 28% to about 30% by weight of liranaftate or tolnaftate.

The pharmaceutical formulations provided herein contain liranaftate or tolnaftate in an amount ranging from about 0.25 mg/g to about 200 mg/g. In certain embodiments, the amount of liranaftate or tolnaftate in the pharmaceutical formulations may range from about 0.25 mg/g to about 200 mg/g, from about 0.5 mg/g to about 175 mg/g, from about 0.5 mg/g to about 150 mg/g, from about 0.5 mg/g to about 100 mg/g, from about 0.5 mg/g to about 75 mg/g, from about 0.5 mg/g to about 50 mg/g, from about 0.5 mg/g to about 25 mg/g, from about 0.5 mg/g to about 20 mg/g, from about 0.5 mg/g to about 10 mg/g, from about 0.5 mg/g to about 5 mg/g, from about 0.5 mg/g to about 4 mg/g, from about 0.5 mg/g to about 3 mg/g, from about 0.5 mg/g to about 2 mg/g, or from about 0.5 mg/g to about 1.5 mg/g.

In certain embodiments, the topical formulations provided herein also comprise a polar liquid medium. In certain embodiments, the topical formulations provided herein are administered in an aqueous medium. The topical formulations provided herein may be in the form of a solution, suspension, gel, fluid gel, emulsion, emulsion gel, cream, lotion, ointment, spray, film forming solution, lacquer or a patch soaked with the formulation.

4.2.4. Grisefulvin

Griseofulvin is an antifungal having the structure of:

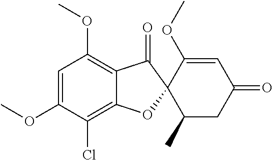

Griseofulvin may be used in the formulations provided herein in its free form, or its pharmaceutically acceptable solvate, hydrate, or salt form. In a specific embodiment, griseofulvin is used in its free form. The term “griseofulvin” as used herein includes the free form of the compound as well as pharmaceutically acceptable solvate, hydrate, or salt form.

The pharmaceutical formulations provided herein allow for the topical administration of griseofulvin, and comprise a therapeutically effective amount of griseofulvin and at least one lipid and at least one surfactant, wherein the formulation comprises 0.25-25% griseofulvin in terms of dry “total lipid” weight being defined as the sum total of dry weights of all included lipids, surfactants, lipophilic excipients, and griseofulvin. The formulations provided herein may also comprise 0.25 to 30% by weight of griseofulvin. In specific embodiments, the topical griseofulvin formulations may comprise from about 0.25% to about 0.5%, from about 0.5% to about 1%, from about 1% to about 1.5%, from about 1.5% to about 2%, from about 2% to about 2.5%, from about 2.5% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 12%, from about 12% to about 14%, from about 14% to about 16%, from about 16% to about 18%, from about 18% to about 20%, from about 22% to about 24%, from about 26% to about 28%, or from about 28% to about 30% by weight of griseofulvin.

The pharmaceutical formulations provided herein contain griseofulvin in an amount ranging from about 0.25 mg/g to about 200 mg/g. In certain embodiments, the amount of griseofulvin in the pharmaceutical formulations may range from about 0.25 mg/g to about 200 mg/g, from about 0.5 mg/g to about 175 mg/g, from about 0.5 mg/g to about 150 mg/g, from about 0.5 mg/g to about 100 mg/g, from about 0.5 mg/g to about 75 mg/g, from about 0.5 mg/g to about 50 mg/g, from about 0.5 mg/g to about 25 mg/g, from about 0.5 mg/g to about 20 mg/g, from about 0.5 mg/g to about 10 mg/g, from about 0.5 mg/g to about 5 mg/g, from about 0.5 mg/g to about 4 mg/g, from about 0.5 mg/g to about 3 mg/g, from about 0.5 mg/g to about 2 mg/g, or from about 0.5 mg/g to about 1.5 mg/g.

In certain embodiments, the griseofulvin formulations provided herein also comprise a polar liquid medium. In certain embodiments, the griseofulvin formulations provided herein are administered in an aqueous medium. The griseofulvin formulations provided herein may be in the form of a solution, suspension, gel, fluid gel, emulsion, emulsion gel, cream, lotion, ointment, spray, film forming solution, lacquer or a patch soaked with the formulation.

TABLE I
Antifungal Agents
ANTIFUNGAL AGENTS

5-fluorocytosine, Abafungin, Acrisorcin, Amorolfine,
Albaconazole, Albendazole, Amorolfine, Anidulafungin,
Arasertaconazole, Azithromycin, Becliconazole, Benzodithiazole,
Bifonazole, Butenafine, Butoconazole, Calbistrin, Caspofungin,
Chloroxine, Chlorphenesin, Ciclopiroxolamine, Ciclopirox,
Cioteronel, Clotrimazole, Croconazole, Cytoporins,
Deoxymulundocandin, Eberconazole, Econazole, Efungumab,
Fenticonazole, Flavanoid glycosides, Fluconazole, Flutrimazole,
Flucytosine, Fosfluconazole, Genaconazole, Gentian violet,
Griseofulvin, Griseofulvin-PEG, Haloprogin, Hydroxyitraconazole,
Isoconazole, Itraconazole, Ketoconazole, Lanoconazole, Letrazuril,
Liranaftate, Luliconazole, Micafungin, Miconazole, Mycophenolic
acid, Naftifine, N-chlorotaurine, Natamycin, Nitazoxanide, Nitro-
ethylene based antifungals, Nystatin, Omoconazole, Oxiconazole,
Polyene macrolide, Posaconazole, Pramiconazole, Quinolone analogs,
Rapamycin, Ravuconazole, Rilopirox, Samidazole, Sertaconazole,
Sitamaquine, Sordaricin, Squalestatin, a Squaline Expoxidase
Inhibitor, Sulconazole, Sultriecin, Tafenoquine, Terbinafine,
Terconazole, Tioconazole, Tolnaftate, Voriconazole

In an embodiment of the invention, the antifungal agent is not Terbinafine. In an embodiment of the invention, the antifungal agent is not Amphotericin B.

4.3. Antibacterials

Antibacterials that are suitable for use in the antibacterial formulations provided herein include, but are limited to, benzyl alcohol, methyl paraben ethanol, isopropanol, glutaraldehyde, formaldehyde, chlorine compounds, iodine compounds, hydrogen peroxide, peracetic acid, ethylene oxide, triclocarban, chlorhexidine, alexidine, triclosan, hexachlorophene, polymeric biguanides, formaldehyde, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim.

Additional, non-limiting examples of antibiotics include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, pcnamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2.4 diaminopyrimidines (e.g., brodimoprim).

Examples of antibacterials that can be used to inhibit the proliferation or viability of Mycobacterium tuberculosis include, but are not limited to Isoniazid, Rifampin, Pyrazinamide, Ethambutol, and Streptomycin.

Examples of antibacterials that can be used to inhibit the proliferation or viability of a mycoplasma include, but are not limited to, erythromycin, azithromycin, clarithromycin, tetracycline, doxycycline, minocycline, clindamycin, ofloxacin, and chloramphenicol.

4.4. Lipid

In the sense of this disclosure, a “lipid” is any substance, which has properties like or similar to those of a lat. As a rule, it has an extended apolar group (the “chain”, X) and generally also a water-soluble, polar hydrophilic part, the “head” group (Y) and has the basic Formula II:

X—Yₙ (II)

wherein n is equal to or larger than zero.

Lipids with n=0 are referred to as apolar lipids and lipids with n≧1 are referred to as polar lipids. In this sense, all amphiphilic substances, including, hut not limited to glycerides, glycerophospholipids, glycerophosphinolipids, glycerophosphonolipids, sulfolipids, sphingolipids, isoprenoid lipids, steroids or sterols and carbohydrate-containing lipids can generally be referred to as lipids, and are included as such in this disclosure. A list of relevant lipids and lipid related definitions is provided in EP 0 475 160 A1 (see, e.g. p. 4, l. 8 to p. 6, l. 3) and U.S. Pat. No. 6,165,500 (see, e.g., col. 6, l. 10 to col. 7, l. 58), which are herewith incorporated by reference.

A phospholipid is, for example, a compound of Formula III:

R¹—CH₂—CHR²—CR³H—O—PHO₂—O—R⁴ (III)

wherein R¹ and R² cannot both be hydrogen, OH or a C₁-C₃ alkyl group, and typically are independently, an aliphatic chain, most often derived from a fatty acid or a fatty alcohol; R³ generally is a hydrogen.

The OH-group of the phosphate is a hydroxyl radical or hydroxyl anion (i.e., hydroxide) form, dependent on degree of the group ionization. Furthermore, R⁴ may be a proton or a short-chain alkyl group, substituted by a tri-short-chain alkylammonium group, such as a trimethylammonium group, or an amino-substituted short-chain alkyl group, such as 2-trimethylammonium ethyl group (cholinyl) or 2-dimethylammonium short alkyl group.

A sphingophospholipid is, for example, a compound of Formula IIIB:

R¹-Sphingosine-O—PHO₂—O—R⁴ (IIIB)

wherein R¹ is a fatty-acid attached via an amide bond to the nitrogen of the sphingosine and R⁴ has the meanings given under Formula III.

A lipid preferably is a substance of formulae III or IIIB, wherein R¹ and/or R² are acyl or alkyl, n-hydroxyacyl or n-hydroxyalkyl, but may also be branched, with one or more methyl groups attached at almost any point of the chain; usually, the methyl group is near the end of the chain (iso or anteiso). The radicals R¹ and R² may moreover either be saturated or unsaturated (mono-, di- or poly-unsaturated). R³ is hydrogen and R⁴ is 2-trimethylammonium ethyl (the latter corresponds to the phosphatidyl choline head group), 2-dimethylammonium ethyl, 2-methylammonium ethyl or 2-aminoethyl (corresponding to the phosphatidyl ethanolamine head group). R⁴ may also be a proton (giving phosphatidic acid), a serine (giving phosphatidylserine), a glycerol (giving phosphatidylglycerol), inositol (giving phosphatidylinositol), or an alkylamine group (giving phosphatidylethanolamine in case of an ethylamine), if one chooses to use a naturally occurring glycerophospholipid. Otherwise, any other sufficiently polar phosphate ester, such that will form a lipid bilayer, may be considered as well for making the formulations of the disclosure.

Table 2 lists preferred phospholipids in accordance with the disclosure.

TABLE 2
Preferred (phospho)lipids for use in combination with an antimicrobial provided herein

| | | Phospholipid: Type and Charge | | | | | |
|---|---|---|---|---|---|---|---|
| Fatty chain | | Phos- | | | | | Phospha- |
| | Length: | phatidyl- | | | | | tidic |
| | nr. of | choline/± | | | | | acid/− |
| | double | Main | Phosphatidylethanolamine/± | Sphingomyelin/+ | Phosphatidylglycerol/− | Phosphatidylinositol/− | Aux. |
| Name(s) | bonds | lipid, L1 | Main lipid, L1 | Main lipid, L1 | Aux. lipid, L2 | Aux. lipid, L2 | lipid, L2 |
| | C24 | | | | | | |
| Behen(o)yl | C22 | | | | | | |
| Eruca(o)yl | C22:1- | | | | | | |
| | 13cis | | | | | | |
| Arachin(o)yl | C20 | | | | | | |
| Gadolen(o)yl | C20:1- | | | | | | |
| | 11cis | | | | | | |
| Arachidon(o)yl | C20:4- | | | | | | |
| | 5,8,11, | | | | | | |
| | 14cis | | | | | | |
| Ole(o)yl | C18:1-9cis | DOPC | DOPE | SM-oleyl | DOPG | DOPI | DOPA |
| Stear(o)yl | C18 | | | | | | |
| Linol(o)yl | C18:2- | (Soy-PC/ | (Soy-PE/ | Brain SM | (Soy-PC/ | (Soy-PI/ | (Soy-PA/ |
| | 9,12cis | | | | | | |
| Linole(n/o)yl | C18:3- | Egg-PC) | Egg-PE) | | Egg-PC) | Liver-PI) | Egg-PA) |
| | 9,12,15cis | | | | | | |
| Palmitole(o)yl | C18:1-9cis | | | | | | |
| Palmit(o)yl | C16 | | | | | | |
| Myrist(o)yl | C14 | DMPC | DMPE | SM-myristyl | DMPG | DMPI | |
| Laur(o)yl | C12 | DLPC | DLPE | SM-lauryl | | | DLPA |
| Capr(o)yl | C10 | | | | | | |

| | | | | | |
|---|---|---|---|---|---|
| Rel. concentration range | 1/0 | 1/0 | 10/1-1/1 | 10/1-3/1 | 10/1-5/1 |
| L1/L2 (M/M) | | | | | |
| “Total Lipid”* concentration | 0.5-45 | 0.5-45 | 0.5-40 | 0.5-40 | 0.5-40 |
| range (w-%) | | | | | |

*Total Lipid includes phospholipid(s), surfactant, an antifungal or an antibacterial provided herein, and all lipophilic excipients
An antifungal provided herein is incorporated in up to 15 rel. w-% into acidic formulations and up to 10 rel. w-% into neutral pH formulations

The preferred lipids in context of this disclosure are uncharged and form stable, well hydrated bilayers; phosphatidylcholines, phosphatidylethanolamine, and sphingomyelins are the most prominent representatives of such lipids. Any of those can have chains as listed in the Table 2, the ones forming fluid phase bilayers, in which lipid chains are in disordered state, being preferred.

Different negatively charged. i.e., anionic, lipids can also be incorporated into vesicular lipid bilayers to modify the (cationic) drug loading into or release from the resulting lipid aggregates. Attractive examples of such charged lipids are phosphatidylglycerols, phosphatidylinositols and, somewhat less preferred, phosphatidic acid (and its alkyl ester) or phosphatidylserine. It will be realized by anyone skilled in the art that it is less commendable to make vesicles just from the charged lipids than to use them in a combination with electro-neutral bilayer component(s). In case of using charged lipids, buffer composition and/or pH care must selected so as to ensure the desired degree of lipid head-group ionization and/or the desired degree of electrostatic interaction between the, oppositely, charged drug and lipid molecules. Moreover, as with neutral lipids, the charged bilayer lipid components can in principle have any of the chains listed in the Table 2. The chains forming fluid phase lipid bilayers are clearly preferred, however, both due to vesicle adaptability increasing role of increasing fatty chain fluidity and due to better ability of lipids in fluid phase to mix with each other, and with drugs.

The fatty acid- or fatty alcohol-derived chain of a lipid is typically selected amongst the basic aliphatic chain types given in the following tables:

TABLE 3
The (most) preferred basic, straight, saturated fatty chain residues

| Shorthand designation | Systematic name | Trivial name |
|---|---|---|
| 12:0 | Dodecanoic | Lauric |
| 13:0 | Tridecanoic | |
| 14:0 | Tetradecanoic | Myristic |
| 15:0 | Pentadecanoic | |
| 16:0 | Hexadecanoic | Palmitic |
| 17:0 | Heptadecanoic | Margaric |
| 18:0 | Octadecanoic | Stearic |
| 19:0 | Nonadecanoic | |
| 20:0 | Eicosanoic | Arachidic |
| 21:0 | Heneicosanoic | |
| 22:0 | Docosanoic | Behenic |
| 23:0 | Tricosanoic | |
| 24:0 | Tetracosanoic | Lignoceric |

TABLE 4
The (most) preferred monoenoic fatty chain residues

| Shorthand designation | Systematic name | Trivial name |
|---|---|---|
| 9-14:1/14:1(n − 5) | cis-9-Tetradecenoic | Myristoleic |
| 7-16:1/16:1(n − 9) | cis-7-Hexadecenoic | |
| 9-16:1/16:1(n − 7) | cis-9-Hexadecenoic | Palmitoleic |
| 9-18:1/18:1(n − 9) | cis-9-Octadecenoic | Oleic |
| 11-18:1/18:1(n − 7) | cis-11-Octadecenoic | cis-Vaccenic |
| 11-20:1/20:1(n − 9) | cis-11-Eicosenoic | Gondoic |
| 14-20:1/20:1(n − 6) | cis-14-Eicosaenoic | |
| 13-22:1/22:1(n − 9) | cis-13-Docosenoic | Erucic |
| 15-24:1/24:1(n − 9) | cis-15-Tetracosenoic | Nervoni |
| 3t-18:1 | trans-3-Hexadecenoi | |
| 9t-18:1 | trans-9-Octadecenoic | Elaidic |
| 11t-18:1 | trans-11-Octadecenoic | Vaccenic |

TABLE 5
The (most) preferred dienoic and polyenoic fatty chain residues

| Shorthand designation | Systematic name | Trivial name |
|---|---|---|
| 10,13c-16:2/16:2(n − 3) | 10-cis,13-cis-Hexadecadienoic | |
| 7,10c-16:2/16:3(n − 6) | 7-cis,10-cis-Hexadecadienoic | |
| 7,10,13c-16:3/16:3(n − 3) | 7-cis,10-cis,13-cis-Hexadecatrienoic | |
| 12,15c-18:2/18:2(n − 3) | 12-cis,15-cis-Octadecadienoic | α-Linoleic |
| 10,12t-18:2/18:2(n − 6) | trans-10,trans-12-Octadecadienoic | |
| 9,12c-18:2/18:2(n − 6) | 9-cis,12-cis-Octadecadienoic | γ-Linoleic |
| 9,12,15c-18:3/18:3(n − 3) | 9-cis,12-cis,15-cis-Octadecatrienoic | α-Linolenic |
| 6,9,12c-18:3/18:3(n − 6) | 6-cis,9-cis,12-cis-Octadecatrienoic | γ-Linolenic |
| 9c,11c,13t-18:3 | 9-cis,11-trans,13-trans-Octadecatrienoic | α-Eleostearic |
| 8t,10t,12c-18:3 | 8-trans,10-trans,12-cis-Octadecatrienoic | Calendic |
| 6,9,12,15c-18:4/18:4(n − 3) | 6,9,12,15-Octadecatetraenoic | Stearidonic |
| 3,6,9,12c-18:4/18:4(n − 6) | 3,6,9,12-Octadecatetraenoic | |
| 3,6,9,12,15c-18:5/18:5(n − 3) | 3,6,9,12,15-Octadecapentaenoic | |
| 14,17c-20:2/20:2(n − 3) | 14-cis,17-cis-Eicosadienoic | |
| 11,14c-20:2/20:2(n − 6) | 11-cis,14-cis-Eicosadienoic | |
| 11,14,17c-20:3/20:3(n − 3) | 8-cis,11-cis,14-cis-Eicosatrienoic | Dihomo-α-linolenic |
| 8,11,14c-20:3/20:3(n − 6) | 8-cis,11-cis,14-cis-Eicosatrienoic | Dihomo-γ-linolenic |
| 5,8,11c-20:3 20:3(n − 9) | 5,8,11all-cis-Eicosatrienoic | ‘Mead's’ |
| 5,8,11,14c-20:4/20:4(n − 6) | 5,8,11; 14-all-cis-Eicosatetraenoic | Arachidonic |
| 8,11,14,17c-20:4/20:4(n − 3) | 8,11,14,17-all-cis-Eicosatetraenoic | |
| 5,8,11,14,17c-20:5 | 5,8,11,14,17-all-cis-Eicosapentaenoic | |
| or 20:5(n − 3) | | |
| 13,16c-22:2 | 13,16-Docosadienoic | |
| 13,16,19c-22:3/22:3(n − 3) | 13,16,19-Docosatrienoic | |
| 10,13,16c-22:3/22:3(n − 6) | 10,13,16-Docosatrienoic | |
| 7,10,13,16c-22:4/22:4(n − 6) | 7,10,13,16-Docosatetraenoic | Adrenic |
| 4,7,10,13,16c-22:5 | 4,7,10,13,16-Docosapentaenoic | |
| or 22:5(n − 6) | | |
| 4,7,10,13,16,19c-22:5 | 4,7,10,13,16,19-Docosahexaenoic | |
| or 22:6(n − 3) | | |

Other double bond combinations or positions are possible as well.

Suitable fatty residues can furthermore be branched, for example, can contain a methyl group in an iso or anteiso position of the fatty acid chain, or else closer to the chain middle, as in 10-R-methyloctadecanoic acid or tuberculostearic chain. Relatively important amongst branched fatty acids are also isoprenoids, many of which are derived from 3,7,11,15-tetramethylhexadec-trans-2-en-1-ol, the aliphatic alcohol moiety of chlorophyll. Examples include 5,9,13,17-tetramethyloctadecanoic acid and especially 3,7,11,15-tetramethylhexadecanoic (phytanic) and 2,6,10,14-tetramethylpentadecanoic (pristanic) acids. A good source of 4,8,12-trimethyltridecanoic acid are marine organisms. Combination of double bonds and side chains on a fatty residue are also possible.

Alternatively, suitable fatty residues may carry one or a few oxy- or cyclic groups, especially in the middle or towards the end of a chain. The most prominent amongst the later, alicyclic fatty acids, are those comprising a cyclopropane (and sometimes cyclopropene) ring, but cyclohexyl and cycloheptyl rings can also be found and might be useful for purposes of this disclosure. 2-(D)-Hydroxy fatty acids are more ubiquitous than alicyclic fatty acids, and are also important constituents of sphingolipids. Also interesting are 15-hydroxy-hexadecanoic and 17-hydroxy-octadecanoic acids, and may be 9-hydroxy-octadeca-trans-10,trans-12-dienoic (dimorphecolic) and 13-hydroxy-octadeca-cis-9,trans-11-dienoic (coriolic) acid. Arguably the most prominent hydroxyl-fatty acid in current pharmaceutical use is ricinoleic acid, (D-(−)12-hydroxy-octadec-cis-9-enoic acid, which comprises up to 90% of castor oil, which is also often used in hydrogenated form. Epoxy-, methoxy-, and furanoid-fatty acids are of only limited practical interest in the context of this disclosure.

Generally speaking, unsaturation, branching or any other kind of derivatization of a fatty acid is best compatible with the intention of present disclosure of the site of such modification is in the middle or terminal part of a fatty acid chain. The cis-unsaturated fatty acids are also more preferable than trans-unsaturated fatty acids and the fatty radicals with fewer double bonds are preferred over those with multiple double bonds, due to oxidation sensitivity of the latter. Moreover, symmetric chain lipids are generally better suited than asymmetric chain lipids.

A preferred lipid of the Formula III is, for example, a natural phosphatidylcholine, which used to be called lecithin. It can be obtained from egg (rich in palmitic, C_{16:0}, and oleic, C_{18:1}, but also comprising stearic, C_{18:0}, palmitoleic, linolenic, C_{18:2}, and arachidonic, C_{20:4}, radicals), soybean (rich in unsaturated C₁₈ chains, but also containing some palmitic radical, amongst a few others), coconut (rich in saturated chains), olives (rich in monounsaturated chains), saffron (safflower) and sunflowers (rich in n−6 linoleic acid), linseed (rich in n−3 linolenic acid), from whale fat (rich in monounsaturated n−3 chains), from primrose or primula (rich in n−3 chains). Preferred, natural phosphatidyl ethanolamines (used to be called cephalins) frequently originate from egg or soybeans. Preferred sphingomyelins of biological origin are typically prepared from eggs or brain tissue. Preferred phosphatidylserines also typically originate from brain material whereas phosphatidylglycerol is preferentially extracted from bacteria, such as E. Coli, or else prepared by way of transphosphatidylation, using phospholipase D, starting with a natural phosphatidylcholine. The preferably used phosphatidylinositols are isolated from commercial soybean phospholipids or bovine liver extracts. The preferred phosphatidic acid is either extracted from any of the mentioned sources or prepared using phospholipase D from a suitable phosphatidylcholine.

Furthermore, synthetic phosphatidyl cholines (R⁴ in Formula III corresponds to 2-trimethylammonium ethyl), and R¹ and R² are aliphatic chains, as defined in the preceding paragraph with 12 to 30 carbon atoms, preferentially with 14 to 22 carbon atoms, and even more preferred with 16 to 20 carbon atoms, under the proviso that the chains must be chosen so as to ensure that the resulting ESAs comprise fluid lipid bilayers. This typically means use of relatively short saturated and of relatively longer unsaturated chains. Synthetic sphingomyelins (R⁴ in Formula IIIB corresponds to 2-trimethylammonium ethyl), and R¹ is an aliphatic chain, as defined in the preceding paragraph, with 10 to 20 carbon atoms, preferentially with 10 to 14 carbon atoms per fully saturated chain and with 16-20 carbon atoms per unsaturated chain.

Synthetic phosphatidyl ethanolamines (R⁴ is 2-aminoethyl), synthetic phosphatidic acids (R⁴ is a proton) or its ester (R⁴ corresponds, for example, to a short-chain alkyl, such as methyl or ethyl), synthetic phosphatidyl serines (R⁴ is L- or D-serine), or synthetic phosphatidyl (poly)alcohols, such as phosphatidyl inositol, phosphatidyl glycerol (R⁴ is L- or D-glycerol) are preferred as lipids, wherein R¹ and R² are fatty residues of identical or moderately different type and length, especially such as given in the corresponding tables given before in the text. Moreover, R¹ can represent alkenyl and R² identical hydroxyalkyl groups, such as tetradecylhydroxy, or hexadecylhydroxy, for example, in ditetradecyl or dihexadecylphosphatidyl choline or ethanolamine, R¹ can represent alkenyl and R² hydroxyacyl, such as a plasmalogen (R⁴ trimethylammonium ethyl), or R¹ can be acyl, such as lauryl, myristoyl or palmitoyl and R² can represent hydroxy as, for example, in natural or synthetic lysophosphatidyl cholines or lysophosphatidyl glycerols or lysophosphatidyl ethanolamines, such as 1-myristoyl or 1-palmitoyllysophosphatidyl choline or -phosphatidyl ethanolamine; frequently, R³ represents hydrogen.

A lipid of Formula IIIB is also a suitable lipid within the sense of this disclosure. In Formula IIIB, n=1, R¹ is an alkenyl group, R² is an acylamido group, R³ is hydrogen and R⁴ represents 2-trimethylammonium ethyl (choline group). Such a lipid is known under the name of sphingomyelin.

Suitable lipids furthermore are a lysophosphatidyl choline analog, such as 1-lauroyl-1,3-dihydroxypropane-3-phosphoryl choline, a monoglyceride, such as monoolein or monomyristin, a cerebroside, ceramide polyhexoside, sulfatide, sphingoplasmalogen, a ganglioside or a glyceride, which does not contain a free or esterified phosphoryl or phosphono or phosphino group in the 3 position. An example of such a glyceride is diacylglyceride or 1-alkenyl-1-hydroxy-2-acyl glyceride with any acyl or alkenyl groups, wherein the 3-hydroxy group is etherified by one of the carbohydrate groups named, for example, by a galactosyl group such as a monogalactosyl glycerin.

Lipids with desirable head or chain group properties can also be formed by biochemical means, for example, by means of phospholipases (such as phospholilpase A1, A2, B, C and, in particular, D), desaturases, elongases, acyl transferases, etc., from natural or synthetic precursors.

Furthermore, a suitable lipid is any lipid, which is contained in biological membranes and can be extracted with the help of apolar organic solvents, such as chloroform. Aside from the lipids already mentioned, such lipids also include, for example, steroids, such as estradiol, or sterols, such as cholesterol, beta-sitosterol, desmosterol, 7-keto-cholesterol or beta-cholestanol, fat-soluble vitamins, such as retinoids, vitamins, such as vitamin A1 or A2, vitamin E, vitamin K, such as vitamin K1 or K2 or vitamin D1 or D3, etc.

The less soluble amphiphilic components comprise or preferably comprise a synthetic lipid, such as myristoleoyl, palmitoleoyl, petroselinyl, petroselaidyl, oleoyl, elaidyl, cis- or trans-vaccenoyl, linolyl, linolenyl, linolaidyl, octadecatetraenoyl, gondoyl, eicosaenoyl, eicosadienoyl, eicosatrienoyl, arachidoyl, cis- or trans-docosaenoyl, docosadienoyl, docosatricnoyl, docosatetraenoyl, lauroyl, tridecanoyl, myristoyl, pentadecanoyl, palmitoyl, heptadecanoyl, stearoyl or nonadecanoyl, glycerophospholipid or corresponding derivatives with branched chains or a corresponding dialkyl or sphingosin derivative, glycolipid or other diacyl or dialkyl lipid.

The more soluble amphiphilic components(s) is/are frequently derived from the less soluble components listed above and, to increase the solubility, substituted and/or complexed and/or associated with a butanoyl, pentanoyl, hexanoyl, heptanoyl, octanoyl, nonanoyl, decanoyl or undecanoyl substituent or several, mutually independent, selected substituents or with a different material for improving the solubility.

A further suitable lipid is a diacyl- or dialkyl-glycerophosphoetha-nolamine azo polyethoxylene derivative, a didecanoylphosphatidyl choline or a diacylphosphoolligomaltobionamide.

In certain embodiments, the amount of lipid in the formulation is from about 1% to about 30%, about 1% to about 10%, about 1% to about 4%, about 4% to about 7% or about 7% to about 10% by weight. In a specific, embodiment, the lipid is a phospholipid. In another specific embodiment, the phospholipid is a phosphatidylcholine. In one embodiment, the formulations provided herein contain an antifungal or an antibacterial, phosphatidylcholine, and a surfactant, wherein the formulation contains 1-10% by weight of phosphatidylcholine.

4.5. Surfactant

The term “surfactant” has its usual meaning. A list of relevant surfactants and surfactant related definitions is provided in EP 0 475 160 A 1 (see, e.g., p. 6, l. 5 to p. 14, l. 17) and U.S. Pat. No. 6,165,500 (see, e.g., col. 7, l. 60 to col. 19, l. 64) which are herewith incorporated by reference, and in appropriate surfactant or pharmaceutical Handbooks, such as Handbook of Industrial Surfactants or US Pharmacopoeia, Pharm. Fu. In some embodiments, the surfactants are those described in Tables 1-18 of U.S. Patent Application Publication No. 2002/0012680 A1, published Jan. 31, 2002, the disclosure of which is hereby incorporated by reference in its entirety. The following list therefore only offers a selection, which is by no means complete or exclusive, of several surfactant classes that are particularly common or useful in conjunction with present patent application. Preferred surfactants to be used in accordance with the disclosure include those with an HLB (hydrophile—lipophile balance) greater than 12. The list includes ionized long-chain fatty acids or long chain fatty alcohols, long chain fatty ammonium salts, such as alkyl- or alkenoyl-trimethyl-, -dimethyl- and -methyl-ammonium salts, alkyl- or alkenoyl-sulphate salts, long fatty chain dimethyl-aminoxides, such as alkyl- or alkenoyl-dimethyl-aminoxides, long fatty chain, for example alkanoyl, dimethyl-aminoxides and especially dodecyl dimethyl-aminoxide, long fatty chain, for example alkyl-N-methylglucamide-s and alkanoyl-N-methylglucamides, such as MEGA-8. MEGA-9 and MEGA-10, N-long fatty chain-N,N-dimethylglycines, for example N-alkyl-N,N-dimethylglycines, 3-(long fatty chain-dimethylammonio)-alkane-sulphonates, for example 3-(acyidimethylammonio)-alkanesulphonates, long fatty chain derivatives of sulphosuccinate salts, such as bis(2-ethylalkyl) sulphosuccinate salts, long fatty chain-sulphobetaines, for example acyl-sulphobetaines, long fatty chain betaines, such as EMPIGEN BB or ZWITTERGENT-3-16, -3-14, -3-12, -3-10, or -3-8, or polyethylen-glycol-acylphenyl ethers, especially nonaethylen-glycol-octyl-phenyl ether, polyethylene-long fatty chain-ethers, especially polyethylene-acyl ethers, such as nonaethylen-decyl ether, nonaethylen-dodecyl ether or octaethylene-dodecyl ether, polyethyleneglycol-isoacyl ethers, such as octaethyleneglycol-isotridecyl ether, polyethyleneglycol-sorbitane-long fatty chain esters, for example polyethyleneglycol-sorbitane-acyl esters and especially polyoxyethylene-monolaurate (e.g. polysorbate 20 or Tween 20), polyoxyethylene-sorbitan-monooleate (e.g. polysorbate 80 or Tween 80), polyoxyethylene-sorbitan-monolauroleylate, polyoxyethylene-sorbitan-monopetroselinate, polyoxyethylene-sorbitan-monoelaidate, polyoxyethylene-sorbitan-myristoleylate, polyoxyethylene-sorbitan-palmitoleinylate, poly oxyethylene-sorbitan-p-etroselinylate, polyhydroxyethylene-long fatty, chain ethers, for example polyhydroxyethylene-acyl ethers, such as polyhydroxyethylene-lauryl ethers, polyhydroxyethylene-myristoyl ethers, polyhydroxyethylene-cetylst-earyl, polyhydroxyethylene-palmityl ethers, polyhydroxyethylene-oleoyl ethers, polyhydroxyethylene-palmitoleoyl ethers, polyhydroxyethylene-lino-leyl, polyhydroxyethylen-4, or 6, or 8, or 10, or 12-lauryl, miristoyl, palmitoyl, palmitoleyl, oleoyl or linoeyl ethers (Brij series), or in the corresponding esters, polyhydroxyethylen-laurate, -myristate, -palmitate, -stearate or -oleate, especially polyhydroxyethylen-8-stearate (Myrj 45) and polyhydroxyethylen-8-oleate, polyethoxylated castor oil 40 (Cremophor EL), sorbitane-mono long fatty chain, for example alkylate (Arlacel or Span series), especially as sorbitane-monolaurate (Arlacel 20, Span 20), long fatty chain, for example acyl-N-methylglucamides, alkanoyl-N-methylglucamides, especially decanoyl-N-methylglucamide, dodecanoyl-N-methylglucamide, long fatty chain sulphates, for example alkyl-sulphates, alkyl sulphate salts, such as lauryl-sulphate (SDS), oleoyl-sulphate; long fatty chain thioglucosides, such as alkylthioglucosides and especially heptyl-, octyl- and nonyl-beta-D-thioglucopyranoside; long fatty chain derivatives of various carbohydrates, such as pentoses, hexoses and disaccharides, especially alkyl-glucosides and maltosides, such as hexyl-, heptyl-, octyl-, nonyl- and decyl-beta-D-glucopyranoside or D-maltopyranoside; further a salt, especially a sodium salt, of cholate, deoxycholate, glycocholate, glycodeoxycholate, taurodeoxycholate, taurocholate, a fatty acid salt, especially oleate, elaidate, linoleate, laurate, or myristate, most often in sodium form, lysophospholipids, n-octadecylene-glycerophosphatidic acid, octadecylene-phosphorylglycerol, octadceylene-phosphorylserine, n-long fatty chain-glycero-phosphatidic acids, such as n-acyl-glycero-phosphatidic acids, especially lauryl glycero-phosphatidic acids, oleoyl-glycero-phosphatidic acid, n-long fatty chain-phosphorylglycerol, such as n-acyl-phosphorylglycerol, especially lauryl-, myristoyl-, oleoyl- or palmitoeloyl-phosphorylglycerol, n-long fatty chain-phosphorylserine, such as n-acyl-phosphorylserine, especially lauryl-, myristoyl-, oleoyl- or palmitoeloyl-phosphorylserine, n-tetradecyl-glycero-phosphatidic acid, n-tetradecyl-phosphorylglycerol, n-tetradecyl-phosphorylserine, corresponding-, elaidoyl-, vaccenyl-lysophospholipids, corresponding short-chain phospholipids, as well as all surface active and thus membrane destabilising polypeptides. Surfactant chains are typically chosen to be in a fluid state or at least to be compatible with the maintenance of fluid-chain state in carrier aggregates.

Table 6 lists preferred surfactants in accordance with the disclosure.

TABLE 6
Preferred nonionic surfactants for use in combination with an antimicrobial
provided herein

| | | Nonionic surfactants (S) | | | | |
|---|---|---|---|---|---|---|
| | | Head/Type/TM | | | | |
| Fatty chain | | | POE- | POE- | POE- | |
| | Length: | POE-sorbitan- | ether | ester | phenoxy- | |
| | nr. of double | ester | Brij, | Myrj, | ether | Selected |
| Name(s) | bonds | Tween | Macrogol | Nonex | Triton | brandnames |
| | C24 | | | | | |
| Behen(o)yl | C22 | | | | | |
| Eruca(o)yl | C22:1-13cis | | | | | |
| Arachin(o)yl | C20 | | | | | |
| Gadolen(o)yl | C20:1-11cis | | | | | |
| Arachidon(o)yl | C20:4-5,8,11,14cis | | | | | |
| Ole(o)yl | C18:1-9cis | Tween 80 | Brij 98 | Simulsol- | TritonX100** | |
| | | | | 2599 | | |
| Stear(o)yl | C18 | Tween 60 | | Myrj-52 | | |
| Linol(o)yl | C18:2-9,12cis | | | | | |
| Linole(n/o)yl | C18:3-9,12,15cis | | | | | |
| Palmitole(o)yl | C18:1-9cis | | | | | |
| Palmit(o)yl | C16 | Tween 40 | | NN | | |
| Myrist(o)yl | C14 | | | | | |
| Laur(o)yl | C12 | Tween 20 | Brij 35 | NN | | |
| Capr(o)yl | C10 | | | | | |

| | | | | |
|---|---|---|---|---|
| Rel. concentration range L/S (M/M) | 5/1-1/1 | 5/1-1/1 | 5/1-1/1 | 4/1-3/2 |

NN: not readily available in the market but in principle suitable
**Triton is not an oleate, but an octylphenoxy-POE derivative
Myrj-45: Stearoyl-EO8;
Myrj-49: Stearoyl-EO20 (not in the market);
Myrj-59: Stearoyl-EO100;
Myrj-52: Stearoyl-EO40;
Simulsol-2599 = Macrogol-10-oleate
Brij-98: Oleoyl-EO20
Brij-35: Lauryl-EO23

In certain embodiments, the surfactant is a nonionic surfactant. The surfactant may be present in the formulation in about 1% to about 50%, about 1% to about 10%, about 1% to about 4%, about 4% to about 7% or about 7% to about 10% by weight. In certain embodiments, the nonionic surfactant is selected from the group consisting of: polyoxyethylene sorbitans (polysobate surfactants), polyhydroxyethylene stearates or polyhydroxyethylene laurylethers (Brij surfactants). In a specific embodiment, the surfactant is a polyoxyethylene-sorbitan-monooleate (e.g. polysorbate 80 or Tween 80). In certain embodiments, the polysorbate can have any chain with 12 to 20 carbon atoms. In certain embodiments, the polysorbate is fluid in the formulation, which may contain one or more double bonds, branching, or cyclo-groups.

4.6. Formulations

The formulations provided herein may contain 1 to 10% by weight, 1 to 15% by weight, 1 to 20% by weight, or 1 to 30% of an antimicrobial provided herein by weight. The formulations provided herein may contain 1 to 10% by weight, 1 to 15% by weight, 1 to 20% by weight, or 1 to 30% by weight of the lipid. The formulations provided herein may contain 1 to 10% by weight, 1 to 15% by weight, 1 to 20% by weight, 1 to 30% surfactant by weight, 1 to 40% by weight, or 1 to 50% by weight.

Examples of lipid based formulations that can be used in the methods described herein include, but are not limited to, emulsions, nanoemulsions, vesicles, liposomes, micelles, microspheres, nanospheres, emulsions, lipid discs, and non-specific lipid conglomerates.

In a specific embodiment, the formulation is an ultra-deformable sub microscopic vesicle. Each vesicular carrier overcomes the skin barrier spontaneously, to deposit the drug into deep tissues, as it is drawn from the dry surface to the water-rich region beneath the skin. When applied to the skin, the carrier searches and exploits hydrophilic pathways or “pores” between the cells in the skin, which it opens wide enough to permit the entire vesicle to pass through together with its drug cargo, deforming itself extremely to accomplish this without losing its vesicular integrity or releasing its cargo. The carrier then avoids the local microvasculature in order to deposit the drug at various depths in or below the skin, where the active ingredient is preferentially and slowly released to its targeted tissue.

The formulations provided herein may have a range of lipid to surfactant ratios. The ratios may be expressed in terms of molar terms (mol lipid/mol surfactant). The molar ratio of lipid to surfactant in the formulations provided herein may be from about 1:2 to about 10:1. In certain embodiments, the ratio is from about 1:1 to about 2:1, from about 2:1 to about 3:1, from about 3:1 to about 4:1, from about 4:1 to about 5:1, or from about 5:1 to about 10:1. In specific embodiments, the lipid to surfactant ratio is about 1.0, about 1.25, about 1.5, about 1.75, about 2.0, about 2.5, about 3.0, or about 4.0.

The formulations provided herein may have varying ratios of the antimicrobial to lipid. The ratios may be expressed in terms of molar ratios (mol antifungal/mol lipid). The molar ratio of the antimicrobial to lipid in the formulations provided herein may be from about 1:50 to about 50:1, from about 1:25 to about 25:1, from about 1:10 to about 10:1, from about 1:5 to about 5:1, from about 1:50 to about 50:1, or from about 0.2:1 to about 2:1. In certain embodiments, the ratio is from about 0.2:1 to about 0.7:1, from about 0.7:1 to about 1.2:1, from about 1.2:1 to about 1.7:1, or from about 1.7:1 to about 2:1.

The formulations provided herein may also have varying amounts of total amount of the following three components: the antimicrobial, lipid and surfactant combined (TA). The TA amount may be stated in terms of weight percent of the total composition. In one embodiment, the TA is from about 1% to about 40%, about 5% to about 30%, about 7.5% to about 15%, about 5% to about 10%, about 10% to about 20%, or about 20% to about 30%. In specific embodiments, the TA is 8%, 9%, 10%, 15%, or 20%.

Selected ranges for total lipid amounts, lipid/surfactant ratios (mol/mol) and the antimicrobial/surfactant ratios (mol/mol) for antimicrobial formulations provided herein are described in Table 7 below:

TABLE 7
Total Lipid, Lipid to Surfactant Ratios
and Antimicrobial to Lipid Ratios

| TA (antimicrobial, lipid | Lipid/Surfactant | Antimicrobial/Lipid |
|---|---|---|
| and surfactant) (%) | (mol/mol) | (mol/mol) |
| 5 to 10 | 1.0 to 1.25 | 0.20 to 0.75 |
| 5 to 10 | 1.0 to 1.25 | 0.75 to 1.25 |
| 5 to 10 | 1.0 to 1.25 | 1.25 to 2.00 |
| 5 to 10 | 1.25 to 1.75 | 0.20 to 0.75 |
| 5 to 10 | 1.25 to 1.75 | 0.75 to 1.25 |
| 5 to 10 | 1.25 to 1.75 | 1.25 to 2.00 |
| 5 to 10 | 1.75 to 2.25 | 0.20 to 0.75 |
| 5 to 10 | 1.75 to 2.25 | 0.75 to 1.25 |
| 5 to 10 | 1.75 to 2.25 | 1.25 to 2.00 |
| 5 to 10 | 2.25 to 3.00 | 0.20 to 0.75 |
| 5 to 10 | 2.25 to 3.00 | 0.75 to 1.25 |
| 5 to 10 | 2.25 to 3.00 | 1.25 to 2.00 |
| 5 to 10 | 2.25 to 3.00 | 2.00 to 2.25 |
| 5 to 10 | 3.00 to 4.00 | 0.20 to 0.75 |
| 5 to 10 | 3.00 to 4.00 | 0.75 to 1.25 |
| 5 to 10 | 3.00 to 4.00 | 1.25 to 2.00 |
| 5 to 10 | 3.00 to 4.00 | 2.00 to 2.25 |
| 10 to 20 | 1.0 to 1.25 | 0.20 to 0.75 |
| 10 to 20 | 1.0 to 1.25 | 0.75 to 1.25 |
| 10 to 20 | 1.0 to 1.25 | 1.25 to 2.00 |
| 10 to 20 | 1.25 to 1.75 | 0.20 to 0.75 |
| 10 to 20 | 1.25 to 1.75 | 0.75 to 1.25 |
| 10 to 20 | 1.25 to 1.75 | 1.25 to 2.00 |
| 10 to 20 | 1.75 to 2.25 | 0.20 to 0.75 |
| 10 to 20 | 1.75 to 2.25 | 0.75 to 1.25 |
| 10 to 20 | 1.75 to 2.25 | 1.25 to 2.00 |
| 10 to 20 | 2.25 to 3.00 | 0.20 to 0.75 |
| 10 to 20 | 2.25 to 3.00 | 0.75 to 1.25 |
| 10 to 20 | 2.25 to 3.00 | 1.25 to 2.00 |
| 10 to 20 | 2.25 to 3.00 | 2.00 to 2.50 |
| 10 to 20 | 3.00 to 4.00 | 0.20 to 0.75 |
| 10 to 20 | 3.00 to 4.00 | 0.75 to 1.25 |
| 10 to 20 | 3.00 to 4.00 | 1.25 to 2.00 |
| 10 to 20 | 3.00 to 4.00 | 2.00 to 2.50 |

The formulations provided herein may optionally contain one or more of the following ingredients: co-solvents, chelators, buffers, antioxidants, preservatives, microbicides, emollients, humectants, lubricants, and thickeners. Preferred amounts of optional components are described in Table 8.

The formulations provided herein may include a buffer to adjust the pH of the aqueous solution to a range from pH 3.5 to pH 9.5, pH 4 to pH 7.5, or pH 4 to pH 6.5. Examples of buffers include, but are not limited to acetate buffers, lactate buffers, phosphate buffers, and propionate buffers.

The formulations provided herein are typically formulated in aqueous media. The formulations may be formulated with or without co-solvents, such as lower alcohols.

A “microbicide” or “antimicrobial” agent is commonly added to reduce the bacterial count in pharmaceutical formulations. Some examples of microbicides are short chain alcohols, including ethyl and isopropyl alcohol, chlorbutanol, benzyl alcohol, chlorbenzyl alcohol, dichlorbenzylalcohol, hexachlorophene; phenolic compounds, such as cresol, 4-chloro-m-cresol, p-chloro-m-xylenol, dichlorophene, hexachlorophene, povidon-iodine; parabenes, especially alkyl-parabenes, such as methyl-, ethyl-, propyl-, or butyl-paraben, benzyl paraben; acids, such as sorbic acid, benzoic acid and their salts; quaternary ammonium compounds, such as alkonium salts, e.g., a bromide, benzalkonium salts, such as a chloride or a bromide, cetrimonium salts, e.g., a bromide, phenoalkecinium salts, such as phenododecinium bromide, cetylpyridinium chloride and other salts; furthermore, mercurial compounds, such as phenylmercuric acetate, borate, or nitrate, thiomersal, chlorhexidine or its gluconate, or any antibiotically active compounds of biological origin, or any suitable mixture thereof.

Examples of “antioxidants” are butylated hydroxyanisol (BHA), butylated hydroxytoluene (BHT) and di-tert-butylphenol (LY178002, LY256548, HWA-131, BF-389, CI-986, PD-127443, E-5119, BI-L-239XX, etc.), tertiary butylhydroquinone (TBHQ), propyl gallate (PG), 1-O-hexyl-2,3,5-trimethylhydroquinone (HTHQ); aromatic amines (diphenylamine, p-alkylthio-o-anisidine, ethylenediamine derivatives, carbazol, tetrahydroindenoindol); phenols and phenolic acids (guaiacol, hydroquinone, vanillin, gallic acids and their esters, protocatechuic acid, quinic acid, syringic acid, ellagic acid, salicylic acid, nordihydroguaiaretic acid (NDGA), eugenol); tocopherols (including tocopherols (alpha, beta, gamma, delta) and their derivatives, such as tocopheryl-acylate (e.g. -acetate, -laurate, myristate, -palmitate, -oleate, -linoleate, etc., or any other suitable tocopheryl-lipoate), tocopheryl-polyoxyethylene-succinate; trolox and corresponding amide and thiocarboxamide analogues; ascorbic acid and its salts, isoascorbate, (2 or 3 or 6)-o-alkylascorbic acids, ascorbyl esters (e.g. 6-o-lauroyl, myristoyl, palmitoyl-, oleoyl, or linoleoyl-L-ascorbic acid, etc.). Also useful are the preferentially, oxidized compounds, such as sodium bisulphite, sodium metabisulphite, thiourea; chellating agents, such as ethylene glycol-bis-(2-aminoethyl)-N,N,N′,N′-tetraacetic acid (EDTA), ethylenedioxy-diethylene-dinitrilo-tetraacetic acid (GDTA), desferral; miscellaneous endogenous defence systems, such as transferrin, lactoferrin, ferritin, cearuloplasmin, haptoglobion, heamopexin, albumin, glucose, ubiquinol-10); enzymatic antioxidants, such as superoxide dismutase and metal complexes with a similar activity, including catalase, glutathione peroxidase, and less complex molecules, such as beta-carotene, bilirubin, uric acid; flavonoids (flavones, flavonols, flavonones, flavanonals, chacones, anthocyanins), N-acetylcystein, mesna, glutathione, thiohistidine derivatives, triazoles; tannines, cinnamic acid, hydroxycinnamatic acids and their esters (coumaric acids and esters, caffeic acid and their esters, ferulic acid, (iso-) chlorogenic acid, sinapic acid); spice extracts (e.g., from clove, cinnamon, sage, rosemary, mace, oregano, allspice, nutmeg); carnosic acid, carnosol, carsolic acid; rosmarinic acid, rosmaridiphenol, gentisic acid, ferulic acid; oat flour extracts, such as avenanthramide 1 and 2; thioethers, dithioethers, sulphoxides, tetralkylthiuram disulphides; phytic acid, steroid derivatives (e.g., U74006F); tryptophan metabolites (e.g., 3-hydroxykynurenine, 3-hydroxyanthranilic acid), and organochalcogenides.

“Thickeners” are used to increase the viscosity of pharmaceutical formulations to and may be selected from selected from pharmaceutically acceptable hydrophilic polymers, such as partially etherified cellulose derivatives, comprising carboxymethyl-, hydroxyethyl-, hydroxypropyl-, hydroxypropylmethyl- or methyl-cellulose; completely synthetic hydrophilic polymers comprising polyacrylates, polymethacrylates, poly(hydroxyethyl)-, poly(hydroxypropyl)-, poly(hydroxypropylmethyl)methacrylate, polyacrylonitrile, methallyl-sulphonate, polyethylenes, polyoxiethylenes, polyethylene glycols, polyethylene glycol-lactide, polyethylene glycol-diacrylate, polyvinylpyrrolidone, polyvinyl alcohols, poly(propylmethacrylamide), poly(propylene fumarate-co-ethylene glycol), poloxamers, polyaspartamide, (hydrazine cross-linked) hyaluronic acid, silicone; natural gums comprising alginates, carrageenan, guar-gum, gelatine, tragacanth, (amidated) pectin, xanthan, chitosan collagen, agarose; mixtures and further derivatives or co-polymers thereof and/or other pharmaceutically, or at least biologically, acceptable polymers

The formulations provided herein may also comprise a polar liquid medium. The formulations provided herein may be administered in an aqueous medium. The formulations provided herein may be in the form of a solution, suspension, emulsion, cream, lotion, ointment, gel, spray, film forming solution or lacquer.

In one embodiment, the disclosure specifically relates to the use of an antimicrobial as provided herein, a phospholipid, and a nonionic surfactant for the preparation of a pharmaceutical composition for treating a fungal or bacterial infection, respectively. In this context, the disclosure relates to a formulation or pharmaceutical composition comprising an antimicrobial provided herein for the treatment of a fungal or bacterial infection, wherein the formulation or pharmaceutical composition is formulated for topical delivery. In one embodiment the fungal infection is not onchymycosis.

Table 8 lists preferred excipients for the formulation.

TABLE 8
Preferred excipients for use in combinations with an
antimicrobialantimicrobial provided herein
Designated activity

| | Molar (M) | | | | |
|---|---|---|---|---|---|
| | or | | Molar (M) | | |
| | Rel. | | or | | |
| Antioxydant | w %* | Antibiotic | Weight-% | Buffer | Molar |
| Primary | | | | | |
| Butylated hydroxyanisole, BHA | 0.1-8 | Acetate | 30-150 mM | Acetate | 30-150 mM |
| Butylated hydroxytoluene, BHT | 0.1-4 | Benzyl alcohol | 0.1-3 | Phosphate | 10-50 mM |
| Thymol | 0.1-1 | Butylparabene | 0.1-3 | Triethanolamine•HCL | 30-150 mM |
| Metabisulphite (MW = 190.1) | 1-5 mM | Ethylparabene | 0.1-3 | | |
| Bisulphite | 1-5 mM | Imidurea (MW = 388.30) | 0.1-1 | | |
| Thiourea (MW = 76.12) | 1-10 mM | Dimethoxane (MW = | 0.03-0.1 | | |
| Monothioglycerol (MW = 108.16) | 1-20 mM | 174.2) | | | |
| Propyl gallate (MW = 212.2) | 0.02-0.2 | Methylparabene | 0.1-5 | | |
| Ascorbate (MW = 175.3+ ion) | 1-10 mM | Phenoxyethanol | 0.1-5 | | |
| Palmityl-ascorbate | 0.01-1 | Benzalkonium chloride | 0.01-0.2 | | |
| Tocopherol-PEG | 0.5-5 | Benzethonium chloride | 0.01-0.1 | | |
| Secondary (chelator) | | Phenol | 0.05-2 | | |
| EDTA (MW = 292) | 1-10 mM | Phenylethyl alcohol | 0.1-1 | | |
| EGTA (MW = 380.35) | 1-10 mM | Thimerosal | 0.005-0.1 | | |
| Desferal (MW = 656.79) | 0.1-5 mM | | | | |

*As percentage of Total Lipid quantity
EGTA = Ethylene glycol-bis-(2-aminoethyl)-N,N,N′,N′-tetraacetic acid
EDTA = Ethylenedioxy-diethylene-dinitrilo-tetraacetic acid

4.7. Vesicular Formulations

While not to be limited to any mechanism of action or any theory, the formulations provided herein may form vesicles or ESAs characterized by their adaptability, deformability, or penetrability.

The term vesicle or aggregate “adaptability” which governs the “tolerable surface curvature” is defined as the ability of a given vesicle or aggregate to change easily its properties, such as shape, elongation ratio, and surface to volume ratio. The vesicles provided herein may be characterized by their ability to adjust the aggregates' shape and properties to the anisotropic stress caused by pore crossing. Sufficient adaptability implies that a vesicle or an aggregate can sustain different unidirectional forces or stress, such as one caused by pressure, without extensive fragmentation, which defines a “stable” aggregate. If an aggregate passes through a barrier fulfilling this condition the terms “adaptability” and (shape) “deformability” plus “permeability” are essentially equivalent. A “barrier” in the context of this disclosure is (as in, for example, EP 0 475 160 and WO 98/17255) a body with through-extending narrow pores, such narrow pores having a radius which is at least 25% smaller than the radius of the ESAs (considered as spherical) before said ESAs permeate through such pores.

The term “narrow” used in connection with a pore implies that the pore radius is significantly, typically at least 25%, smaller than the radius of the entity tested with regard to its ability to cross the pore. The necessary difference typically should be greater for the narrower pores. Using 25% limit is therefore quite suitable for >150 nm diameter whereas >100% difference requirement is more appropriate for the smaller systems, e.g., with <50 nm diameter. For diameters around 20 nm, aggregate diameter difference of at least 200% is often required.

The term “semipermeable” used in connection with a barrier implies that a solution can cross transbarrier openings whereas a suspension of non-adaptable aggregates (large enough for the above definition of “narrow” pores to apply) cannot. Conventional lipid vesicles (liposomes) made from any common phosphatidylcholine in the gel lamellar phase or else from any biological phosphatidylcholine/cholesterol 1/1 mol/mol mixture or else comparably large oil droplets, all having the specified relative diameter, are three examples for such non-adaptable aggregates.

The term “stable” means that the tested aggregates do not change their diameter spontaneously or under the transport related mechanical stress (e.g. during passage through a semipermeable barrier) unacceptably, which most often means only to a pharmaceutically acceptable degree. A 20-40% change is normally considered acceptable; the halving or doubling of aggregate diameter is borderline and a greater change in diameter is typically unacceptable. Alternatively, and very conveniently, the change in aggregate diameter resulting from pore crossing under pressure is used to assess system stability; the same criteria are then applied as for “narrow” pores, mutatis mutandis. To obtain the correct value for aggregate diameter change, a correction for flux/vortex effects may be necessary. These procedures are described in greater detail in the publications of the applicant in Cevc et. al., Biochim. Biophys. Acta 2002; 1564:21-30.

Non-destructing passage of ultradeformable, mixed lipid aggregates through narrow pores in a semi-permeable barrier is thus diagnostic of high aggregate adaptability. If pore radius is two times smaller than the average aggregate radius the aggregate must change its shape and surface-to-volume ratio at least 100% to pass without fragmentation through the barrier. An easy and reversible change in aggregate shape inevitably implies high aggregate deformability and requires large surface-to-volume ratio adaptation. A change in surface-to-volume ratio per se implies: a) high volume compressibility, e.g. in the case of compact droplets containing material other than, and immiscible with, the suspending fluid; b) high aggregate membrane permeability, e.g. in the case of vesicles that are free to exchange fluid between inner and outer vesicle volume.

4.8. Cell Viability and Cell Proliferation Assays

Many assays well-known in the art can be used to assess the proliferation and viability of bacterial cells or mycotic agents following exposure to the formulations provided herein. For example, cell proliferation can be assayed by measuring Bromodeoxyuridine (BrdU) incorporation, (3H) thymidine incorporation, by direct cell count, or by detecting changes in transcription, translation or activity of known genes such as proto-oncogenes fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies, mRNA can be quantitated using methods that are well known and routine in the art, for example, using northern analysis, RNase protection, or polymerase chain reaction in connection with reverse transcription.

Cell viability can be assessed by using trypan-blue staining or other cell death or viability markers known in the art. In a specific embodiment, the level of cellular ATP is measured to determined cell viability. In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic 40%), Ps (partially toxic-slight-20%), or 0 (no toxicity-0%), conforming to the degree of cytotoxicity seen. A 50% cell inhibitory (cytotoxic) concentration (IC₅₀) is determined by regression analysis of these data.

The toxicity and/or efficacy of a formulation in accordance with the invention can be determined by standard pharmaceutical procedures in cell cultures or experimental animals, e.g., for determining the LD₅₀ (the dose lethal to 50% or the population) and the ED₅₀ (the dose therapeutically effective in 50% of the population). The dose ratio between toxic and therapeutic effects is the therapeutic index and it can be expressed as the ratio LD₅₀/ED₅₀. A formulation identified in accordance with the invention that exhibits large therapeutic indices is preferred. While a formulation identified in accordance with the invention that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of a formulation identified in accordance with the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration or the test formulation that achieves a half-maximal inhibition of symptoms) as determined in cell culture. Such information can be used to more accurately determine useful doses in humans. Levels in plasma may be measured, for example, by high-performance liquid chromatography

4.9. Spore Count Assays

Any assay well known in the art can be used to determine the spore count of microbial agents following exposure to the formulations provided herein. For example, the viable microbial spore count can be measured by colony counting, and then the total microbial spore count can be measured by direct microscopic counting. The ratio of viable to total microbial spore count yields the fraction of spores that remain viable within a given sample.

A procedure for colony counting to determine endospore concentration is, for example, comprised of the steps of (1) heat shocking a microbial sample to kill vegetative cells while microbial spores remain viable, (2) plating a known volume of the sample with a known dilution factor onto a growth medium, and (3) incubating the growth plates for 2 days. Finally, the resulting visible colonies can be counted and reported as colony forming units (CFU's). A procedure for direct microscopic counting is, for example, comprised of the steps of (1) placing a microbial sample on a slide with an indentation of a known volume, and (2) counting the spores in each the several squares and multiplying the average count by an appropriate factor to yield the number of total cells per milliliter in the original suspension.

4.10. Methods of Administration

4.10.1 Plants

The formulations provided herein can be delivered to a plant in order to reduce the proliferation or viability of a microbial agent that has infected said plant.

Any species of woody, ornamental or decorative, crop or cereal, fruit or vegetable plant, and algae (e.g., Chlamydomonas reinhardtii) may be used in the methods provided herein. Non-limiting examples of plants include plants from the genus Arabidopsis or the genus Oryza. Other examples include plants from the genuses Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetium, Persea, Phaseolus, Physcomitrella, Picea, Pinus, Poncirus, Populus, Prunus, Robinia, Rosa, Saccharum, Schedunorus, Secale, Sesamum, Solanum, Sorghum, Stevia, Thellungiella, Theobroma, Triphysaria, Triticum, Vitis, Zea, or Zinnia.

In addition to a plant, the present invention provides any clone of such a plant, seed, selfed or hybrid progeny and descendants, and any part of any of these, such as cuttings, seed. The invention provides any plant propagule, that is any part which may be used in reproduction or propagation, sexual or asexual, including cuttings, seed and so on. Also encompassed by the invention is a plant which is a sexually or asexually propagated off-spring, clone or descendant of such a plant, or any part or propagule of said plant, off-spring, clone or descendant. Plant extracts and derivatives are also provided.

Plants included in the invention are any plants amenable to transformation techniques, including gymnosperms and angiosperms, both monocotyledons and dicotyledons.

Examples of monocotyledonous angiosperms include, but are not limited to, asparagus, field and sweet corn, barley, wheat, rice, sorghum, onion, pearl millet, rye and oats and other cereal grains.

Examples of dicotyledonous angiosperms include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals.

Examples of woody species include poplar, pine, sequoia, cedar, oak, etc.

Still other examples of plants include, but are not limited to, wheat, cauliflower, tomato, tobacco, corn, petunia, trees, etc.

In certain embodiments, plants of the present invention are crop plants (for example, cereals and pulses, maize, wheat, potatoes, tapioca, rice, sorghum, millet, cassaya, barley, pea, and other root, tuber, or seed crops. Exemplary cereal crops used in the compositions and methods of the invention include, but are not limited to, any species of crass, or grain plant (e.g., barley, corn, oats, rice, wild rice, rye, wheat, millet, sorghum, triticale, etc.), non-crass plants (e.g., buckwheat flax, legumes or soybeans, etc.). Grain plants that provide seeds of interest include oil-seed plants and leguminous plants. Other seeds of interest include grain seeds, such as corn, wheat, barley, rice, sorghum, rye, etc. Oil seed plants include cotton, soybean, safflower, sunflower, Brassica, maize, alfalfa, palm, coconut, etc. Other important seed crops are oil-seed rape, sugar beet, maize, sunflower, soybean, and sorghum. Leguminous plants include beans and peas. Beans include guar, locust bean, fenugreek, soybean, garden beans, cowpea, mungbean, lima bean, fava bean, lentils, chickpea, etc.

Horticultural plants to which the present invention may be applied may include lettuce, endive, and vegetable brassicas including cabbage, broccoli, and cauliflower, and carnations and geraniums. The present invention may also be applied to tobacco, cucurbits, carrot, strawberry, sunflower, tomato, pepper, chrysanthemum, poplar, eucalyptus, and pine.

The present invention may be used for transformation of other plant species, including, but not limited to, corn (Zea mays), canola (Brassica napus, Brussica rupa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum, Nicotiana benthamiana), potato (Solanum tuberosum), peanuts (Arachis hypogaea), cotton (Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, Arabidopsis spp., vegetables, ornamentals, and conifers.

4.10.1.1 Plant Transformation/Transfection Methods

Any method or delivery system may be used for the delivery and/or transfection of the formulations provided herein to plants. The formulations may be delivered to a plant either alone, or in combination with other agents.

Transfection may be accomplished by a wide variety of means, as is known to those of ordinary skill in the art. Such methods include, but are not limited to, Agrobacterium-mediated transformation (e.g., Komari et al., 1998, Curr. Opin. Plant Biol., 1:161), particle bombardment mediated transformation (e.g., Finer et al., 1999, Curr. Top. Microbiol. Immunol., 240:59), protoplast electroporation (e.g., Bates, 1999, Methods Mol. Biol., 111:359), viral infection (e.g., Porta and Lomonossoff, 1996, Mol. Biotechnol. 5:209), microinjection, and liposome injection. Other exemplary delivery systems that can be used to facilitate uptake by a cell of a formulation include calcium phosphate and other chemical mediators of intracellular transport, and microinjection compositions. Alternative methods may involve, for example, the use of electroporation, or chemicals that increase free (or “naked”) DNA uptake, transformation using viruses or pollen and the use of microprojection. Standard molecular biology techniques are common in the art (e.g., Sambrook et al., 1989, Molecular Cloning: A Laboratory Manual, 2nd ed., Cold Spring Harbor Laboratory Press, New York).

The transformation of plants in accordance with the invention may be carried out in essentially any of the various ways known to those skilled in the art of plant molecular biology. (See, for example, Methods of Enzymology, Vol. 153, 1987, Wu and Grossman, Eds., Academic Press, incorporated herein by reference).

Agrobacterium transformation is widely used by those skilled in the art to transform dicotyledonous species. Recently, there has been substantial progress towards the routine production of stable, fertile transgenic plants in almost all economically relevant monocot plants (Toriyarna et al., 1988, Bio/Technology 6:1072-1074; Zhang et al., 1988, Plant Cell Rep. 7:379-384; Zhang et al., 1988, Theor. Appl. Genet. 76:835-840; Shimamoto et al., 1989, Nature 338; 274-276; Datta et al., 1990, Bio/Technology 8: 736-740; Christou et al., 1991, Bio/Technology 9:957-962; Peng et al., 1991, International Rice Research Institute, Manila, Philippines, pp. 563-574; Cao et al., 1992, Plant Cell Rep, 11:585-591; Li et al., 1993, Plant Cell Rep. 12:250-255; Rathore et al., 1993, Plant Mol. Biol. 21:871-884; Fromm et al., 1990, Bio/Technology 8:833-839; Tomes et al., 1995, “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); D'Halluin et al., 1992, Plant Cell 4:1495-1505; Walters et al., 1992, Plant Mol. Biol. 18:189-200; Koziel et al., 1993, Biotechnology 11: 194-200; Vasil, I. K., 1994, Plant Mol. Biol. 25:925-937; Weeks et al., 1993, Plant Physiol. 102:1077-1084; Somers et al., 1992. Bio/Technology 10: 1589-1594; WO 92/14828). In particular, Agrobacterium mediated transformation is now emerging also as an highly efficient transformation method in monocots (filet et al., 1994. The Plant Journal 6:271-282), See also, Shimamoto, K., 1994, Current Opinion in Biotechnology 5:158-162; Vasil et al. 1992. Bio/Technology 10.667-674; Vain et al., 1995, Biotechnology Advances 13(4):653-671; Vasil et al., 1996, Nature Biotechnology 14:702).

The particular choice of a transformation technology will be determined by its efficiency to transform certain plant species as well as the experience and preference of the person practicing the invention with a particular methodology of choice. It will be apparent to the skilled person that the particular choice of a transformation system to introduce the formulations provided herein into plant cells is not essential to or a limitation of the invention, nor is the choice of technique for plant regeneration.

4.10.2 Human and Animal Subjects

The formulations provided herein can be delivered to an animal in order to reduce the proliferation or viability of a microbial agent that has infected said animal. Any animal can be used in the methods described herein, including but not limited to, birds, reptiles, and mammals, such as a mammal including a non-primate (e.g., a camel, donkey, zebra, cow, pig, horse, goat, sheep, cat, dog, rat, and mouse) and a primate (e.g., a monkey, chimpanzee, undo human). In a specific embodiment, the animal is a human.

Provided herein are methods of administering a pharmaceutical composition comprising an antimicrobial provided herein, a lipid, and a surfactant. The formulations may be administered topically, including mucosal delivery. Mucosal delivery includes pulmonary, oropharyngeal, genitourinary, ocular, and nasal delivery.

Pulmonary administration can be employed, e.g., by use of an inhaler, or nebulizer, and formulation with an aerosolizing agent, or via perfusion in a fluorocarbon or synthetic pulmonary surfactant. In certain embodiments, the formulations provided herein can be formulated as a suppository, with traditional binders and carriers such as triglycerides.

In one embodiment, the formulations provided therein are lyophilized to allow for pulmonary delivery. In one embodiment, the formulations provided herein are lyophilized by mixing the formulation with a diluent to form a liquid composition and then lyophilizing the liquid composition to form a lyophilate. The formulations may be lyophilized by any method known in the art for lyophilizing a liquid.

A formulation is preferably administered as a component of a composition that optionally comprises a pharmaceutically acceptable carrier, excipient or diluent.

4.10.2.1 Dosage and Frequency of Administration

The amount of a formulation that will be effective in inhibiting the proliferation or viability of a microbial agent that has infected a human or animal can be determined by standard clinical techniques. In vitro or in vivo assays may optionally be employed to help identify optimal dosage ranges. The precise dose to be employed will also depend, e.g., on the route of administration, the type of microbial infection, type of microbial disease, and the seriousness of the microbial infection, and should be decided according to the judgment of the practitioner and each patient's or subject's circumstances.

In some embodiments, the formulations provided herein comprise from about 1 to about 20 mg of the antimicrobial. For instance, the formulations can comprise about 1, about 2, about 3, about 4, about 5, about 6, about 7, about 8, about 9, about 10, about 11, about 12, about 13, about 14, about 15, about 16, about 17, about 18, about 19, or about 20 mg of the antimicrobial.

In some embodiments, the formulations provided herein comprise from about 1 to about 500 μg of the antimicrobial. For instance, the formulations can comprise about 1, about 25, about 50, about 75, about 100, about 125, about 150, about 175, about 200, about 225, about 250, about 275, about 300, about 325, about 350, about 375, about 400, about 425, about 450, about 475, or about 500 μg of the antimicrobial.

Exemplary doses of a formulation include milligram (mg) or microgram (μg) amounts per kilogram (Kg) of subject or sample weight per day (e.g., from about 1 μg per Kg to about 500 mg per Kg per day, from about 5 μg per Kg to about 100 mg per Kg per day, or from about 10 μg per Kg to about 100 mg per Kg per day. In specific embodiments, a daily dose is at least 0.1 mg, 0.5 mg, 1.0 mg, 2.0 mg, 5.0 mg, 10 mg, 25 mg, 50 mg, 75 mg, 100 mg, 150 mg, 250 mg, 500 mg, 750 mg, or at least 1 g. In another embodiment, the dosage is a unit dose of about 0.1 mg, 1 mg, 5 mg, 10 mg, 50 mg, 100 mg, 150 mg, 200 mg, 250 mg, 300 mg, 350 mg, 400 mg, 500 mg, 550 mg, 600 mg, 650 mg, 700 mg, 750 mg, 800 mg or more. In another embodiment, the dosage is a unit dose that ranges from about 0.1 mg to about 1000 mg, 1 mg to about 1000 mg, 5 mg to about 1000 mg, about 10 mg to about 500 mg, about 150 mg to about 500 mg, about 150 mg to about 1000 mg, 250 mg to about 1000 mg, about 300 mg to about 1000 mg, or about 500 mg to about 1000 mg. In one embodiment, a subject is administered one or more doses of an effective amount of a formulation or a pharmaceutical composition thereof, wherein the effective amount is not the same for each dose.

Standard antimicrobial regimens have often been largely designed to administer the highest dose of antimicrobial agent without undue toxicity, i.e., often referred to as the “maximum tolerated dose” (MID) or “no observed adverse effect level” (NOAEL). In specific embodiments, one or more antimicrobial formulations are delivered to a subject (preferably, a human subject) at a dosage lower than the MTD of an unformulated antimicrobial agent or the no observed adverse effect level NOAEL of an unformulated antimicrobial agent. In specific embodiments, one or more antimicrobial formulations are delivered to a subject (preferably, a human subject) at a dosage lower than the human equivalent dose (“HED”) of the NOAEL of an unformulated antimicrobial agent. In certain embodiments, one or more antimicrobial formulations are delivered to a subject in need thereof at a 5% to 40%, preferably a 25% to 75% and more preferably a 25% to 99% lower dosage than the MTD of an unformulated antimicrobial agent or the NOAEL of the unformulated antimicrobial agent. In certain embodiments, one or more antimicrobial formulations are delivered to a subject in need thereof at a 5% to 40%, preferably a 25% to 75% and more preferably a 25% to 99% lower dosage than the MTD of an unformulated antimicrobial agent or HED of the NOAEL of an unformulated antimicrobial agent.

The MTDs of most of the antimicrobial agents described herein are well-known and are typically based on the results of Phase I dose escalation trials. In specific embodiments, the dose used for an antimicrobial formulation of the invention is at least 10%, 15%, 20%, 30%, 40%, 50%, 60%, 70%, 80% or 90% less than the MTD of an unformulated antimicrobial agent. In other specific embodiments, the dose used for and antimicrobial formulation of the invention is at least 1.5-, 1.8-, 2-, 3-, 4-, 5-, 10-, 25-, or 100-fold less than the MTD of an unformulated antimicrobial agent.

In specific embodiments, the dose used for an antimicrobial formulation of the invention is at least 10%, 15%, 20%, 30%, 40%, 50%, 60%, 70%, 80% or 90% less than the NOAEL for of an unformulated antimicrobial agent. In other specific embodiments, the dose used for and antimicrobial formulation of the invention is at least 1.5-, 1.8-, 2-, 3-, 4-, 5-, 10-, 25-, or 100-fold less than the NOAEL of an unformulated antimicrobial agent.

The NOAEL, as determined in animal studies, is often used determining the maximum recommended starting dose for human clinical trials. The NOAELs can be extrapolated to determine human equivalent dosages (HEDs). Typically, such extrapolations between species are conducted based on the doses that are normalized to body surface area (i.e., mg/m²). In specific embodiments, the NOAELs are determined in either mice, hamsters, rats, ferrets, guinea pigs, rabbits, dogs, primates, primates (monkeys, marmosets, squirrel monkeys, baboons), micropigs and minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. Accordingly, in certain embodiments, the regimen comprises administering a therapy at a dose less than the HED. For instance, the invention provides a method of preventing recurrence of cancer in a subject in remission, the method comprising administering to a subject in need thereof a prophylactically effective regimen, the regimen comprising administering one or more therapies to the subject at dose less than the HED.

In certain embodiments of the methods, the administration of formulations provided herein results in a mean serum concentration of the antimicrobial in the human subject of less than 10 ng/mL, 5 ng/mL, 4 ng/mL, 3 ng/mL, 2 ng/mL, 1 ng/mL, 0.5 ng/mL, or 0.2 ng/mL. In some embodiments of the methods, the formulation comprises about 1 to about 5 mg of an antimicrobial provided herein. In a specific embodiment of the method, the pharmaceutical composition comprises 3 mg of an antimicrobial provided herein.

In one embodiment, the formulations described herein are administered in multiple doses. When administered in multiple doses, the formulations are administered with a frequency and in an amount sufficient to treat the condition. In one embodiment, the frequency of administration ranges from once a day up to about once every eight weeks. For example, the formulations can be administered once a week, once every two weeks, once every three weeks or once every four weeks. In another embodiment, the frequency of administration ranges from about once a week up to about once every six weeks. In another embodiment, the frequency of administration ranges from about once every two weeks up to about once every four weeks. In certain embodiments, the daily, weekly, or multi-weekly administration may be continued for several cycles as determined by the physician and the nature of the cancer. In certain embodiments, the number of cycles may be about 1, 2, 5, 8, 10, 15, 20, 25 or 30.

The formulation can be administered, for example, once or twice daily. In certain embodiments, the composition may also be administered, once every two days, once daily, three times a day or four times a day. In certain embodiments, the formulation is administered for at least three weeks. In other embodiments, the formulation is administered for 1 to 48 weeks, 1 to 36 weeks or 1 to 24 weeks, 1 to 12 weeks or 1 to 6 weeks.

In certain embodiments of the methods, the formulation may also be administered, once every two days, daily, three times a day or four times a day. In specific embodiments, the formulation is administered for 1 to 48 weeks, 1 to 36 weeks, 1 to 24 weeks, 1 to weeks or 1 to 6 weeks.

In one embodiment, the fungal infection being treated is not onchymycosis.

In some embodiments of the methods described herein, topical formulation is administered for a period longer than 12 weeks. For instance, in some embodiments, the formulation is administered for at least 24 weeks, for at least 36 weeks, or for at least 48 weeks.

In some embodiments of the methods, a cyclical treatment regimen is employed. Such regimens employ treatment cycles involving the administration of the formulation for a period of time, followed by a period wherein no formulation is administered, and, if necessary, repeating this sequence, i.e., the cycle. Treatment cycles can include, for example, administering, the topical formulation consecutively for a period up to 48 weeks (e.g., 12 weeks), e.g., using once or twice daily administration, followed a period of time wherein no formulation is administered, followed by another period where the formulation is again administered consecutively for another 12 weeks.

4.12 Screening Assays

Provided herein are screening assays to identify compounds that can inhibit the proliferation, viability, or sporulation of a microbial agent. The test compounds used in the screening methods provided herein include any compound that can inhibit the proliferation, viability, or sporulation of a microbial agent, including a mycotic agent, a bacterial agent, or a mycoplasma.

In particular, the present invention includes methods of screening compounds for antifungal activity comprising contacting a mycotic agent with an effective amount of a compound, wherein said compound is formulated with a lipid and a surfactant, and detecting a reduction in the proliferation, viability or sporulation of said mycotic agent, wherein said compound is adsorbed by the phospholipid membranes of the Spitzenkorper or Polarsiome regions of the hypha of said mycotic agent. The present invention also includes methods of screening compounds for antibacterial activity comprising contacting a bacterial agent with an effective amount of a compound, wherein said compound is formulated with a lipid and a surfactant, and detecting a reduction in the proliferation, viability or sporulation of said bacterial agent, wherein said compound is adsorbed by the phospholipid membranes of the bacterial agent. The present invention also includes methods of screening compounds for antimycoplasma activity comprising contacting a mycoplasma with an effective amount of a compound, wherein said compound is formulated with a lipid and a surfactant, and detecting a reduction in the proliferation, viability or sporulation of said bacterial agent, wherein said compound is adsorbed by the phospholipid membranes of the mycoplasma.

4.13 Kits

The disclosure further includes a pharmaceutical pack or kit comprising one or more containers filled with a formulation provided herein for the treatment or prevention of a fungal or bacterial infection in a human subject. The disclosure provides kits that can be used in the above-described methods.

In one embodiment, a kit comprises one or more containers comprising an antimicrobial formulation provided herein. The kit may further comprise instructions for administering the antimicrobial formulations provided herein for the treating or preventing skin and/or nail infections, as well as side effects and dosage information. Optionally associated with such container(s) can be a notice in the form prescribed by a governmental agency regulating the manufacture, use or sale for human administration.

### 6. EXAMPLES

### 6.1 Example 1

### Evaluation of the Morphological Effects of Antifungal Preparations on Dermatophyte Hyphae In Vitro

The morphological changes to dermatophyte hyphae following exposure to an antifungal formulation of the invention, specifically a terbinafine formulation, compared to terbinafine hydrochloride solution in vitro were evaluated.

Trichophyton rubrum MYA4498, one of the quality control isolates approved by the Clinical and Laboratory Standards Institute (CLSI) for dermatophyte susceptibility testing, was used as a test isolate throughout testing, (See, Ghannoum et al., 2004, J Clin Microbiol. 42(7): 2977-2979; Ghannoum et al., J Clin Microbiol. 44: 353-4350. Inoculum containing 3×10³ conidia/ml of T. rubrum was prepared in RPMI-1640 buffered with MOPS (Hardy Diagnostics, Santa Maria. CA), added to the wells of microtiter plates (100 ul aliquots) and incubated at 35° C. for 2-3 days until good hyphal growth was achieved. Specific concentrations of 1 mg/ml, 3 mg/ml, and 15 mg/ml of terbinafine alone were prepared in RPMI-1640. The terbinafine hydrochloride (1 mg/ml. 3 mg/ml, and 15 mg/ml) and 15 mg/ml of a terbinafine formulation of the invention was added to the wells of the microtiter plates (100 ul aliquots) and re-incubated. Twenty plates for each drug were set up to ensure adequate sample for examination over several weeks; RPMI was added as needed to maintain the volume of each well at 200 ul.

At pre-determined intervals (24, 48, 72, and 96 hours and once weekly thereafter for a total of 12 weeks), a loopful of hyphal growth from the bottom of each well was removed to a glass microscope slide containing a drop of calcofluor white stain (fluorescent KOH) and covered with a cover slip. Slides were examined microscopically under both white light and UV light. Images from a representative field visualized under each light source were be recorded.

Samples showing significant differences in morphology between exposure of a terbinafine formulation vs. terbinafine alone were further examined under Scanning Electron Microscopy (SEM). Preparation of samples for SEM were performed following previously established methodology (see. Chandra et al., 2001. J. Dent. Res. 80:903-908).

6.1.1. Results

As indicated by Tables 9 and 10, for 24, 48, 72 and 96 hours there were no morphological changes observed in the samples incubated with the growth control, 1 μg/mL of terbinafine hydrochloride, or 3 mg/mL of terbinafine hydrochloride. There were morphological changes observed at every time point (24, 48, 72, and 96 hours) for the hyphae exposed to a terbinafine formulation of the invention. In particular, a faster occurrence of vacuole formation was observed in the samples incubated with the terbinafine formulation as opposed to terbinafine hydrochloride, which is indicative of intracellular destruction.

As indicated by Table 11, after one week of drug exposure, terbinafine 3 mg/ml, and terbinafine 15 mg/ml showed no presence of vacuoles within the hyphae, while terbinafine 1 μg/ml and the terbinafine formulation both had vacuoles within the hyphae. At two weeks all of the tested drugs and concentrations had the appearance of vacuoles within the hyphae.

TABLE 9

| | Time | |
|---|---|---|
| | 24 hour Change | 48 hour Change |
| | in morphology | in morphology |
| | If yes, descrip- | If yes, descrip- |
| | tion of change | tion of change |
| Growth Control | no | no |
| Terbinafine 1 μg/mL | no | no |
| Terbinafine 3 mg/mL | no | no |
| Terbinafine 15 mg/mL | no | no |
| Terbinafine formulation | Yes: hyphae under | Yes: hyphae under |
| | white light appear | white light appear |
| | to have vacuoles | to have vacuoles |

TABLE 10

| | Time | |
|---|---|---|
| | 72 hour Change | 96 hour Change |
| | in morphology | in morphology |
| | If yes, descrip- | If yes, descrip- |
| | tion of change | tion of change |
| Growth Control | no | no |
| Terbinafine 1 μg/mL | no | no |
| Terbinafine 3 mg/mL | no | no |
| Terbinafine 15 mg/mL | no | no |
| Terbinafine formulation | Yes: hyphae under | Yes: hyphae under |
| | white light appear | white light appear |
| | to have vacuoles | to have vacuoles |

TABLE 11

| | Time | |
|---|---|---|
| | Week 1 Change | Week 2 Change |
| | in morphology | in morphology |
| | If yes, descrip- | If yes, descrip- |
| | tion of change | tion of change |
| Growth Control | no | no |
| Terbinafine 1 μg/mL | Yes: hyphae under | Yes: hyphae under |
| | white light appear | white light appear |
| | to have vacuoles | to have vacuoles |
| Tcrbinafine 3 mg/mL | no | Yes: hyphae under |
| | | white light appear |
| | | to have vacuoles |
| Terbinafine 15 mg/mL | no | no |
| Terbinafine formulation | Yes: hyphae under | Yes: hyphae under |
| | white light appear | white light appear |
| | to have vacuoles | to have vacuoles |

### 6.2 Example 2

### Determination of Minimum Inhibitory and Fungicidal Concentration

Antifungal activity of the antifungal formulations of the invention against dermatophytes, as compared to terbinafine hydrochloride alone, is determined in various dermatophytes known to cause onychomycosis, including Trichophyton rubrum, T. mentagrophytes, and Epidermophyton floccosum. Antifungal activity of the antifungal formulations of the invention as compared to terbinafine hydrochloride alone, was determined in various pathogenic fungi, including Aspergillus flavus and Aspergillus fumigatus. Antifungal activity of the antifungal formulations of the invention was measured by the minimum inhibitory concentration (MIC). Antifungal activity can also be measured by minimum fungicidal concentration (MFC).

Several strains of Aspergillus flavus, Aspergillus fumigatus, and Candida albicans were tested. Trichophyton rubrum MYA4498 and T. mentagrophytes MYA4439, the QC isolates approved by the Clinical and Laboratory Standards Institute (CLSI) for dermatophyte susceptibility testing, can also be tested.

MIC testing was performed according to the CLSI M38A2 standard for the susceptibility testing of dermatophytes developed at the Center for Medical Mycology (See, Ghannoum et al., 2004, J Clin Microbial. 42(7): 2977-2979 CLSI, Reference Method for Broth Dilution Antifungal Susceptibility Testing of Filamentous Fungi: Approved Standard-Second Edition. CLSI document M38-A2 [ISBN 1-56238-668-9]. CLSI, 940 West Valley Road, Suite 1400, Wayne, Pa. 19087-1898 USA, 2008). Briefly, RPMI was the test medium, incubation temperature and time was 35° C. and 24 hours or 48 hours, respectively, and the inoculum size was 1-3×10³ conidia/ml. The MIC endpoint was 100% inhibition as compared to the growth control.

MFC determinations are performed according to the modifications previously described (Canton et al., 2003. Diagn Microbiol Infect Dis. 45:203-6; Ghannoum and Isham, 2007. Infectious Diseases in Clinical Practice. 15(4):250-253). Specifically, the total contents of each clear well from the MIC assay can be subcultured onto potato dextrose agar. To avoid antifungal carryover, the aliquots are allowed to soak into the agar and then were streaked for isolation once dry, thus removing the cells from the drug source. Fungicidal activity is defined as a ≧99.9% reduction in the number of colony forming units (CFU)/ml from the starting inoculum count. Fungistatic activity is defined as <99.9% reduction.

The MIC range, MIC₁₀₀ (defined as the minimum concentration required to inhibit 100% of the isolates tested), for the antifungal preparations of the invention and comparators was computed. The MFC range, MFC₅₀, MFC₉₀, and MFC₁₀₀ for the antifungal preparations of the invention and comparators can also be computed.

The lowest concentration (μg/mL) of a terbinafine formulation of the invention as compared to terbinafine hydrochloride alone that is required to inhibit 100% growth of various isolates of Aspergillus flavus and Aspergillus fumigatus (MIC₁₀₀) in 24 hours and 48 hours was determined (see Table 12 and Table 13). In this example, the terbinafine formulation of the invention comprises terbinafine formulated with a phospholipid and a surfactant.

TABLE 12
MIC₁₀₀ of Terbinafine Formluation and
Terbinafine Hydrochloride in 24 hours

| | | Terbinafine | |
|---|---|---|---|
| | | formulation of | Terbinafine |
| | | the invention | hydrochloride |
| Strain | | MIC₁₀₀ (μg/mL) | MIC₁₀₀ (μg/mL) |
| MRL No. | Organism | in 24 hours | in 24 hours |
| 17840 | A. flavus | 0.00003 | 0.002 |
| 17842 | A. flavus | 0.002 | 0.002 |
| 18736 | A. flavus | 0.008 | 0.00006 |
| 18744 | A. flavus | 0.015 | 0.008 |
| 18743 | A. flavus | 0.015 | 0.008 |
| 2456 | A. fumigatus | 0.0012 | 0.25 |
| 2471 | A. fumigatus | 0.06 | 0.25 |
| 2574 | A. fumigatus | 0.06 | 0.5 |
| 16621 | A. fumigatus | 0.25 | 0.5 |
| 16629 | A. fumigatus | 0.25 | 0.5 |

TABLE 13
MIC₁₀₀ of Terbinafine Formluation and
Terbinafine Hydrochloride in 48 hours

| | | Terbinafine | |
|---|---|---|---|
| | | formulation of | Terbinafine |
| | | the invention | hydrochloride |
| Strain | | MIC₁₀₀ (μg/mL) | MIC₁₀₀ (μg/mL) |
| MRL No. | Organism | in 48 hours | in 48 hours |
| 17840 | A. flavus | 0.002 | 0.004 |
| 17842 | A. flavus | 0.015 | 0.002 |
| 18736 | A. flavus | 0.015 | 0.002 |
| 18744 | A. flavus | 0.03 | 0.002 |
| 18743 | A. flavus | 0.03 | 0.015 |
| 2456 | A. fumigatus | 0.5 | 234 |
| 2471 | A. fumigatus | 1 | 234 |
| 2574 | A. fumigatus | 1 | 234 |
| 16627 | A. fumigatus | 15 | 234 |
| 16629 | A. fumigatus | 15 | 234 |

As depicted in Tables 12 and 13, a lower concentration of terbinafine is generally able to inhibit 100% growth of various isolates of Aspergillus flavus and Aspergillus fumigatus at 24 and 48 hours when terbinafine is in formulation than the concentration required when terbinafine is not in formulation. These results indicate that the efficacy of action of terbinafine is significantly enhanced by formulation of terbinafine with a phospholipid and a surfactant.

Similarly, the lowest concentration (μg/mL) of a fluconazole formulation of the invention was compared to flucanozole alone that is required to inhibit 100% growth of various isolates of Candida albicans (MIC₁₀₀) was determined (see Table 14). In this example, the fluconazole formulation of the invention comprises fluconazole formulated with a phosopholipid and a surfactant.

TABLE 14
MIC₁₀₀ of Flucanozole Formulation and Flucanozole

| | | Flucanozole | |
|---|---|---|---|
| | | formulation of | |
| Strain | | the invention | Flucanozole |
| MRL No. | Organism | MIC₁₀₀ (μg/mL) | MIC₁₀₀ (μg/mL) |
| 648 | C. albicans | 0.6 | >64 |
| 9650 | C. albicans | 0.3 | >128 |
| 9698 | C. albicans | 1.25 | 32 |
| 14461 | C. albicans | 0.08 | 0.25 |
| 14465 | C. albicans | 0.16 | 0.25 |

As depicted in Table 14, a lower concentration of fluconazole is generally able to inhibit 100% growth of various isolates of Candida albicans when fluconazole is in formulation than the concentration required when fluconazole is not in formulation. These results indicate that the efficacy of action of fluconazole is significantly enhanced by formulating fluconazole with a phospholipid and a surfactant.

The lowest concentration (μg/mL) of either a terbinafine formulation of the invention or voriconazole, when used in combination, that is required to inhibit 100% of various isolates of Aspergillus flavus and Aspergillus fumigatus (MIC₁₀₀) was determined (see Table 15). In this example, the terbinafine formulation of the invention comprises terbinafine formulated with a phospholipid and a surfactant.

TABLE 15
MIC₁₀₀ of Viroconazol, Terbinafine Formulation, and
Combination of Voriconazole and Terbinafine Formulation

| Strain | | | | | Terbinafine | |
|---|---|---|---|---|---|---|
| MRL | | Voriconazole | Terbinafine | Voriconazole | formulation | |
| NO. | Organism | Individual | formulation | Combination | Combination | FICI |
| 2456 | A. fumigatus | 0.12 | 0.5 | 0.06 | 0.12 | 0.74 |
| 2471 | A. fumigatus | 0.25 | 05 | 0.06 | 0.12 | 0.48 |
| 2574 | A. fumigatus | 0.25 | 0.5 | 0.06 | 0.12 | 0.48 |
| 16627 | A. fumigatus | 0.5 | 16 | 0.03 | 4 | 0.31 |
| 16629 | A. fumigatus | 0.12 | 1 | 0.03 | 0.5 | 0.75 |
| 17842 | A. flavus | 0.25 | 0.016 | 0.12 | 0.001 | 0.54 |
| 18736 | A. flavus | 0.5 | 0.016 | 0.12 | 0.004 | 0.49 |
| 18743 | A. flavus | 0.5 | 0.03 | 0.12 | 0.016 | 0.77 |
| 18744 | A. flavus | 0.8 | 0.03 | 0.03 | 0.016 | 0.59 |

Depicted in Table 15, the Fractional Inhibitory Coefficient Index (FWD measures the degree of interaction between the two antifungal agents. A FICI value of greater than 4 indicates an antagonistic interaction; a FICI value of between 0.5 and 4 indicates no interaction; and a FICI value of less than 0.5 indicates a synergistic interaction. As depicted in Table 15, a lower concentration of either voriconazole or terbinafine formulation is generally able to inhibit 100% growth of various isolates of Aspergillus fumigatus and Aspergillus flavus when both antifungal agents are used in combination than the concentration required to inhibit 100% growth of various isolates of Aspergillus fumigatus and Aspergillus flavus when either voriconazole or terbinafine formulation is used individually. That is, terbinafine formulation in combination with voriconazole exhibited a synergistic effect in three out of five A. fumigatus strains and one out of four A. flavus strains, while exhibiting no antagonistic interactions. These results indicate that a combination of one or more antifungal agents elicit their effects synergistically to reduce the proliferation or viability of a mycotic agent.

### 6.3 Example 3

### Antimicrobial Formulations

Antimicrobial formulations for topical application may be prepared by the following procedure:

1. Organic Phase Production, which Contains all Lipophilic Excipients

The organic phase is produced by weighing the lipid, the surfactant, an antimicrobial, and any additional lipophilic excipients into suitable containers followed by mixing these components into an optically isotropic phase which appears as a clear solution, wherein the antimicrobial is an antifungal selected from the group consisting of itraconazole, ketoconazole, posaconazole, saperconazole, SCH-50002, terconazole, butenafine, and griscofulvin; and hydrates, solvates, and salts thereof. During mixing, the organic phase will be heated up to a temperature of about 5 to about 60° C.

### 2. Aqueous Phase Production

The aqueous phase is prepared by weighing the non-lipophilic components and water, which serves as solvent, into suitable containers and then mixing these components into a clear solution. During mixing, the temperature will be elevated to about 5 to about 60° C.

### 3. Production of a Concentrated Intermediate by Combination of Both Phases

The isotropic organic phase and the clear aqueous phase are combined under stirring in a suitable vessel. Before and during the combination the temperature of both phases must be kept between about 5 to about 60° C. or between about 35 and about 45° C. The resulting intermediate is homogenised mechanically at a temperature of about 5 to about 60° C., e.g., about 40° C. Before starting homogenisation, the pressure in the production vessel is lowered to −0.08 MPa. The desired average carrier size is typically reached after 10 minutes of homogenisation.

Three process parameters must be controlled carefully during the production of the concentrated intermediate: temperature, homogeniser circulation velocity, and overall processing time.

4. Production of the Final Bulk Product by Mixing the Concentrated Intermediate with Dilution Buffer.

The concentrated intermediate is diluted with the dilution buffer to the intended final concentration. The mixture is carefully stirred in the mixing vessel at 20° C. to homogeneity.

Table 16 describes the amount of surfactant, lipid, and the antimicrobial in some antifungal formulations provided herein. The amount of the antimicrobial, lipid, lipid, and surfactant combined is described in terms of the percent total in the formulation.

TABLE 16
Examples of Antimicrobial Formulations
Table 16A: This table lists the relative amounts of each of the components of Exemplary Antimicrobial Formulations

| | | Lipid | | | | | | | |
|---|---|---|---|---|---|---|---|---|---|
| | | mg/g | Surfactant | | | | | | |
| | Antimicrobial | (1 to 10% | mg/g | Buffer | Antimicrobials | Antioxidants | Emollient | Other | Chelator |
| | (1-50 mg/g) | by wt.) | (1 to 10% by wt.) | (pH 4-7.5) | (0-10 mg/g) | (0-10 mg/g) | (0-50 mg/g) | (0-50 mg/g) | (0-25 mg/g) |
| 1 | 10 | 47.944 | 42.056 | 4 | 5.250 | 0.700 | 30.000 | 30.000 | 3.000 |
| 2 | 15 | 53.750 | 31.250 | 4 | 5.250 | 0.700 | 30.000 | 15.000 | 3.000 |
| 3 | 30 | 90.561 | 79.439 | 4 | 5.250 | 0.700 | 30.000 | 30.000 | 3.000 |
| 4 | 10 | 47.944 | 42.056 | 5 | 5.250 | 0.700 | 30.000 | 30.000 | 3.000 |
| 5 | 5 | 50.607 | 44.393 | 5 | 5.250 | 0.700 | 0.000 | 10.000 | 3.000 |
| 6 | 30 | 90.561 | 79.439 | 5 | 5.250 | 0.700 | 30.000 | 30.000 | 3.000 |
| 7 | 7.5 | 49.276 | 43.224 | 6.5 | 5.250 | 0.700 | 30.000 | 30.000 | 3.000 |
| 8 | 15 | 53.750 | 31.250 | 6.5 | 5.250 | 0.200 | 30.000 | 0.000 | 3.000 |
| 9 | 30 | 90.561 | 79.439 | 6.5 | 5.250 | 0.200 | 30.000 | 20.000 | 3.000 |
| 10 | 10 | 41.351 | 48.649 | 4 | 5.250 | 0.200 | 30.000 | 30.000 | 3.000 |
| 11 | 15 | 47.882 | 37.118 | 4 | 5.250 | 0.200 | 0.000 | 30.000 | 3.000 |
| 12 | 30 | 95.764 | 74.236 | 4 | 5.250 | 0.200 | 30.000 | 30.000 | 3.000 |
| 13 | 10 | 65.676 | 24.324 | 5 | 5.250 | 0.200 | 0.000 | 25.000 | 3.000 |
| 14 | 15 | 62.027 | 22.973 | 5 | 5.250 | 0.200 | 0.000 | 30.000 | 3.000 |
| 15 | 30 | 124.054 | 45.946 | 5 | 5.250 | 0.200 | 15.000 | 30.000 | 3.000 |
| 16 | 5 | 62.687 | 32.313 | 6.5 | 5.250 | 0.200 | 15.000 | 0.000 | 3.000 |
| 17 | 15 | 41.853 | 43.147 | 6.5 | 5.250 | 0.200 | 30.000 | 30.000 | 3.000 |
| 18 | 30 | 95.764 | 74.236 | 6.5 | 5.250 | 0.200 | 0.000 | 30.000 | 3.000 |
| 19 | 15 | 47.882 | 37.118 | 6.5 | 5.250 | 0.200 | 0.000 | 0.000 | 3.000 |
| 20 | 10 | 45.000 | 45.000 | 6.5 | 5.250 | 0.200 | 0.000 | 0.000 | 3.000 |
| 21 | 10 | 31.935 | 58.065 | 5 | 5.250 | 0.200 | 30.000 | 15.000 | 3.000 |
| 22 | 15 | 42.500 | 42.500 | 6.5 | 5.250 | 0.200 | 30.000 | 0.000 | 3.000 |
| 23 | 10 | 38.276 | 51.724 | 4 | 5.250 | 0.200 | 0.000 | 30.000 | 3.000 |
| 24 | 15 | 42.500 | 42.500 | 4 | 5.250 | 0.200 | 0.000 | 15.000 | 3.000 |
| 25 | 30 | 85.000 | 85.000 | 4 | 5.250 | 0.200 | 30.000 | 30.000 | 3.000 |
| 26 | 10 | 38.276 | 51.724 | 5 | 5.250 | 0.200 | 30.000 | 0.000 | 3.000 |
| 27 | 15 | 36.429 | 48.571 | 5 | 5.250 | 0.200 | 30.000 | 30.000 | 3.000 |
| 28 | 30 | 72.299 | 97.701 | 5 | 5.250 | 0.200 | 30.000 | 15.000 | 3.000 |
| 29 | 7.5 | 46.250 | 46.250 | 6.5 | 5.250 | 0.700 | 0.000 | 20.000 | 3.000 |
| 30 | 15 | 38.804 | 46.196 | 6.5 | 5.250 | 0.700 | 15.000 | 30.000 | 3.000 |
| 31 | 30 | 36.667 | 33.333 | 6.5 | 5.250 | 0.700 | 30.000 | 10.000 | 3.000 |
| 32 | 10 | 66.667 | 23.333 | 4 | 5.250 | 0.200 | 0.000 | 0.000 | 3.000 |
| 33 | 12.5 | 45.833 | 41.667 | 4 | 5.250 | 0.200 | 30.000 | 0.000 | 3.000 |
| 34 | 30 | 31.957 | 38.043 | 4 | 5.250 | 0.200 | 0.000 | 30.000 | 3.000 |
| 35 | 10 | 47.143 | 42.857 | 5 | 5.250 | 0.200 | 30.000 | 25.000 | 3.000 |
| 36 | 15 | 96.905 | 88.095 | 5 | 5.250 | 0.200 | 30.000 | 20.000 | 3.000 |
| 37 | 30 | 31.957 | 38.043 | 5 | 5.250 | 0.200 | 0.000 | 30.000 | 3.000 |
| 38 | 10 | 35.455 | 54.545 | 6.5 | 5.250 | 0.700 | 30.000 | 0.000 | 3.000 |
| 39 | 15 | 84.457 | 100.543 | 6.5 | 5.250 | 0.700 | 30.000 | 30.000 | 3.000 |
| 40 | 30 | 89.048 | 80.952 | 6.5 | 5.250 | 0.700 | 30.000 | 30.000 | 3.000 |
| 41 | 10 | 41.087 | 48.913 | 4 | 5.250 | 0.700 | 30.000 | 30.000 | 3.000 |
| 42 | 15 | 45.280 | 39.720 | 4 | 5.250 | 0.700 | 0.000 | 0.000 | 3.000 |
| 43 | 30 | 107.500 | 62.500 | 4 | 5.250 | 0.700 | 30.000 | 30.000 | 3.000 |
| 44 | 5 | 77.243 | 67.757 | 4 | 5.250 | 0.700 | 0.000 | 15.000 | 3.000 |
| 45 | 15 | 45.280 | 39.720 | 5 | 5.250 | 0.700 | 0.000 | 20.000 | 3.000 |
| 46 | 30 | 90.561 | 79.439 | 5 | 5.250 | 0.700 | 0.000 | 30.000 | 3.000 |
| 47 | 10 | 47.944 | 42.056 | 5 | 5.250 | 0.700 | 0.000 | 10.000 | 3.000 |
| 48 | 5 | 50.607 | 44.393 | 5.5 | 5.250 | 0.700 | 30.000 | 0.000 | 3.000 |
| 49 | 30 | 107.500 | 62.500 | 5.5 | 5.250 | 0.700 | 30.000 | 0.000 | 3.000 |
| 50 | 10 | 47.944 | 42.056 | 5.5 | 5.250 | 0.700 | 30.000 | 30.000 | 3.000 |
| 51 | 15 | 46.364 | 38.636 | 4 | 5.250 | 0.200 | 30.000 | 25.000 | 3.000 |
| 52 | 15 | 46.364 | 38.636 | 4 | 5.250 | 0.200 | 0.000 | 20.000 | 3.000 |
| 53 | 10 | 46.098 | 43.902 | 5 | 5.250 | 0.200 | 15.000 | 30.000 | 3.000 |
| 54 | 15 | 43.537 | 41.463 | 5 | 5.250 | 0.200 | 30.000 | 0.000 | 3.000 |
| 55 | 10 | 45.000 | 45.000 | 5 | 5.250 | 0.200 | 0.000 | 30.000 | 3.000 |
| 56 | 10 | 59.492 | 30.508 | 6.5 | 5.250 | 0.200 | 30.000 | 30.000 | 3.000 |
| 57 | 15 | 39.054 | 45.946 | 6.5 | 5.250 | 0.200 | 0.000 | 0.000 | 3.000 |
| 58 | 30 | 35.854 | 34.146 | 6.5 | 5.250 | 0.200 | 30.000 | 0.000 | 3.000 |
| 59 | 10 | 50.000 | 40.000 | 6.5 | 5.250 | 0.700 | 30.000 | 30.000 | 3.000 |
| 60 | 10 | 38.571 | 51.429 | 6.5 | 5.250 | 0.700 | 30.000 | 30.000 | 3.000 |
| 61 | 7.5 | 41.954 | 50.546 | 6.5 | 5.250 | 0.700 | 30.000 | 30.000 | 3.000 |
| 62 | 10 | 42.632 | 47.368 | 6.5 | 5.250 | 0.700 | 30.000 | 30.000 | 3.000 |
| 63 | 10 | 46.098 | 43.902 | 6.5 | 5.250 | 0.700 | 30.000 | 30.000 | 3.000 |
| 64 | 10 | 39.721 | 50.279 | 6.5 | 5.250 | 0.700 | 30.000 | 30.000 | 3.000 |
| 65 | 5 | 44.198 | 50.802 | 6.5 | 5.250 | 0.700 | 30.000 | 30.000 | 3.000 |
| 66 | 2.5 | 46.453 | 51.047 | 6.5 | 5.250 | 0.700 | 30.000 | 30.000 | 3.000 |
| 67 | 5 | 51.221 | 43.779 | 6.5 | 5.250 | 0.700 | 30.000 | 30.000 | 3.000 |
| 68 | 2.5 | 54.167 | 43.333 | 6.5 | 5.250 | 0.700 | 30.000 | 30.000 | 3.000 |
| 69 | 10 | 66.440 | 23.560 | 6.5 | 5.250 | 0.700 | 30.000 | 30.000 | 3.000 |
| 70 | 10 | 66.440 | 23.560 | 6.5 | 5.250 | 0.700 | 30.000 | 30.000 | 3.000 |
| 71 | 10 | 66.440 | 23.560 | 6.5 | 5.250 | 0.700 | 30.000 | 30.000 | 3.000 |
| 72 | 10 | 40.000 | 50.000 | 6.5 | 5.250 | 0.700 | 30.000 | 30.000 | 3.000 |
| 73 | 10 | 40.000 | 50.000 | 6.5 | 5.250 | 0.700 | 30.000 | 30.000 | 3.000 |
| 74 | 10 | 40.000 | 50.000 | 5.5 | 0.000 | 0.700 | 30.000 | 30.000 | 3.000 |
| 75 | 10 | 40.000 | 50.000 | 6.5 | 5.250 | 0.700 | 30.000 | 30.000 | 3.000 |
| 76 | 10 | 40.000 | 50.000 | 6.5 | 5.250 | 0.700 | 30.000 | 30.000 | 3.000 |
| 77 | 10 | 40.000 | 50.000 | 6.5 | 5.250 | 0.700 | 30.000 | 30.000 | 3.000 |
| 78 | 10 | 66.440 | 23.560 | 6.5 | 5.250 | 0.700 | 30.000 | 30.000 | 3.000 |
| 79 | 10 | 66.440 | 23.560 | 6.5 | 5.250 | 0.700 | 30.000 | 30.000 | 3.000 |
| 80 | 10 | 40.000 | 50.000 | 5.5 | 0.000 | 0.700 | 30.000 | 30.000 | 3.000 |
| 81 | 10 | 40.000 | 50.000 | 5.5 | 5.250 | 0.700 | 30.000 | 30.000 | 3.000 |
| 82 | 10 | 44.444 | 55.556 | 5.5 | 5.250 | 0.700 | 30.000 | 30.000 | 3.000 |
| 83 | 10 | 66.440 | 23.560 | 5.5 | 5.250 | 0.700 | 30.000 | 30.000 | 3.000 |
| 84 | 10 | 54.000 | 36.000 | 4 | 5.250 | 0.700 | 30.000 | 30.000 | 3.000 |
| 85 | 10 | 50.000 | 40.000 | 4 | 5.250 | 0.700 | 30.000 | 30.000 | 3.000 |
| 86 | 12.5 | 48.611 | 38.889 | 4 | 5.250 | 0.700 | 30.000 | 30.000 | 3.000 |
| 87 | 15 | 46.575 | 38.425 | 4 | 5.250 | 0.700 | 30.000 | 30.000 | 3.000 |
| 88 | 15 | 46.575 | 38.425 | 4 | 5.250 | 0.700 | 30.000 | 30.000 | 3.000 |
| 89 | 15 | 46.575 | 38.425 | 4 | 5.250 | 0.700 | 30.000 | 30.000 | 3.000 |
| 90 | 10 | 50.000 | 40.000 | 4.5 | 5.250 | 0.700 | 30.000 | 30.000 | 3.000 |
| 91 | 30 | 94.444 | 75.556 | 4 | 5.250 | 0.700 | 30.000 | 30.000 | 3.000 |
| 92 | 15 | 46.712 | 38.288 | 4 | 5.250 | 0.700 | 30.000 | 30.000 | 3.000 |
| 93 | 12 | 48.889 | 39.111 | 4 | 5.250 | 0.700 | 30.000 | 30.000 | 3.000 |
| 94 | 10 | 39.721 | 50.279 | 6.5 | 5.250 | 0.700 | 30.000 | 30.000 | 3.000 |
| 95 | 10 | 90.000 | 0.000 | 6.5 | 5.250 | 0.700 | 30.000 | 30.000 | 3.000 |
| 96 | 15 | 46.575 | 38.425 | 4 | 0.000 | 0.700 | 0.000 | 0.000 | 3.000 |
| 97 | 15 | 46.575 | 38.425 | 4 | 0.000 | 0.700 | 0.000 | 0.000 | 3.000 |
| 98 | 15 | 54.643 | 30.357 | 4 | 5.250 | 0.700 | 0.000 | 0.000 | 3.000 |
| 99 | 10 | 39.72 | 50.279 | 6.5 | 5.250 | 0.700 | 30.000 | 30.000 | 3.000 |
| 100 | 10 | 90.00 | | 6.5 | 5.250 | 0.700 | 30.000 | 30.000 | 3.000 |
| 101 | 15 | 46.57 | 38.425 | 4 | | 0.700 | | | 3.000 |
| 102 | 15 | 46.75 | 38.425 | 4 | | 0.700 | | | 3.000 |
| 103 | 15 | 54.64 | 30.357 | 4 | | 0.700 | | | 3.000 |

Table 16B: The table lists the specific components of the formulas listed above.

| Formula | Active | Lipid | Surfactant | Buffer | Antimicrobial | Antioxidants | Emollient | Chelator | Other |
|---|---|---|---|---|---|---|---|---|---|
| 1-4 | Antimicrobial | Sphingomyelin, e.g., | Tween 80 | Lactate | Benzyl alcohol | BHT (0.200) | Glycerol | EDTA | Ethanol |
| | | brain | | | | sodium | | | |
| | | | | | | metabisulfite | | | |
| | | | | | | (0.500) | | | |
| 5-7 | Antimicrobial | Sphingomyelin, | Brij 98 | Acetate | Benzyl alcohol | BHT (0.200) | Glycerol | EDTA | Ethanol |
| | | lauroyl | | | | sodium | | | |
| | | | | | | metabisulfite | | | |
| | | | | | | (0.500) | | | |
| 8-12 | Antimicrobial | Phosphatidyl choline + | Brij 98 | Phosphate | Benzyl alcohol | HTHQ | Glycerol | EDTA | Ethanol |
| | | Phosphatidylglycerol | | | | | | | |
| 13-16 | Antimicrobial | Phosphatidyl | Span 20 | Acetate | Benzyl alcohol | HTHQ | Glycerol | EDTA | Ethanol |
| | | choline + | | | | | | | |
| | | phosphatidylinositol | | | | | | | |
| 17-20 | Antimicrobial | Phosphatidyl | Tween 80 | Phosphate | Benzyl alcohol | BHT | Glycerol | EDTA | Ethanol |
| | | choline + | | | | | | | |
| | | phosphatidic acid | | | | | | | |
| 21-28 | Antimicrobial | Phosphatidyl | Cremophor | Lactate | Thimerosal | BHA | Glycerol | EDTA | Ethanol |
| | | choline | | | | | | | |
| 29-31 | Antimicrobial | Phosphatidyl | Tween 80 | Phosphate | Thimerosal | BHT (0.200) | Glycerol | EDTA | Ethanol |
| | | ethanolamine | | | | sodium | | | |
| | | | | | | metabisulfite | | | |
| | | | | | | (0.500) | | | |
| 32-37 | Antimicrobial | Phosphatidyl | Brij 98 | Acetate | Benzyl alcohol | BHT | Glycerol | EDTA | Ethanol |
| | | glycerol | | | | | | | |
| 38-40 | Antimicrobial | Phosphatidyl | Cremophor | phosphate | Benzyl alcohol | BHT (0.200) | Glycerol | EDTA | Ethanol |
| | | ethanolamine | | | | sodium | | | |
| | | | | | | metabisulfite | | | |
| | | | | | | (0.500) | | | |
| 41-47 | Antimicrobial | Phosphatidyl | Tween 80 | Propionate | Benzyl alcohol | BHT (0.200) | Glycerol | EDTA | Ethanol |
| | | glycerol | | | | sodium | | | |
| | | | | | | metabisulfite | | | |
| | | | | | | (0.500) | | | |
| 48-50 | Antimicrobial | Phosphatidyl serine | Brij 98 | Phosphate | Thimerosal | BHT (0.200) | Glycerol | EDTA | Ethanol |
| | | | | | | sodium | | | |
| | | | | | | metabisulfite | | | |
| | | | | | | (0.500) | | | |
| 51-58 | Antimicrobial | Phosphatidyl | Brij 98 | Acetate | Benzyl alcohol | BHT | Glycerol | EDTA | Ethanol |
| | | glycerol | | | | | | | |
| 59-68 | Antimicrobial | Phosphatidyl choline | Tween 80 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 69-71 | Antimicrobial | Phosphatidyl choline | Brij 98 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 72-73 | Antimicrobial | Phosphatidyl choline | Tween 80 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 74 | Antimicrobial | Phosphatidyl choline | Tween 80 | Acetate | | BHT (0.200) | Glycerol | EDTA | Ethanol |
| | | | | | | sodium | | | |
| | | | | | | metabisulfite | | | |
| | | | | | | (0.500) | | | |
| 75 | Antimicrobial | Phosphatidyl choline | Tween 80 | Phosphate | Paraben | BHT (0.200) | Glycerol | EDTA | Ethanol |
| | | | | | | sodium | | | |
| | | | | | | metabisulfite | | | |
| | | | | | | (0.500) | | | |
| 76 | Antimicrobial | Phosphatidyl choline | Brij 98 | Phosphate | Benzalkonium | BHT (0.200) | Glycerol | EDTA | Ethanol |
| | | | | | chloride | sodium | | | |
| | | | | | | metabisulfite | | | |
| | | | | | | (0.500) | | | |
| 77 | Antimicrobial | Phosphatidyl choline | Tween 80 | Phosphate | Paraben | BHT (0.200) | Glycerol | EDTA | Ethanol |
| | | | | | | sodium | | | |
| | | | | | | metabisulfite | | | |
| | | | | | | (0.500) | | | |
| 78 | Antimicrobial | Phosphatidyl choline | Brij 98 | Phosphate | Benzalkonium | BHT (0.200) | Glycerol | EDTA | Ethanol |
| | | | | | chloride | sodium | | | |
| | | | | | | metabisulfite | | | |
| | | | | | | (0.500) | | | |
| 79 | Antimicrobial | Phosphatidyl choline | Brij 98 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 80 | Antimicrobial | Phosphatidyl choline | Tween 80 | Acetate | | BHT (0.200) | Glycerol | EDTA | Ethanol |
| | | | | | | sodium | | | |
| | | | | | | metabisulfite | | | |
| | | | | | | (0.500) | | | |
| 81 | Antimicrobial | Phosphatidyl choline | Tween 80 | Acetate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 82-83 | Antimicrobial | Phosphatidyl choline | Tween 80 | Acetate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 84-88 | Antimicrobial | Phosphatidyl choline | Tween 80 | Acetate | Benzyl alcohol | BHA (0.200) | Glycerol | EDTA | Ethanol |
| | | | | | | sodium | | | |
| | | | | | | metabisulfite | | | |
| | | | | | | (0.500) | | | |
| 89 | Antimicrobial | Phosphatidyl choline | Tween 80 | Acetate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 90-93 | Antimicrobial | Phosphatidyl choline | Tween 80 | Acetate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 94 | Antimicrobial | Phosphatidyl choline | Tween 80 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 95 | Antimicrobial | Phosphatidyl choline | Tween 80 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 96-98 | Antimicrobial | Phosphatidyl choline | Tween 80 | Acetate | | BHT (0.200) | | EDTA | |
| | | | | | | sodium | | | |
| | | | | | | metabisulfite | | | |
| | | | | | | (0.500) | | | |
| 99 | Antimicrobial | Phosphatidyl choline | Tween 80 | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 100 | Antimicrobial | Phosphatidyl choline | | Phosphate | Benzyl alcohol | BHT (0.200) | Glycerol | EDTA | Ethanol |
| | | | | | | sodium | | | |
| | | | | | | metabisulfite | | | |
| | | | | | | (0.500) | | | |
| 101-103 | Antimicrobial | Phosphatidyl choline | Tween 80 | Phosphate | | BHT (0.200) | | EDTA | |
| | | | | | | sodium | | | |
| | | | | | | metabisulfite | | | |
| | | | | | | (0.500) | | | |

### Example Formulation 1

Formulation 1 comprises an antimicrobial (10 mg/g), sphingomyelin (brain) (47.944 mg/g) as a lipid, Tween 80 (42.056 mg/g) as a surfactant, lactate buffer (pH 4), benzyl alcohol (5.250 mg/g) as an antimicrobial agent, BHT (0.200 mg/g) and sodium metabisulfite (0.0500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 2

Formulation 2 comprises an antimicrobial (15 mg/g), sphingomyelin (brain) (53.750 mg/g) as a lipid, Tween 80 (31.250 mg/g) as a surfactant, lactate (pH 4) buffer, benzyl alcohol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (15.000 me/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 3

Formulation 3 comprises an antimicrobial (30 mg/g), sphingomyelin (brain) (90.561 mg/g) as a lipid, Tween 80 (79.439 mg/g) as a surfactant, lactate (pH 4) buffer, benzyl alcohol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 4

Formulation 4 comprises an antimicrobial (10 mg/g), sphingomyelin (brain) (47.944 mg/g) as a lipid, Tween 80 (42.056 mg/g) surfactant, lactate (pH 5) buffer, benzyl alcohol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 5

Formulation 5 comprises an antimicrobial (5 mg/g), sphingomyelin lauroyl (50.607 mg/g) as a lipid, Brij 98 (44.393 mg/g) as a surfactant, acetate (pH 5) butler, benzyl alcohol (5.250 mg/g) as an antimicrobial agent, BHT (0.200 mg/g) and sodium metabisulfite (0.500 mg/g) as antioxidants, EDTA (3.000 mg/g) as a chelating agent, and ethanol (10.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 6

Formulation 6 comprises an antimicrobial (30 mg/g), sphingomyelin lauroyl (90.561 mg/g) as a lipid, Brij 98 (79.439 mg/g) as a surfactant, acetate (pH 5) buffer, benzyl alcohol (5.250 mg/g) as antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 7

Formulation 7 comprises an antimicrobial (7.5 mg/g), sphingomyelin lauroyl (49.276 mg/g) as a lipid, Brij 98 (79.439 mg/g) as a surfactant, acetate (pH 6.5) buffer, benzyl alcohol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 8

Formulation 8 comprises an antimicrobial (15 mg/g), phosphatidyl choline and phosphatidyl glycerol (53.750 mg/g) a lipid, Brij 98 (31.250 mg/g) as a surfactant, phosphate (pH 6.5) buffer, benzyl alcohol (5.250 mg/g) as an antimicrobial agent, HTHQ (0.200 mg/g) as an antioxidant, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 9

Formulation 9 comprises an antimicrobial (30 mg/g), phosphatidyl choline and phosphatidyl glycerol (90.561 mg/g) as a lipid, Brij 98 (79.439 mg/g) as a surfactant, phosphate (pH 6.5) buffer, benzyl alcohol (5.250 mg/g) as an antimicrobial agent, HTHQ (0.200 mg/g) as an antioxidant, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 10

Formulation 10 comprises an antimicrobial (10 mg/g), phosphatidyl choline and phosphatidyl glycerol (41.351 mg/g) as a lipid, Brij 98 (48.649 mg/g) as a surfactant, phosphate (pH 4) buffer, benzyl alcohol (5.250 mg/g) as an antimicrobial agent, HTHQ (0.200 mg/g) as an antioxidant, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 11

Formulation 11 comprises an antimicrobial (15 mg/g), phosphatidyl choline and phosphatidyl glycerol (47.882 mg/g) as a lipid, Brij 98 (37.118 mg/g) as a surfactant, phosphate (pH 4) buffer, benzyl alcohol (5.250 mg/g) as an antimicrobial agent, HTHQ (0.200 mg/g) as an antioxidant, glycerol, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 12

Formulation 12 comprises an antimicrobial (30 mg/g), phosphatidyl choline and phosphatidyl glycerol (95.764 mg/g) as a lipid, Brij 98 (74.236 mg/g) as a surfactant, phosphate (pH 4) buffer, benzyl alcohol (5.250 mg/g) as an antimicrobial agent, HTHQ (0.200 mg/g) as an antioxidant, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 13

Formulation 13 comprises an antimicrobial (10 mg/g), phosphatidyl choline and phosphatidylinositol (66.676 mg/g) as a lipid, Span 20 (24.324 mg/g) as a surfactant, acetate (pH 5) buffer, benzyl alcohol (5.250 mg/g), HTHQ (0.200 mg/g) as an antioxidant, EDTA (3.000 mg/g) as a chelating agent, and ethanol (25.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 14

Formulation 14 comprises an antimicrobial (15 mg/g), phosphatidyl choline and phosphatidylinositol (62.027 mg/g) as a lipid, Span 20 (22.973 mg/g) as a surfactant, acetate (pH 5) buffer, benzyl alcohol (5.250 mg/g) as an antimicrobial agent, HTHQ (0.200 mg/g) as an antioxidant, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 15

Formulation 15 comprises an antimicrobial (30 mg/g), phosphatidyl choline and phosphatidylinositol (124.054 mg/g) as a lipid, Span 20 (45.946 mg/e) as a surfactant, acetate (pH 5) buffer, benzyl alcohol (5.250 mg/g) as an antimicrobial agent, HTHQ (0.200 mg/g) as an antioxidant, glycerol (30.000 mg/g), and EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 16

Formulation 16 comprises an antimicrobial (5 mg/g), phosphatidyl choline and phosphatidylinositol (62.687 mg/g) as a lipid, Span 20 (32.313 mg/g) as a surfactant, acetate (pH 6.5) buffer, benzyl alcohol (5.250 mg/g) as an antimicrobial agent, HTHQ (0.200 mg/g) as an antioxidant, glycerol (30.000 mg/g), EDTA (3.000 mg/g) as a chelating agent, wherein the antimicrobial is selected from the group consisting of itraconazole, ketoconazole, posaconazole, saperconazole. SCH-50002, terconazole, butenafine, and griseofulvin; and hydrates, solvates, and salts thereof.

### Example Formulation 17

Formulation 17 comprises an antimicrobial (15 mg/g), phosphatidyl choline and phosphatidic acid (41.853 mg/g) as a lipid. Tween 80 (43.147 mg/g) as a surfactant, phosphate (pH 6.5) buffer, benzyl alcohol (5.250 mg/g) as an antimicrobial agent, BHT (0.200 mg/g) as an antioxidant, glycerol (30.000 mg/g), EDTA (3.000 mg/g), and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 18

Formulation 18 comprises an antimicrobial (30 mg/g), phosphatidyl choline and phosphatidic acid (95.764 mg/g) as a lipid, Tween 80 (74.236 mg/g) as a surfactant, phosphate (pH 6.5) buffer, benzyl alcohol (5.250 mg/g) as an antimicrobial agent, BHT (0.200 mg/g) as an antioxidant, EDTA (3.000 mg/g), and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 19

Formulation 19 comprises an antimicrobial (15 mg/g), phosphatidyl choline and phosphatidic acid (47.882 mg/g) as a lipid, Tween 80 (37.118 mg/g) as a surfactant, phosphate (pH 6.5) buffer, benzyl alcohol (5.250 mg/g) as an antimicrobial agent, BHT (0.200 mg/g) as an antioxidant, and EDTA (3.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 20

Formulation 20 comprises an antimicrobial (10 mg/g), phosphatidyl choline and phosphatidic acid (45.000 mg/g) as a lipid, Tween 80 (45.000 mg/g) as a surfactant, phosphate (pH 6.5) buffer, benzyl alcohol (5.250 mg/g) as an antimicrobial agent, BHT (0.200 mg/g) as an antioxidant, and EDTA (3.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 21

Formulation 21 comprises an antimicrobial (10 mg/g), phosphatidyl choline (31.935 mg/g) as a lipid, cremophor (58.065 mg/g) as a surfactant, lactate (pH 5) buffer, thimerosal (5.250 mg/g) as an antimicrobial agent, BHA (0.200 mg/g) as an antioxidant, glycerol (30.000 mg/g), EDTA (3.000 mg/g) as a chelating agent, and ethanol (15.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 22

Formulation 22 comprises an antimicrobial (15 mg/g), phosphatidyl choline (42.500 mg/g) as a lipid, cremophor (42.500 mg/g) as a surfactant, lactate (pH 6.5) buffer, thimerosal (5.250 mg/g) as an antimicrobial agent, BHA (0.200 mg/g) as an antioxidant, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 23

Formulation 23 comprises an antimicrobial (10 mg/g), phosphatidyl choline (38.276 mg/g) as a lipid, cremophor (51.724 mg/g) as a surfactant, lactate (pH 4) buffer, thimerosal (5.250 mg/g) as an antimicrobial agent, BHA (0.200 mg/g) as an antioxidant, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 24

Formulation 24 comprises an antimicrobial (15 mg/g), phosphatidyl choline (42.500 mg/g) as a lipid, cremophor (42.500 mg/g) as a surfactant, lactate (pH 4) buffer, thimerosal (5.250 mg/g) as an antimicrobial agent, BHA (0.200 mg/g) as an antioxidant, EDTA (3.000 mg/g) as a chelating agent, and ethanol (15.000 mg/g), wherein the antimicrobial is selected from the group consisting of itraconazole, ketoconazole, posaconazole, saperconazule, SCH-50002, terconazole, butenafine, and griseofulvin; and hydrates, solvates, and salts thereof.

### Example Formulation 25

Formulation 25 comprises an antimicrobial (30 mg/g), phosphatidyl choline (85.000 mg/g) as a lipid, cremophor (85.000 mg/g) as a surfactant, lactate (pH 4) buffer, thimerosal (5.250 mg/g) as an antimicrobial agent, BHA (0.200 mg/g) as an antioxidant, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 26

Formulation 26 comprises an antimicrobial (10 mg/g), phosphatidyl choline (38.276 mg/g) as a lipid, cremophor (51.276 mg/g) as a surfactant, lactate (pH 5) buffer, thimerosal (5.250 mg/g) as an antimicrobial agent, BHA (0.200 mg/g) as an antioxidant, and EDTA (3.000 mg/g) as a chelating agent, wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 27

Formulation 27 comprises an antimicrobial (15 mg/g), phosphatidyl choline (36.429 mg/g) as a lipid, cremophor (48.571 mg/g) as a surfactant, lactate (pH 5) buffer, thimerosal (5.250 mg/g) as an antimicrobial agent, BHA (0.200 mg/g) as an antioxidant. EDTA (3.000 mg/g) as a chelating agent, and ethanol (30.000 mg/g); wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 28

Formulation 28 comprises an antimicrobial (30 mg/g), phosphatidyl choline (72.299 mg/g) as a lipid, cremophor (97.701 mg/g) as a surfactant, lactate (pH 5) buffer, thimerosal (5.250 mg/g) as an antimicrobial agent, BHA (0.200 mg/g) as an antioxidant, EDTA (3.000 mg/g) as a chelating agent, and ethanol (15.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 29

Formulation 29 comprises an antimicrobial (7.5 mg/g), phosphatidyl ethanolamine (46.250 mg/g) as a lipid. Tween 80 (46.250 mg/g) as a surfactant, phosphate (pH 6.5) buffer, thimerosal (5.250 mg/g) as an antimicrobial agent, BHT (0.200 mg/g) and sodium metabisulfite (0.500 mg/g) as an antioxidant, EDTA (3.000 mg/g) as a chelating agent, and ethanol (20.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 30

Formulation 30 comprises an antimicrobial (15 mg/g), phosphatidyl ethanolamine (38.804 mg/g) as a lipid, Tween 80 (46.196 mg/g) as a surfactant, phosphate (pH 6.5) buffer, thimerosal (5.250 mg/g) as an antimicrobial agent, BHT (0.200 mg/g) and sodium metabisulfite (0.500 mg/g) as an antioxidant, glycerol (15.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 31

Formulation 31 comprises an antimicrobial (30 mg/g), phosphatidyl ethanolamine (36.667 mg/g) as a lipid, Tween 80 (33.333 mg/g) as a surfactant, phosphate (pH 6.5) buffer, thimerosal (5.250 mg/g) as an antimicrobial agent, BHT (0.200 mg/g) and sodium metabisulfite (0.500 mg/g) as antioxidants, glycerol (30.000 mg/g). EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 32

Formulation 32 comprises an antimicrobial 10 mg/g, phosphatidyl glycerol (23.333 mg/g) as a lipid, Brij 98 (66.667 mg/g) as a surfactant, acetate (pH 4) buffer, benzyl alcohol (5.250 mg/g) as an antimicrobial agent, BHT (0.200 mg/g) as an antioxidant, and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 33

Formulation 33 comprises an antimicrobial (12.5 mg/g), phosphatidyl glycerol (45.833 mg/g) as a lipid, Brij 98 (41.667 mg/g) as a surfactant, acetate (pH 4) butler, benzyl alcohol (5.250 mg/g) as an antimicrobial agent, BHT (0.200 mg/g) as an antioxidant, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 34

Formulation 34 comprises an antimicrobial (30 mg/g), phosphatidyl glycerol (31.957 mg/g) as a lipid, Brij 98 (38.043 mg/g) as a surfactant, acetate (pH 4) buffer, benzyl alcohol (5.250 mg/g) as an antimicrobial agent, BHT (0.200 mg/g) as an antioxidant, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 35

Formulation 35 comprises an antimicrobial (10 mg/g), phosphatidyl glycerol (47.143 mg/g) as a lipid, Brij 98 (42.857 mg/g) as a surfactant, acetate (pH 5) buffer, benzyl alcohol (5.250 mg/g) as an antimicrobial agent, BHT (0.200 mg/g) as an antioxidant, glycerol (30.000 mg/g), EDTA (3.000 mg/g) as a chelating agent, and ethanol (25.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 36

Formulation 36 comprises an antimicrobial (15 mg/g), phosphatidyl glycerol (96.905 mg/g) as a lipid, Brij 98 (88.095 mg/g) as a surfactant, acetate (pH 5) buffer, benzyl alcohol (5.250 mg/g) as an antimicrobial agent, BHT (0.200 mg/g) as an antioxidant, glycerol (30.000 mg/g), EDTA (3.000 mg/g) as a chelating agent, and ethanol (20.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 37

Formulation 37 comprises an antimicrobial (30 mg/g), phosphatidyl glycerol (31.957 mg/g) as a lipid, Brij 98 (38.043) as a surfactant, acetate (pH 5) buffer, benzyl alcohol (5.250 mg/g) as an antimicrobial agent, BHT (0.200 mg/g) as an antioxidant, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 38

Formulation 38 comprises an antimicrobial (10 mg/g), phosphatidyl ethanolamine (35.455 mg/g) as a lipid, cremophor (54.545 mg/g) as a surfactant, phosphate (pH 6.5) buffer, benzyl alcohol (5.250 mg/g) as an antimicrobial agent, BHT (0.200 mg/g) and sodium metabisulfite (0.500 mg/g) as antioxidants, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 39

Formulation 39 comprises an antimicrobial (15 mg/g), phosphatidyl ethanolamine (84.457 mg/g) as a lipid, cremophor (100.543 mg/g) as a surfactant, phosphate (pH 6.5) butler, benzyl alcohol (5.250 mg/g) as an antimicrobial agent, BHT (0.200 mg/g) and sodium metabisulfite (0.500 mg/g) as antioxidants, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof,

### Example Formulation 40

Formulation 40 comprises an antimicrobial (30 mg/g), phosphatidyl ethanolamine (89.048 mg/g) as a lipid, cremophor (80.952 mg/g) as a surfactant, phosphate (pH 6.5) buffer, benzyl alcohol (5.250 mg/g), BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 41

Formulation 41 comprises an antimicrobial (10 mg/g), phosphatidyl glycerol (41.087 mg/g) as a lipid. Tween 80 (48.913 mg/g) as a surfactant, propionate (pH 4) buffer, benzyl alcohol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 42

Formulation 42 comprises an antimicrobial (15 mg/g), phosphatidyl glycerol (45.280 mg/g) as a lipid, Tween 80 (39.720 mg/g) as a surfactant, propionate (pH 4) buffer, benzyl alcohol (5.250 mg/g) as an antimicrobial agent, BHT (0.200 mg/g) and sodium metabisulfite (0.500 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 43

Formulation 43 comprises an antimicrobial (30 mg/g), phosphatidyl glycerol (107.500 mg/g) as a lipid, Tween 80 (62.500 mg/g) as a surfactant, propionate (pH 4) buffer, benzyl alcohol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 44

Formulation 44 comprises an antimicrobial (5 mg/g), phosphatidyl glycerol (77.243 mg/g) as a lipid, Tween 80 (67.757 mg/g) as a surfactant, propionate (pH 4) buffer, benzyl alcohol (5.250 mg/g) as an antimicrobial agent, BHT (0.200 mg/g) and sodium metabisulfite (0.500 mg/g) as antioxidants, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 45

Formulation 45 comprises an antimicrobial (15 mg/g), phosphatidyl glycerol (45.280 mg/g) as a lipid, Tween 80 (39.720 mg/g) as a surfactant, propionate (pH 5) buffer, benzyl alcohol (5.250 mg/g) as an antimicrobial agent, BHT (0.200 mg/g) and sodium metabisulfite (0.500 mg/g) as antioxidants, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 46

Formulation 46 comprises an antimicrobial (30 mg/g), phosphatidyl glycerol (90.561 mg/g) as a lipid, Tween 80 (79.439 mg/g) as a surfactant, propionate (pH 5) buffer, benzyl alcohol (5.250 mg/g) as an antimicrobial agent, BHT (0.200 mg/g) and sodium metabisulfite (0.500 mg/g) as antioxidants, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 47

Formulation 47 comprises an antimicrobial (10 mg/g), phosphatidyl glycerol (47.944 mg/g) as a lipid, Tween 80 (42.056 mg/g) as a surfactant, propionate (pH 5) buffer, benzyl alcohol (5.250 mg/g) as an antimicrobial agent, BHT (0.200 mg/g) and sodium metabisulfite (0.500 mg/g) as antioxidants, EDTA (3.000 mg/g) as a chelating agent, and ethanol (10.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 48

Formulation 48 comprises an antimicrobial (5 mg/g), phosphatidyl serine (50.607 mg/g) as a lipid, Brij 98 (44.393 mg/g) as a surfactant, phosphate (pH 5.5) buffer, thimerasol (5.250 mg/g) as an antimicrobial agent, BHT (0.200 mg/g) and sodium metabisulfite (0.500 mg/g) as antioxidants, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 49

Formulation 49 comprises an antimicrobial (30 mg/g), phosphatidyl serine (107.500 mg/g) as a lipid, Brij 98 (62.500 mg/g) as a surfactant, phosphate (pH 5.5) buffer, thimerasol (5.250 mg/g) as an antimicrobial agent, BHT (0.200 mg/g) and sodium metabisulfite (0.500 mg/g) as antioxidants, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 50

Formulation 50 comprises an antimicrobial (10 mg/g), phosphatidyl serine (47.944 mg/g) as a lipid, Brij 98 (42.056 mg/g) as a surfactant, phosphate (pH 5.5) buffer, thimerasol (5.250 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 51

Formulation 51 comprises an antimicrobial (15 mg/g), phosphatidyl glycerol (46.364 mg/g) as a lipid, Brij 98 (38.636 mg/g) as a surfactant, acetate (pH 4) buffer, benzyl alcohol (5.250 mg/g) as an antimicrobial, BHT (0.200 mg/g) as an antioxidant, glycerol (30.000 mg/g), EDTA (3.000 mg/g) as a chelating agent, and ethanol (25.000 mg/g), wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 52

Formulation 52 comprises an antimicrobial (15 mg/g), phosphatidyl glycerol (46.364 mg/g) as a lipid, Brij 98 (38.636 mg/g) as a surfactant, acetate (pH 4) buffer, benzyl alcohol (5.250 mg/g) as an antimicrobial, BHT (0.200 mg/g) as an antioxidant, EDTA (3.000 mg/g) as a chelating agent, and ethanol (20.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 53

Formulation 53 comprises an antimicrobial (10 mg/g), phosphatidyl glycerol (46.098 mg/g) as a lipid, Brij 98 (43.902 mg/g) as a surfactant, acetate (pH 5) buffer, benzyl alcohol (5.250 mg/g) as an antimicrobial, BHT (0.200 mg/g) as an antioxidant, glycerol (15.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 54

Formulation 54 comprises an antimicrobial (15 mg/g), phosphatidyl glycerol (43.537 mg/g) as a lipid, Brij 98 (41.463 mg/g) as a surfactant, acetate (pH 5) buffer, benzyl alcohol (5.250 mg/g) as an antimicrobial, BHT (0.200 mg/g) as an antioxidant, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 55

Formulation 55 comprises an antimicrobial (10 mg/g), phosphatidyl glycerol (45.000 mg/g) as a lipid, Brij 98 (45.000 mg/g) as a surfactant, acetate (pH 5) buffer, benzyl alcohol (5.250 mg/g) as an antimicrobial, BHT (0.200 mg/g) as an antioxidant, EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 56

Formulation 56 comprises an antimicrobial (10 mg/g), phosphatidyl glycerol (59.492 mg/g) as a lipid, Brij 98 (30.508 mg/g) as a surfactant, acetate (pH 6.5) buffer benzyl alcohol (5.250 mg/g) as an antimicrobial, BHT (0.200 mg/g) as an antioxidant, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 57

Formulation 57 comprises an antimicrobial (15 mg/g), phosphatidyl glycerol (39.054 mg/g) as a lipid, Brij 98 (45.946 mg/g) as a surfactant, acetate (pH 6.5) buffer, benzyl alcohol (5.250 mg/g) as an antimicrobial, BI IT (0.200 mg/g) as an antioxidant, and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 58

Formulation 58 comprises an antimicrobial (30 mg/g), phosphatidyl glycerol (35.854 mg/g) as a lipid, Brij 98 (34.146 mg/g) as a surfactant, acetate (pH 6.5) buffer, benzyl alcohol (5.250 mg/g) as an antimicrobial, BHT (0.200 mg/g) as an antioxidant, glycerol (30.000 mg/g), and EDTA (3.000 mg/g) as a chelating agent, wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 59

Formulation 59 comprises an antimicrobial (10 mg/g), phosphatidyl choline (50.000 mg/g) as a lipid, Tween 80 (40.000 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 60

Formulation 60 comprises an antimicrobial (10 mg/g), phosphatidyl choline (38.571 mg/g) as a lipid, Tween 80 (51.429 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g), and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 61

Formulation 61 comprises an antimicrobial (7.5 mg/g), phosphatidyl choline (41.954 mg/g) as phospholipid, Tween 80 (50.546 mg/g) as surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g), and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 62

Formulation 62 comprises an antimicrobial (10 mg/g), phosphatidyl choline (42.632 mg/g) as a lipid, Tween 80 (47.368 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 63

Formulation 63 comprises an antimicrobial (10 mg/g phosphatidyl choline (46.098 mg/0 as a lipid. Tween 80 (43.902 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 64

Formulation 64 comprises an antimicrobial (10 mg/g), phosphatidyl choline (39.721 mg/g) as a lipid, Tween 80 (50.279 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 65

Formulation 65 comprises an antimicrobial (5 mg/g), phosphatidyl choline (44.198 mg/g) as a lipid, Tween 80 (50.802 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 66

Formulation 66 comprises an antimicrobial (2.5 mg/g), phosphatidyl choline (46.453 mg/g) as a lipid, Tween 80 (51.047 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griscofulvin, and hydrates, solvates, and salts thereof.

### Example Formulation 67

Formulation 67 comprises an antimicrobial (5 mg/g), phosphatidyl choline (51.221 mg/g) as a lipid, Tween 80 (43.779 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 68

Formulation 68 comprises an antimicrobial (2.5 mg/g), phosphatidyl choline (54.167 mg/g) as a lipid. Tween 80 (43.333 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 69

Formulation 69 comprises an antimicrobial (10 mg/g), phosphatidyl choline (66.440 mg/g) as a lipid, Brij 98 (23.560 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 69 is an emulsion.

### Example Formulation 70

Formulation 70 comprises an antimicrobial (10 mg/g), phosphatidyl choline (66.440 mg/g) as a lipid, Brij 98 (23.560 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 70 is a suspension.

### Example Formulation 71

Formulation 71 comprises an antimicrobial (10 mg/g), phosphatidyl choline (66.440 mg/g) as a lipid, Brij 98 (23.560 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 72

Formulation 72 comprises an antimicrobial (10 mg/g), phosphatidyl choline (40.000 mg/g) as a lipid, Tween 80 (50.000 mg/g) as a surfactant, phosphate (pH 6.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 72 is an emulsion.

### Example Formulation 73

Formulation 73 comprises an antimicrobial (10 mg/g), phosphatidyl choline (40.000 mg/g) as a lipid, Tween 80 (50.000 mg/g) as a surfactant, phosphate (pH 6.5) butler, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 73 is a suspension.

### Example Formulation 74

Formulation 74 comprises an antimicrobial (10 mg/g phosphatidyl choline (40.000 mg/g) as a lipid, Tween 80 (50.000 mg/g) as a surfactant, acetate (pH 5.5) hurler, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 75

Formulation 75 comprises an antimicrobial (10 mg/g), phosphatidyl choline (40.000 mg/g) as a lipid, Tween 80 (50.000 mg/g) as a surfactant, phosphate (pH 6.5) buffer, paraben (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 76

Formulation 76 comprises an antimicrobial (10 mg/g), phosphatidyl choline (40.000 mg/g) as a lipid, Brij 98 (50.000 mg/g) as a surfactant, phosphate (pH 6.5) buffer, benzalkonium chloride (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 77

Formulation 77 comprises an antimicrobial (10 mg/g), phosphatidyl choline (40.000 mg/g) as a lipid, Tween 80 (50.000 mg/g) as a surfactant, phosphate (pH 6.5) buffer, paraben (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 78

Formulation 78 comprises an antimicrobial (10 mg/g), phosphatidyl choline (66.440 mg/g) as a lipid, Brij 98 (23.560 mg/g) as a surfactant, phosphate (pH 6.5) buffer, benzalkonium chloride (5.250 mg/g) as an antimicrobial, BHT (0.200 mg/g) and sodium metabisulfite (0.500 mg/g) as antioxidants, glycerol (30.000 mg/g), EDTA (3.00 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 79

Formulation 79 comprises an antimicrobial (10 mg/g), phosphatidyl choline (66.440 mg/g) as a lipid, Brij 98 (23.560 mg/g) as a surfactant, phosphate (pH 6.5) buffer, benzyl alcohol (5.250 mg/g) as an antimicrobial. BI IT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 80

Formulation 80 comprises an antimicrobial (10 mg/g), phosphatidyl choline (40.000 mg/g) as a lipid, Tween 80 (50.000 mg/g) as a surfactant, acetate (pH 5.5) buffer, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 81

Formulation 81 comprises an antimicrobial (10 mg/g), phosphatidyl choline (40.000 mg/g) as a lipid, Tween 80 (50.000 mg/g) as a surfactant, acetate (pH 5.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin, and hydrates, solvates, and salts thereof.

### Example Formulation 82

Formulation 82 comprises an antimicrobial (10 mg/g), phosphatidyl choline (44.444 mg/g) as a lipid, Tween 80 (55.556 mg/g) as a surfactant, acetate (pH 5.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 83

Formulation 83 comprises an antimicrobial (10 mg/g), phosphatidyl choline (66.440 mg/g) as a lipid, Tween 80 (23.560 mg/g) as a surfactant, acetate (pH 5.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 84

Formulation 84 comprises an antimicrobial (10 mg/g), phosphatidyl choline (54.000 mg/g) as a lipid, Tween 80 (36.000 mg/g) as a surfactant, acetate (pH 4) buffer, benzyl alcohol (5.250 mg/g) as an antimicrobial, BHA (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 85

Formulation 85 comprises an antimicrobial (10 mg/g), phosphatidyl choline (50.000 mg/g) as a lipid, Tween 80 (40.000 mg/g) as a surfactant, acetate (pH 4) buffer, benzyl alcohol (5.250 mg/g) as an antimicrobial, BHA (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 86

Formulation 86 comprises an antimicrobial (12.5 mg/g), phosphatidyl choline (48.611 mg/g) as a lipid, Tween 80 (38.889 mg/g) as a surfactant, acetate (pH 4) buffer, benzyl alcohol (5.250 mg/g) as an antimicrobial, BHA (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 87

Formulation 87 comprises an antimicrobial (15 mg/g), phosphatidyl choline (46.575 mg/g) as a lipid, Tween 80 (38.425 mg/g) as a surfactant, acetate (pH 4) buffer, benzyl alcohol (5.250 mg/g) as an antimicrobial, BHA (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 87 is an emulsion.

### Example Formulation 88

Formulation 88 comprises an antimicrobial (15 mg/g), phosphatidyl choline (46.575 mg/g) as a lipid, Tween 80 (38.425 mg/g) as a surfactant, acetate (pH 4) buffer, benzyl alcohol (5.250 mg/g) as an antimicrobial, BHA (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 88 is suspension.

### Example Formulation 89

Formulation 89 comprises an antimicrobial (15 mg/g), phosphatidyl choline (46.575 mg/g) as a lipid, Tween 80 (38.425 mg/g) as a surfactant, acetate (pH 4) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 90

Formulation 90 comprises an antimicrobial (10 mg/g), phosphatidyl choline (50.000 mg/0 as a lipid, Tween 80 (40.000 mg/g) as a surfactant, acetate (pH 4.5) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 91

Formulation 91 comprises an antimicrobial (30 mg/g), phosphatidyl choline (94.444 mg/g) as a lipid, Tween 80 (75.556 mg/g) as a surfactant, acetate (pH 4) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 92

Formulation 92 comprises an antimicrobial (15 mg/g), phosphatidyl choline (46.712 mg/g) as a lipid, Tween 80 (38.288 mg/g) as a surfactant, acetate (pH 4) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 93

Formulation 93 comprises an antimicrobial (12 mg/g), phosphatidyl choline (48.889 mg/g) as a lipid, Tween 80 (39.111 mg/g) as a surfactant, acetate (pH 4) buffer, benzyl alcohol (5.250 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 94

Formulation 94 comprises an antimicrobial (10 mg/g), phosphatidyl choline (39.721 mg/g) as a lipid, Tween 80 (50.279 mg/g) as a surfactant, phosphate (pH 6.5) buffer, benzyl alcohol (5.25 mg/g) as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griscofulvin; and hydrates, solvates, and salts thereof.

### Example Formulation 95

Formulation 95 comprises an antimicrobial (10 mg/g), phosphatidyl choline (90.000 mg/g) as a lipid, phosphate buffer (pH 6.5), benzyl alcohol as an antimicrobial, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 96

Formulation 96 comprises an antimicrobial (15 mg/g), phosphatidyl choline (46.575 mg/g) as a lipid, Tween 80 (38.425 mg/g) as a surfactant, phosphate (pH 4) buffer. BHT (0.500 mg/g) and sodium metabisulfite (0.200 mg/g) as antioxidants, and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 96 is an emulsion.

### Example Formulation 97

Formulation 97 comprises an antimicrobial (15 mg/g), phosphatidyl choline (46.575 mg/g) as a lipid, Tween 80 (38.425 mg/g) as a surfactant, phosphate (pH 4) buffer, BHT (0.500 mg/g) and sodium metabisulfite (0.200 mg/g) as antioxidants, and EDTA (3.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof. Example formulation 97 is a suspension.

### Example Formulation 98

Formulation 98 comprises an antimicrobial (15 mg/g), phosphatidyl choline (54.643 mg/g) as a lipid, Tween 80 (30.357 mg/g) as a surfactant, phosphate (pH 4) buffer, BHA (0.500 mg/g) and sodium metabisulfite (0.200 mg/g) as antioxidants, and EDTA (3.000 mg/g) as a chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 99

Formulation 99 comprises an antimicrobial (10 mg/g), phosphatidyl choline (39.72 mg/g) as a lipid, Tween 80 (50.279 mg/g) as surfactant, phosphate (pH 6.5) buffer, benzyl alcohol (5.25 mg/g) as an antimicrobial, BHT (0.200 mg/g) and sodium metabisulfite (0.500 mg/g) as antioxidants, glycerol (30.000 mg/g) as emollient, EDTA (3.000 mg/g) as the chelating agent, and ethanol (30.000 mg/g), wherein the antimicrobial is selected from the group consisting of itraconazole, ketoconazole, posaconazole, saperconazole. SCH-50002, terconazole, butenafine, and griseofulvin; and hydrates, solvates, and salts thereof.

### Example Formulation 100

Formulation 100 comprises an antimicrobial (10 mg/g), phosphatidyl choline (90.00 mg/g) as a lipid, phosphate (pH 6.5) buffer, benzyl alcohol as antimicrobial, BHT (0.200 mg/g) and sodium metabisulfite (0.500 mg/g) as antioxidants, glycerol (30.000 mg/g) as emollient, EDTA (3.000 mg/g) as the chelating agent, and ethanol (30.000 mg/g), wherein the antimicrobial is selected from the group consisting of itraconazole, ketoconazole, posaconazole, saperconazole, SCH-50002, terconazole, butenafine, and griseofulvin; and hydrates, solvates, and salts thereof.

### Example Formulation 101

Formulation 101 comprises an antimicrobial 15 mg/g), phosphatidyl choline (46.57 mg/g) as a lipid, Tween 80 (38.425 mg/g) as a surfactant, phosphate (pH 4) buffer, BHT (0.500 mg/g) and sodium metabisulfite (0.200 mg/g) as antioxidants, and EDTA (3.000 mg/g) as the chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof. Formulation 101 is formulated as an emulsion.

### Example Formulation 102

Formulation 102 comprises an antimicrobial (15 mg/g), phosphatidyl choline (46.57 mg/g) as a lipid, Tween 80 (38.425 mg/g) as a surfactant, phosphate (pH 4) buffer, BHT (0.500 mg/g) and sodium metabisulfite (0.200 mg/g) as antioxidants, and EDTA (3.000 mg/g) as the chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof. Formulation 102 as a suspension.

### Example Formulation 103

Formulation 103 comprises an antimicrobial (15 mg/g), phosphatidyl choline (54.64 mg/g) as a lipid, Tween 80 (30.357 mg/g) as a surfactant, phosphate (pH 4) buffer, BHA (0.500 mg/g) and sodium metabisulfite (0.200 mg/g) as antioxidants, EDTA (3.000 mg/g) as the chelating agent, wherein the antimicrobial is selected from the group consisting of itraconazole, ketoconazole, posaconazole, saperconazole, SCH-50002, terconazole, butenafine, and griseofulvin; and hydrates, solvates, and salts thereof.

Example Formulations 1 through 103 may also optionally include thickeners such as pectin, xanthan gum, HPMC gel, methylcellulose or carbopol. Example Formulations 1 through 103 may contain an antimicrobial provided herein, including single enantiomers, mixtures of enantiomers, and mixtures of diastereomers thereof; and pharmaceutically acceptable solvates, hydrates, and salts thereof.

What is claimed is:

1. A method for reducing the proliferation or viability of a mycotic agent comprising contacting said mycotic agent with an effective amount of an antifungal agent, wherein said antifungal agent is formulated with a phospholipid and a surfactant, wherein said antifungal agent is selected from those listed in Table 1, and wherein said antifungal agent is adsorbed by the phospholipid membranes of the Spitzenkorper or Polarisome regions of the hypha of said mycotic agent.

2. The method of claim 1 wherein said mycotic agent is selected from the group consisting of Trichophyton rubrum, Trichophyton mentagrophytes, Epidermophytonfjloccusum, Candida albicans, Dermatophytes, Malassezia furfur, Microsporum canis, Trichophyton tonsurans, Microsporum audouini, Microsporum gypseum, Trichophyton rubrum, Trichophyton tonsurans, Trichophyton mentagrophytes, Trichophyton interdigitalis, Trichophyton verrucosum, Trichophyton sulphureum, Trichophyton schoenleini, Trichophyton megnini, Trichophyton gallinae, Trichophyton crateriform, Trichomonas and Haemophilus vaginalis, Aspergillus fumigatus, Aspergillus flavus, Aspergillus clavatus, Trypanosoma brucei, and Trypanosoma cruzi.

3. The method of claim 1, wherein said antifungal agent is terbinafine, flucanazole, or voriconazole.

4. The method of claim 1, wherein said antifungal agent is administered to an animal in order to reduce the proliferation or viability of a mycotic agent that has infected said animal.

5. The method of claim 1, wherein said antifungal agent is delivered to a plant in order to reduce the proliferation or viability of a mycotic agent that has infected said plant.

6. The method of claim 1, wherein the molar ratio of phospholipid to surfactant (mol lipid/mol surfactant) is from about 1:2 to about 10:1.

7. The method of claim 1, wherein the formulation contains from about 1.0% to about 30.0% by weight phospholipid, and from about 1.0% to about 50.0% by weight surfactant.

8. The method of claim 1, wherein the phospholipid is phosphatidylcholine, and the surfactant is a polyoxyethylene-sorbitan-fatty acyl ester, a polyoxyethylene-sorbitan-fatty ether, a polyhydroxyethylene-fatty monoacyl ester, a polyhydroxyethylene-fatty diacyl ester, or a polyhydroxyethylene-fatty ether.

9. The method of claim 8, wherein the surfactant is polysorbate 80 (Tween 80), polysorbate 60 (Tween 60), polysorbate 40 (Tween 40), polysorbate 20 (Tween 20), Brij 98, Brij 35, Simulsol-2599, Myrj-52, TritonX100, or Cremophor.

10. A method of screening compounds for antimicrobial activity comprising contacting a microbial agent with an effective amount of a compound, wherein said compound is formulated with a phospholipid and a surfactant, and detecting a reduction in the proliferation or viability of said microbial agent, wherein said compound is adsorbed by the phospholipid membranes of the Spitzenkorper or Polarisome regions of the hypha of said microbial agent.

11. The method of claim 10, wherein said microbial agent is a fungus, a bacterium, or a mycoplasma.

12. A method for reducing the proliferation or viability of a bacterium, comprising contacting said bacterium with an effective amount of an antibacterial agent, wherein said antibacterial agent is formulated with a phospholipid and a surfactant, and wherein said antibacterial agent is adsorbed by the phospholipid membranes of the bacterium.

13. The method of claim 12, wherein said antibacterial agent is selected from the group consisting of benzyl alcohol, methyl paraben ethanol, isopropanol, glutaraldehyde, formaldehyde, chlorine compounds, iodine compounds, hydrogen peroxide, peracetic acid, ethylene oxide, triclocarban, chlorhexidine, alexidine, triclosan, hexachlorophene, polymeric biguanides, formaldehyde, aminoglycoside antibiotics, glycopeptides, amphenicol antibiotics, ansamycin antibiotics, cephalosporins, cephamycins oxazolidinones, penicillins, quinolones, streptogamins, tetracycline, and analogs thereof.

14. The method of claim 12, wherein said bacterium is selected from the group consisting of E. coli, Klebsiella, Staphylococcus, Streptococcus, Haemophilus influenzae, Neisseria gonorrhoeae, Pseudomonas, Clostridium, Enterococcus, Bacillus, Acinetobacter baumannii, M tuberculosis, Chlamydia, N gonorrhea, Shigella, Salmonella, Proteus, Gardnerella, Nocardia, Nocardia asteroides, Planococcus, Corynebacteria, Rhodococcus, Vibrio, Cholera, Treponema pallidua, Pseudomonas, Bordetella pertussis, Brucella, Franciscella tulorensis, Helicobacter pylori, Leptospria interrogaus, Legionella pneumophila, Yersinia, Pneumococcus, Meningococcus, Hemophilus irifluenza, Toxoplasma gondic, Complylobacteriosis, Moraxella catarrhalis, Donovanosis, and Actinomycosis.

15. The method of claim 14, wherein said mycobacterium is Mycobacterium tuberculosis, and said antibacterial agent is selected from the group consisting of isoniazid, rifampin, pyrazinamide, ethambutol, and streptomycin.

16. The method of claim 12, wherein said bacterium is a mycoplasma selected from the group consisting of M. buccale, M. faucium, M. fermentans, M. Genitalium, M. hominis, M. lipophilum, M. oral, M. penetrans, M. pneumoniae, M. salivarium, and M. spermatophilum, and said antibacterial agent is selected from the group consisting of erythromycin, azithromycin, clarithromycin, tetracycline, doxycycline, minocycline, clindamycin, ofloxacin, and chloramphenicol.

17. The method of claim 12, wherein said antibacterial agent is administered to an animal in order to reduce the proliferation or viability of a bacterium that has infected said animal.

18. The method of claim 12, wherein said antibacterial agent is delivered to a plant in order to reduce the proliferation or viability of a bacterium that has infected said plant.

19. A method of preventing the development of inhalation anthrax and/or treating inhalation anthrax in a human subject that has been exposed to Bacillus anthracis spores, said method comprising administering to said human subject a composition comprising an antibacterial agent that is formulated with a phospholipid and a surfactant, and wherein said antibacterial agent is adsorbed by the phospholipid membrane of said Bacillus anthracis.

20. A method of treating pneumonia in a human subject that has been infected with Mycoplasma pneumoniae, and/or tuberculosis in a human subject that has been infected with Mycobacterium tuberculosis, said method comprising administering to said human subject a composition comprising an antibacterial agent that is formulated with a phospholipid and a surfactant, and wherein said antibacterial agent is adsorbed by the phospholipid membrane of said Mycoplasma pneumoniae and/or the phospholipid membrane of said Mycobacterium tuberculosis, respectively.
